(19) United States
(12) Patent Application Publication
(10) Pub. No.: US 2023/0133281 A1
(43) Pub. Date: May 4, 2023

(54) MATERIALS AND METHODS INCLUDING FOR SEX SELECTION

(71) Applicant: CHROMOXYION PTY LTD, Karana Downs, Queensland (AU)

(72) Inventor: Sharon Catherine DE WET, Karana Downs, Queensland (AU)

(21) Appl. No.: 16/768,577

(22) PCT Filed: Dec. 3, 2018
(86) PCT No.: PCT/AU2018/000243

§ 371 Date: May 29, 2020

Related U.S. Application Data
(60) Provisional application No. 62/594,124, filed on Dec. 4, 2017.
(60) Provisional application No. 62/594,153, filed on Dec. 4, 2017.

(51) Int. Cl. A01K 67/02; C07K 16/28

(52) U.S. Cl. CPC A01K 67/02; C07K 16/28; A01K 2227/10; C07K 2317/565; C07K 2317/33

(57) ABSTRACT

A method comprising the steps of: 1. optionally subjecting spermatozoa to a treatment step; 2. subjecting the spermatozoa of step 1 to a sex selection step so as to select for either female or male spermatozoa of interest; 3. carrying out a fertilisation step using the spermatazoa of interest of step 2 to produce at least one oocyte, blastocyst, ovum, embryonic cell or embryo; 4. selectively lysing the at least one oocyte, blastocyst, ovum, embryonic cell or embryo of step 3 in the presence of spermatozoa so as to selectively release cellular material from the at least one lysed oocyte, blastocyst, ovum, embryonic cell or embryo; and 5. using the released cellular material in at least one downstream application.

### RELATED APPLICATIONS

This application claims priority of U.S. Patent Application Nos. 62/594,124 and 62/594,153, both filed on 4 Dec. 2017, the entire contents of which are each incorporated herein by way of cross-reference.

### TITLE 1

### Reproduction Methodologies

### Technical Field 1

This invention generally relates to assisted reproductive methodologies. In one aspect the invention concerns a method of producing at least one fertilised oocyte, pre-implanted embryo or blastocyst, preferably a pre-implanted fertilised oocyte, pre-implanted embryo or blastocyst in pig.

### Background Art 1

During the last three decades there has been considerable interest to improve in vitro production (IVP) of pigs not only for studying reproductive physiology but as part of other biotechnological and biomedical research, e.g. for production of transgenic pigs (Gil et al., 2010; Vajta and Callesen, 2012; Liu et al., 2011; Lopes et al., 2007; Li et al., 2013). An acceptable level (30-35%) of blastocyst production has been obtained in some laboratories (Kim et al., 2008; Somfai et al., 2005), but the variation and vulnerability of the porcine IVP system compared to, for example, bovine clearly illustrates that key factors remain to be solved.

Appropriate oocyte maturation is a prerequisite of successful fertilization and temporary blocking of meiotic maturation allows optimal cytoplasmic maturation, facilitating greater capacity and subsequent embryonic development (Fouladi-Nashta et al., 1998). Efforts have been made to reduce variation between fresh and frozen thawed semen for in vitro fertilization (IVF) results, but the efficiency of IVF still remains at 40-50% in most IVF laboratories (Li et al., 2003; Alminana et al., 2005), and the optimal dose of semen is still not understood. Despite attempts to use different types of media supplements and media conditions to improve IVP system, the success rate is still suboptimal. Traditional WP systems utilize several numbers of media from oocyte collection to embryo culture, which potentially leads to the variation and lack of reproducibility of the system.

Since there is no consistency between IVP protocols utilized by the different laboratories, an optimized IVP system is probably far from being realized.

### SUMMARY OF THE INVENTION 1

In one aspect of the invention, the inventors have developed a culture system for improved in vitro production (IVP) of fertilized oocytes, pre-implanted embryos or blastocysts.

In another aspect, the inventors have developed a simplified IVP method by using essentially a single culture medium, for the improved production of fertilized oocytes, pre-implanted embryos or blastocysts.

In another aspect, this improved embryo culture medium and method can be used as a standard across different laboratories with less variation, resulting in high reproducibility.

In another aspect, the simplicity of the inventor's IVP system facilitates high throughput without compromising embryonic development and quality, with the possibility of further application, for example, in biomedical research.

According to a first aspect of the present invention, there is provided a method of producing at least one fertilised oocyte, pre-implanted embryo or blastocyst, said method comprising the sequential steps of (1) washing, (2) collecting, (3) culturing, (4) fertilizing, optionally (5) washing, and optionally (6) culturing at least one oocyte in essentially the same culture medium except that culture medium's composition is altered step-wise by way of being selectively supplemented in one or more of steps (1) to (6) with at least one supplement, thereby improving production of said at least one fertilised oocyte or pre-implanted embryo or blastocyst.

Preferably, there is provided a method of producing at least one fertilised oocyte, pre-implanted embryo or blastocyst, said method comprising the sequential steps of (1) washing of cumulus-oocyte complexes (COCs) to release at least one oocyte from the COCs, (2) collecting the at least one oocyte of step (1), (3) culturing the at least one oocyte of step (2), (4) fertilizing the at least one oocyte of step (3) with sperm to produce at least one fertilised oocyte, embryo or blastocyst, optionally (5) washing at least one fertilised oocyte or embryo, and optionally (6) culturing the at least one fertilised oocyte or embryo from step (5) for further embryonic development, wherein essentially the same culture medium is used in steps (1) to (6) except that the culture medium's composition is altered step-wise by way of being selectively supplemented in one or more of steps (1) to (6) with at least one supplement, thereby improving production of said at least one fertilised oocyte, pre-implanted embryo or blastocyst.

According to a second aspect of the present invention, there is provided a method of producing at least one fertilised oocyte, pre-implanted embryo, or blastocyst, said method comprising the steps of:
(1) washing a preparation of cumulus-oocyte complexes (COCs) with culture medium twice and once with culture medium+supplement 1 to release at least one oocyte from the COCs;
(2) collecting the at least one oocyte of step (1) and culturing the at least one oocyte in culture medium+supplement 1 for a predetermined period of time;
(3) culturing the at least one oocyte of step (2) in culture medium+supplement 2 for a predetermined period of time;
(4) fertilising the at least one oocyte of step (3) with sperm in culture medium+supplement 3 for a predetermined period of time to produce at least one fertilised oocyte; optionally
(5) washing the at least one fertilised oocyte or resultant embryo with the culture medium+supplement 4; and optionally
(6) culturing the at least one fertilised oocyte or embryo from step (5) in the culture medium+supplement 4 for further embryonic development,
wherein essentially the same culture medium is used in each of steps (1) to (6).

According to a third aspect of the present invention, there is provided at least one fertilised oocyte, blastocyst or pre-implanted embryo when produced by the method of the first or second aspect.

According to a fourth aspect of the present invention, there is provided a culture medium or culture medium plus+supplement (1, 2, 3 or 4) as described in the first or second aspect of the present invention.

According to a fifth aspect of the present invention, there is provided a high throughput in vitro production (IVP) method for producing at least one pre-implanted fertilised oocyte, blastocyst or embryo as described according to any one of the earlier aspects of the present invention.

The oocyte can be sourced from a human or any suitable type of animal. The animal can be a mammal. The animal can be a farm animal such as a pig, cow, horse, sheep or goat. The animal can be a companion animal such as a dog or cat. The animal can be a laboratory animal such as a rabbit, mouse or rat. Preferably the oocyte is sourced from a pig.

Preferably more than one fertilised oocyte, embryo or blastocyst is produced by the different aspects of the invention described above. More preferably, many fertilised oocytes, blastocysts, or embryos are produced at the one and the same time.

Any suitable type of culture medium can be used. Although the inventors have found in vitro maturation (IVM) medium M-199 to be a particularly suitable medium for the first, second and fourth aspects of the invention, any medium having similar properties to M-199 can be used.

The culture medium can have one or more of the following types of ingredients: nutrition or energy (eg. glucose, protein, amino acids); minerals (eg. sodium chloride, calcium chloride, potassium chloride, monopotassium phosphate, sodium dihydrogen phosphate monohydrate, magnesium sulfate heptahydrate, sodium bicarbonate, sodium lactate, calcium lactate, iron nitrate nonahydrate); vitamins (eg. Vitamin A, Thiamine, Riboflavin, Pyridoxine, Pyridoxal HCl, PABA, Niacinamide, Niacin, Ascorbic acid, Biotin, D-calcium pantothenate, Choline chloride, Ergocalciferol, Folic acid, i-inositol, Menadione); pH Indicator (eg. phenol red); antimicrobials (eg. Penicillin G, Streptomycin); antioxidants (eg. glutathione); and other suitable supplements (eg. Porcine follicular fluid (PFF), Minimum essential medium, Foetal calf serum, Pregnant mare serum gonadotrophin, Human chorionic gonadotrophin, sodium pyruvate and caffeine).

Typically, M-199 will have the properties shown in Table 1A below.

TABLE 1A
Properties of M-199.

| | | | |
|---|---|---|---|
| | Molecular | Concentration | |
| Components | Weight | (mg/L) | mM |

Amino Acids

| | | | |
|---|---|---|---|
| Glycine | 75.0 | 50.0 | 0.6666667 |
| L-Alanine | 89.0 | 25.0 | 0.28089887 |
| L-Arginine hydrochloride | 211.0 | 70.0 | 0.33175355 |
| L-Aspartic acid | 133.0 | 30.0 | 0.22556391 |
| L-Cysteine hydrochloride-H₂O | 176.0 | 0.1 | 5.681818E−4 |
| L-Cystine 2HCl | 240.0 | 26.0 | 0.108333334 |
| L-Glutamic Acid | 147.0 | 75.0 | 0.5102041 |
| L-Glutamine | 146.0 | 100.0 | 0.6849315 |
| L-Histidine hydrochloride-H₂O | 210.0 | 21.88 | 0.10419047 |
| L-Hydroxyproline | 131.0 | 10.0 | 0.07633588 |
| L-Isoleucine | 131.0 | 40.0 | 0.3053435 |
| L-Leucine | 131.0 | 60.0 | 0.45801526 |
| L-Lysine hydrochloride | 183.0 | 70.0 | 0.38251367 |
| L-Methionine | 149.0 | 15.0 | 0.10067114 |
| L-Phenylalanine | 165.0 | 25.0 | 0.15151516 |
| L-Proline | 115.0 | 40.0 | 0.3478261 |
| L-Serine | 105.0 | 25.0 | 0.23809524 |
| L-Threonine | 119.0 | 30.0 | 0.25210086 |
| L-Tryptophan | 204.0 | 10.0 | 0.04901961 |
| L-Tyrosine disodium salt dehydrate | 261.0 | 58.0 | 0.22222222 |
| L-Valine | 117.0 | 25.0 | 0.21367522 |

Vitamins

| | | | |
|---|---|---|---|
| Ascorbic Acid | 176.0 | 0.05 | 2.840909E−4 |
| Biotin | 244.0 | 0.01 | 4.0983607E−5 |
| Choline chloride | 140.0 | 0.5 | 0.0035714286 |
| D-Calcium pantothenate | 477.0 | 0.01 | 2.096436E−5 |
| Folic Acid | 441.0 | 0.01 | 2.2675737E−5 |
| Menadione (Vitamin K3) | 172.0 | 0.01 | 5.8139532E−5 |
| Niacinamide | 122.0 | 0.025 | 2.0491803E−4 |
| Nicotinic acid (Niacin) | 123.0 | 0.025 | 2.0325204E−4 |
| Para-Aminobenzoic Acid | 137.0 | 0.05 | 3.6496352E−4 |
| Pyridoxal hydrochloride | 204.0 | 0.025 | 1.2254903E−4 |
| Pyridoxine hydrochloride | 206.0 | 0.025 | 1.21359226E−4 |
| Riboflavin | 376.0 | 0.01 | 2.6595744E−5 |
| Thiamine hydrochloride | 337.0 | 0.01 | 2.967359E−5 |
| Vitamin A (acetate) | 328.0 | 0.1 | 3.0487805E−4 |
| Vitamin D2 (Calciferol) | 397.0 | 0.1 | 2.5188917E−4 |
| alpha Tocopherol phos. Na salt | 554.7 | 0.01 | 1.8027762E−5 |
| i-Inositol | 180.0 | 0.05 | 2.7777778E−4 |

Inorganic Salts

| | | | |
|---|---|---|---|
| Calcium Chloride (CaCl₂) (anhyd.) | 111.0 | 200.0 | 1.8018018 |
| Ferric nitrate (Fe(NO₃)—9H₂O) | 404.0 | 0.7 | 0.0017326733 |
| Magnesium Sulfate (MgSO₄) (anhyd.) | 120.0 | 97.67 | 0.8139166 |
| Potassium Chloride (KCl) | 75.0 | 400.0 | 5.3333335 |
| Sodium Bicarbonate (NaHCO₃) | 84.0 | 2200.0 | 26.190475 |
| Sodium Chloride (NaCl) | 58.0 | 6800.0 | 117.24138 |
| Sodium Phosphate monobasic | 138.0 | 140.0 | 1.0144928 |
| (NaH₂PO₄—H₂O) | | | |

Other Components

| | | | |
|---|---|---|---|
| 2-deoxy-D-ribose | 134.0 | 0.5 | 0.0037313432 |
| Adenine sulfate | 404.0 | 10.0 | 0.024752475 |
| Adenosine 5′-phosphate | 347.0 | 0.2 | 5.763689E−4 |
| Adenosine 5′-triphosphate | 605.0 | 1.0 | 0.0016528926 |
| Cholesterol | 387.0 | 0.2 | 5.1679584E−4 |
| D-Glucose (Dextrose) | 180.0 | 1000.0 | 5.5555553 |
| Glutathione (reduced) | 307.0 | 0.05 | 1.6286645E−4 |
| Guanine hydrochloride | 188.0 | 0.3 | 0.0015957447 |
| Hypoxanthine Na | 136.0 | 0.4 | 0.0029411765 |
| Phenol Red | 376.4 | 20.0 | 0.053134963 |
| Ribose | 150.0 | 0.5 | 0.0033333334 |
| Sodium Acetate | 82.0 | 50.0 | 0.6097561 |
| Thymine | 126.0 | 0.3 | 0.0023809525 |
| Tween 80 ® | | 20.0 | Infinity |
| Uracil | 112.0 | 0.3 | 0.0026785715 |
| Xanthine-Na | 152.0 | 0.34 | 0.0022368422 |

References: Morgan, J. F. and Campbell, M. E. (1955) J. Natl. Cancer Inst., 16:557; Morgan, J. F., Morton, H. J. and Parker R. C. (1950) Proc. Soc. Exp. Biol. Med., 73:1

Other suitable media—that is, any medium having similar characteristics to M-199—can be characterised as having the general properties shown in Table 2A below.

TABLE 2A
General properties of suitable culture media other than M-199.

| | | | Specific |
|---|---|---|---|
| | | | example and |
| | Concentration | | concentration |
| Ingredient type | range | | range |
| Nutrients: | | | |
| Energy source: | 2.5-10.5 | mM | 5.5 mM |
| Glucose | | | |
| Protein/amino acid source: | 0.049-0.684 | mM | See |
| Cystine, Glutamine, Alanine, | | | Table 1A |
| Arginine, Aspartic acid, Glutamic | | | |
| acid, Glycine, Histidine, Hydroxy | | | |
| L-proline, Isoleucine, Leucine, | | | |
| Lysine, Methionine, Phenylalanine, | | | |
| Serine, Threonine, Tryptophan, | | | |
| Tyrosine, Valine | | | |
| Mineral source: | 0.001-117.24 | mM | See |
| Sodium chloride, Calcium chloride, | | | Table 1A |
| Potassium chloride, Monopotassium | | | |
| phosphate, Sodium dihydrogen | | | |
| phosphate monohydrate, Magnesium | | | |
| sulfate heptahydrate, Sodium | | | |
| bicarbonate, Sodium lactate, Calcium | | | |
| lactate, Iron Nitrate nonahydrate | | | |
| Vitamin source: | 0.003-6.81 | mM | See |
| Vitamin A, Thiamine, Riboflavin, | | | Table 1A |
| Pyridoxine, Pyridoxal HCl, PABA, | | | |
| Niacinamide, Niacin, Ascorbic acid, | | | |
| Biotin, D-calcium pantothenate, | | | |
| Choline chloride, Ergocalciferol, | | | |
| Folic acid, i-inositol, Menadione | | | |
| pH Indicator: | 0.02-0.08 | mM | 0.053 mM |
| Phenol red | | | |
| Antimicrobial: | 50-150 | IU/ml | 100 IU/ml |
| Penicillin G | 25-100 | IU/ml | 50 IU/ml |
| Streptomycin | | | |
| Antioxidant: | 0.8-2.0 | mM | 1.62M |
| Glutathione | | | |

Any suitable type of supplement can be used. Preferably, each supplement helps the supplemented culture medium to mimic or simulate or be more chemically similar to the oviduct environment from which the oocyte was obtained. One or more of the supplements can comprise a hormone, growth promoter and/or an energy provider (eg. cAMP). If the oocyte is sourced from a pig, then porcine-derived molecules can be used, but this need not necessarily be the case. In some instances, porcine-compatible molecules (synthetic or sourced from a different species) can be used.

For example, the supplement of the washing step can comprise a hormone and growth promoter, the supplement of the collecting step can comprise a hormone, growth promoter and energy provider, the supplement of the culturing step can comprise a growth promoter, the supplement of the fertilisation step can comprise an energy provider, the supplement of the washing step can comprise a growth promoter, and/or the supplement of the culturing step can comprise a growth promoter.

Further preferred properties of the + supplements mentioned in this specification are shown in Table 3A below.

TABLE 3A
Properties of +supplements.

| | | | Specific | |
|---|---|---|---|---|
| | | | example and | |
| | Concentration | | concentration | |
| Ingredient type | range | | range | |
| +supplement 1 | | | | |
| Porcine follicular fluid (PFF) | 5-25% | v/v | 10-15% | v/v |
| Pregnant mare serum gonadotrophin | 10-60 | IU/ml | 20-30 | IU/ml |
| (PMSG) | | | | |
| Human chorionic gonadotrophin (hGC) | 5-30 | IU/ml | 10-15 | IU/ml |
| cAMP (optional if ooctyes obtained | 5-30 | IU/ml | 1 | mM |
| from gilts) | 0.1-10 | mM | | |
| +supplement 2 | | | | |
| Porcine follicular fluid (PFF) | 5-30% | v/v | 10-15% | v/v |
| cAMP (optional if oocytes obtained | 0.1-10 | mM | 1-2 | mM |
| from gilts) | | | | |
| +supplement 3 | | | | |
| Sodium pyruvate | 1-10% | v/v | 2-3% | v/v |
| Caffeine | 0.1-10 | mM | 2-3 | mM |
| Glucose | 2.5-10.5 | mM | 5.5 | mM |
| CaCl₂ | 2-10 | mM | 5-7 | mM |
| NaHCO₃ | 15-30 | mM | 25-27 | mM |
| BSA | 0.1-0.5% | | 0.1-0.2% | |
| +supplement 4 | | | | |
| Porcine follicular fluid (PFF) | 5-30% | v/v | 10-15% | v/v |

| | | |
|---|---|---|
| Bovine serum albumin (BSA) | 0.2-0.9% | 0.3-0.4% |

Reference: Eagle, H. (1959) Science 130:432.

In step (1), the preparation of COCs can be of any suitable form. Preferably COCs with uniform ooplasm and at least two layers of compact cell mass are used. Preferably pig COCs are used.

In step (1), the washing of COCs to obtain at least one oocyte can be carried out in any suitable way. Preferably the COCs are washed twice in culture medium+supplement 1. Culture medium+supplement 1 can be as described in the Tables. Preferably supplement 1 comprises 15% (v/v) porcine follicular fluid (PFF), 15 IU/ml human chorionic gonadotrophin (hCG), and 30 IU/ml pregnant mare serum gonadotrophin (PMSG).

PFF can be collected from superficial follicles, approximately 3-6 mm in diameter. Collected PFF can be filtered using a 0.20 μm syringe filter and stored at −20° C. until used.

The method can comprise the step of collecting ovaries prior to step (1). Ovaries can be collected from slaughtered pre-pubertal gilts and sows in any suitable way.

The ovaries can be stored in a salt solution, eg. in about 0.9% NaCl solution at 30-38° C., until processed according to step (1)

Prior to carrying out step (2), COCs can be aspirated from about 3-8 mm diameter follicles and allowed to settle down as sediment at 38° C. for few minutes.

In step (2) the least one oocyte can be collected in any suitable way. Typically this will involve a transfer pipette, with collection carried out with the aid of a microscope.

In step (2) any suitable number of oocytes can be collected for culturing. Preferably between about 5 and 200 oocytes, and more preferably between about 10 and 70 oocytes, and even more preferably between about 25 and 30 oocytes are collected for culturing.

Culture medium+supplement 1 can be as described in the Tables. Preferably supplement 2 comprises 15% (v/v) porcine follicular fluid (PFF), 15 IU/ml human chorionic gonadotrophin (hCG), and 30 IU/ml pregnant mare serum gonadotrophin (PMSG).

For oocytes retrieved from gilts, cAMP (for energy) can be added to the IVM medium at a concentration of about 1 mM.

In step (2) the at least one oocyte can be cultured in any suitable way. Typically, this would involve culturing at least one oocyte in an incubator at 38° C., such as in a carbon dioxide incubator. This can involve placing at least one oocyte in a small volume of culture medium+supplement 1, and covering it with oil to prevent evaporation, such as warm paraffin oil.

In step (2) the least one oocyte can be cultured for any suitable period of time. Preferably the predetermined period of time is approximately 22 hours, although the predetermined period time could be approximately 10, 11, 12, 13, 14, 15, 16, 17, 18, 19, 20, 21, 22, 23, 24, 25, 26, 27, 28, 29 or 30 hours (or even shorter or longer).

After step (2), the culture medium+supplement 1 can be removed and replaced with culture medium+supplement 2, thereby removing the two gonadotrophins. For oocytes retrieved from gilts, cAMP can be added to the IVM medium at a concentration of 1 mM.

Culture medium+supplement 2 can be as described in the Tables. Preferably supplement 2 comprises 1 mM cAMP and 15% (v/v) porcine follicular fluid (PFF).

In step (3) the at least one oocyte can be cultured in any suitable way. Typically this would involve culturing the at least one oocyte as described for step (2), for the predetermined period of time as described for step (2).

In step (4) the least one oocyte can be fertilised with sperm/spermatozoa in any suitable way to produce at least one fertilised oocyte, blastocyst or embryo.

In step (4) the least one oocyte can be incubated with sperm for any suitable period of time so as to produce at least one fertilised oocyte, blastocyst or embryo. Preferably the predetermined period of time is approximately 3.5 to 4 hours, although the predetermined period time could be approximately 0.5, 1, 1.5, 2, 2.5, 3, 3.5, 4, 4.5, 5, 5.5, 6, 6.5, 7, 7.5 or 8 hours (or even shorter or longer).

Culture medium+supplement 3 can be as described in the Tables. Preferably supplement 3 comprises 2 mM sodium pyruvate, 2 mM caffeine, 6 mM CaCl₂, 13 mM NaHCO₃, 0.1% BSA and 5.5 mM glucose. For oocytes retrieved from gilts, cAMP can be included at a concentration of 1 mM.

The method can include a step of preparing sperm for fertilization in step (4). Collected semen or collected diluted semen can be centrifuged (eg. at 1500 rpm for 5 min) in a tube to remove the supernatant. The resultant sperm pellet can optionally be washed with phosphate-buffered saline (PBS) and re-centrifuged. The resultant sperm pellet can optionally be washed with culture medium+supplement 3 as described in the Tables. After removing the supernatant, the sperm pellet can be re-suspended in culture medium+supplement 3. The tube can then be placed at a 45° angle in an incubator for 30 min before the sperm is used. Sperm concentration can be determined using a haemocytometer.

Any suitable number of sperm/spermatozoa can be used in step 4. This can depend on the total number of oocytes. Preferably, approximately 60,000 spermatozoa can be used to inseminate approximately 20-30 oocytes, being a ratio of about 3000:1.

The method can include a step of denuding the at least one oocyte and this can be carried out in any suitable way. For example, oocytes can be denuded by repetitive gentle pipetting of oocytes in a small volume of culture medium. Denuding can be performed after culture step (3), eg. after about 44 h of maturation culture before IVF step (4).

In step (5), the washing of the at least one fertilised oocyte (or resultant blastocyst or embryo) can be carried out in any suitable way. Preferably the at least one fertilised oocyte is washed in culture medium+supplement 4. Culture medium+supplement 4 can be as described in the Tables. Preferably supplement 4 comprises 15% (v/v) porcine follicular fluid (PFF) and 0.4% BSA. For example, fertilized oocytes can be washed twice with culture medium+supplement 4.

In step (5) the at least one fertilised oocyte (or embryo) can be cultured in any suitable way. Typically this would involve culturing the at least one fertilised oocyte in an incubator at 37° C. or 38.5° C., such as in a carbon dioxide incubator. This can involve placing the least one fertilised oocyte in culture medium+supplement 4 as described in the Tables.

In step (5) the at least one fertilised oocyte (or embryo) can be cultured for any suitable period of time. Preferably the predetermined period of time is approximately 1, 2, 3, 4, 5, 6, 7, 8, 9, 10, 11, 12, 13, 14 or 15 days (or even longer or shorter). Preferably the predetermined period of time is approximately 5 to 9 days, more preferably 7 days.

In some embodiments, embryos can be cultured in groups of about 25-30 per droplet in culture medium+supplement 4 for about 7 days at 38.5° C. in a CO₂ incubator. Cleavage and blastocyst formation of the fertilized oocytes can be the examined at 2 and 7 days after the start of culture, respectively. Embryos that developed into 2, 4, 8 or more cells can be used for further downstream processing.

General techniques and methodologies for oocyte recovery, in vitro maturation, sperm preparation, in vitro fertilization and in vitro culturing can be found in the following references, each of which is incorporated herein in its entirety by way of cross-reference (Bagg et al., 2006; Gil et al., 2008; Tanihara et al., 2013; Hossain et al., 2007; Nagai, 1996):

Any of the features described herein can be combined in any combination with any one or more of the other features described herein within the scope of the invention.

The reference to any prior art in this specification is not, and should not be taken as an acknowledgement or any form of suggestion that the prior art forms part of the common general knowledge.

### BRIEF DESCRIPTION OF DRAWINGS 1

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1A is a schematic representation of a simplified high-throughput IVP system utilising essentially a single culture medium (M-199), according to an embodiment of the invention.

### DETAILED DESCRIPTION OF EMBODIMENTS 1

### EXAMPLE 1

### Simplified IVP Method Essentially Using a Single Culture Medium for the Improved Production of Pig Embryos

This Example describes a simplified method of pre-implantation in-vitro pig embryo development suitable for use in a high-throughput IVP system.

Materials and Methods

All chemicals were purchased from Thermo Fisher Scientific except otherwise indicated. All in-vitro cultures were made with an overlay of 400 μl mineral oil in each culture well. The basic culture medium used in this Example was M-199. In 1950, Morgan first proposed a chemically defined nutritional source for cell cultures without using animal products and/or tissue extracts. The advantage of using this medium is the scope of its diverse ingredients to support oocyte and embryo development at different developmental stage.

Oocyte Recovery and In Vitro Maturation

Ovaries were collected from slaughtered pre-pubertal gilts and sows from a nearby abattoir (Highchester Meats Pty Ltd, Gleneagle, Australia) and transported to the laboratory in 0.9% NaCl solution at 30-38° C. Cumulus-oocyte complexes (COCs) were aspirated from 2-8 mm diameter follicles and allowed to settle down as sediment at 38° C. for few minutes. COCs with uniform ooplasm and at least two layers of compact cell mass were washed twice in in vitro maturation (IVM) medium (M-199) and once in M-199 supplemented with 15% (v/v) porcine follicular fluid (PFF), 1 mM cAMP, 15 IU/ml human chorionic gonadotrophin (hCG) (Intervet, Holland), and 30 IU/ml pregnant mare serum gonadotrophin (PMSG) (Intervet, Holland). PFF was collected from superficial porcine follicles, 3-6 mm in diameter. Thereafter, PFF was filtered using 0.20 μm syringe filters and stored at −20° C. until used. About 25-30 oocytes were placed in each droplet of 400 μl IVM medium in petri dishes and covered with warm paraffin oil and cultured for 22 h at 38° C. in a CO₂ incubator. After the 22 h culture, the medium was removed and replaced with fresh IVM medium containing the two gonadotrophins, and then cultured for an additional 22 hours. For oocytes retrieved from gilts, cAMP was added to the IVM medium at a concentration of 1 mM.

Sperm Preparation and In-Vitro Fertilization

Diluted semen was collected from a nearby AI Company (Premier Genetics, Wacol, Australia) and transported to the laboratory immediately in an insulated box at approximately 38° C. The sperm was mixed gently and was poured into a 2-ml centrifuge tube to just under the top of the tube before being centrifuged at 1500 rpm for 5 min to remove the supernatant. Sperm were then re-suspended in 2 ml IVF medium (M-199 supplemented with 2 mM caffeine, 2 mM Na-pyruvate, 505 mM glucose, 0.1% BSA, 6 mM CaCl₂, and 26 mM NaHCO₃) and washed twice with centrifugation as before. After removing the supernatant, the sperm pellet was re-suspended in IVF medium. The tubes were then placed at a 45° angle in the incubator for 30 min before the sperm was used. Sperm concentration was determined using a Neubauer haemocytometer. A total of 60,000 spermatozoa was used to inseminate 20-30 oocytes in a droplet and co-incubated for 4-6 h to complete the fertilization.

In Vitro Culture

After completion of IVF, fertilized oocytes were washed twice with in vitro culture (IVC) medium (M-199 supplemented with 15% porcine follicular fluid). Embryos were cultured in groups of 25-30 per droplet in petri dishes in 400 μl culture media for 7 days at 38.5° C. in a CO₂ incubator. Cleavage and blastocyst formation of the oocytes was examined at 2 and 7 days after the start of culture, respectively. Embryos that developed into 2, 4, 8 or more cells, were transferred directly in 2 μl volumes to 200 μl PCR tubes for further processing.

Results

The influence of simplified (one basic IVM medium, M-199) on pre-implantation embryo development is shown in Table 4A.

TABLE 4A
Step by step mean development of in vitro sow and gilt embryos
using the simplified IVP system with supplements.

| Source | Number | Cleaved | 8-cell | Blastocyst |
|---|---|---|---|---|
| of | of | (% of | and above | (% of |
| oocytes | oocyte | oocytes) | (% of oocytes) | oocytes) |
| Sow | 545 | 382 (70.0) | 299 (54.8) | 224 (41.1) |
| Gilt | 463 | 247 (53.3) | 131 (28.29) | ND |

After adjusting NaHCO₃, CaCl₂ and BSA levels
in the IVF media, embryo production changed

| | | | | |
|---|---|---|---|---|
| Gilt | 1240 | 834 (67.2) | 425 (34.3) | ND |
| Mean | 2248 | 1463 (65.0) | 855 (38.0) | ND |

A significant level of embryo development was found in both sow and gilts, and the mean cleavage rate was 65%. With adjustment of NaHCO₃, CaCl₂ and BSA levels in the IVF media, embryo production changed. Cleavage rates changed from 62% on average, to 67%. The cleavage rate was lower in gilts compared to sows, and consequently the rate of blastocyst development was much lower in gilts, though in most cases embryos were not assessed up to blastocyst stage. A substantial number of embryos was observed to pass beyond the 8-cell stage in all experiments, averaging ˜38%. The average blastocyst development was ˜41% which is still consistent with the number of embryos cleaved and passed 8-cell stage.

Addition of CaCl₂, NaHCO₃ and BSA in the IVF medium resulted in higher cleavage rate and subsequent development which made overall embryo production more stable.

Chemical compositions of commonly used in vitro maturation (IVM) media are presented in Table 5A.

TABLE 5A
Comparison of chemical compositions of different
in vitro maturation medium for pig

| | IVM media (in mM) | | | |
|---|---|---|---|---|
| Ingredients | M-199 | TCM-199 | NCSU-23 | mWM |
| NaCl | 117.24 | 116.36 | 108.73 | 68.49 |
| KCl | 5.33 | 5.36 | 4.78 | 4.78 |
| CaC | 1.80 | 1.80 | 1.70 | — |
| Fe (NO₃)₃•9H₂O | 0.001 | — | — | — |
| KHPO₄ | — | — | 1.19 | 1.19 |
| NaHPO₄•H₂O | 1.014 | — | — | — |
| MgSO₄•7H₂O | 0.81 | 0.81 | 1.19 | 1.19 |
| NaHCO₃ | 26.19 | 26.19 | 25.07 | 25.07 |
| Glucose | 5.55 | 5.55 | 5.55 | 5.56 |
| Glutathione (reduced) | 1.62 | — | — | — |
| Phenol red, Na | 0.053 | — | — | — |
| Sodium lactate | — | — | — | 25.20 |
| Sodium pyruvate | — | — | — | 0.33 |
| Calcium lactate | — | — | — | 1.71 |
| Glutamine | 0.684 | 0.68 | 1.0 | — |
| Taurine | — | — | 7.0 | — |
| Hypotaurine | — | — | 5.0 | — |
| Penicillin G (IU/ml) | — | 100 | 100 | 100 |
| Streptomycine (IU/ml) | — | 50 | 50 | 50 |
| PFF (% v/v) | — | 10 | 10 | 10 |
| BSA (mg/ml) | — | — | — | — |
| Cystine | 0.108 | 0.57 | 0.57 | 0.57 |
| Ascorbic acid | 2.84 | — | — | — |
| Biotin | 4.09 | — | — | — |
| D-calcium pantothenate | 2.09 | — | — | — |
| Choline chloride | 0.003 | — | — | — |
| Ergocalciferol | 2.51 | — | — | — |
| Folic acid | 2.26 | — | — | — |
| i-inositol | 2.77 | — | — | — |
| Menadione | 5.81 | — | — | — |
| Niacin | 2.03 | — | — | — |
| Niacinamide | 2.04 | — | — | — |
| PABA | 3.649 | — | — | — |
| Pyridoxal HCl | 1.225 | — | — | — |
| Pyridoxine HCl | 1.213 | — | — | — |
| Riboflavin | 2.659 | — | — | — |
| Thiamine HCl | 2.967 | — | — | — |
| Vitamin A acetate | 3.048 | — | — | — |
| L-alanine | 0.280 | — | — | — |
| L-arginine | 0.331 | — | — | — |
| L-aspartic acid | 0.225 | — | — | — |
| L-glutamic acid | 0.510 | — | — | — |
| Glycine | 0.666 | — | — | — |
| L-histidine | 0.104 | — | — | — |
| Hydroxy L-proline | 0.347 | — | — | — |
| L-isoleucine | 0.305 | — | — | — |
| L-leucine | 0.458 | — | — | — |
| L-lysine | 0.382 | — | — | — |
| L-methionine | 0.100 | — | — | — |
| L-phenyl alanine | 0.151 | — | — | — |
| L-serine | 0.238 | — | — | — |
| L-threonine | 0.252 | — | — | — |
| L-tryptophan | 0.049 | — | — | — |
| L-tyrosine | 0.222 | — | — | — |
| L-valine | 0.213 | — | — | — |

Discussion

The main finding of this Example is presented in Table 4A, where the resulting blastocyst rate is ˜41% with a reasonably low variation. Irrespective of the source of oocytes, cleavage rate of embryos was much higher (˜65%) than the average of other laboratories (˜50%). We believe that this outcome is due to the step-wise adjustment of the procedure, with the use of single basic common medium for every culture step as the main effect. The present study also demonstrated that the IVM culture medium used (M-199 with its different supplements) and procedure followed in this study is ideally suitable for the IVP of sow embryos; as well as for gilts, however for gilts it might need further fine tuning. One possible option for gilts is supplementing cAMP in the oocyte IVM medium. Oocytes derived from adult sows have a higher cAMP content than that obtained from gilts (Bagg et al., 2006), and cAMP treatment enhances the developmental capacity of gilt oocytes by transiently elevating their cAMP content. During the time of oocyte processing, both sperm and oocytes produce ROS, addition of bicarbonate can compensate that. We observed, when IVF medium was further supplemented with bicarbonate, calcium and BSA, cleavage rate and subsequent embryo development increased significantly.

Although many different specialized culture media (TCM-199, NCSU-23, PZM-3, SOF) are available for pig embryo manipulations, few of them produce consistent results and hence are not reproducible. Since pig embryos are usually processed by most laboratories in 4 or 5 different media, we hypothesise that this causes stress for the oocytes/embryos (when adjusting to the changing chemical environments) and results in poor yields which is inconsistent. On the other hand, M-199 medium is a widely used cell culture medium, though to date it has limited use in mammalian embryo culture, particularly in pigs.

In conclusion, the pig IVP method used in the present study may play a crucial role in commercial production and future pig embryo research. The simplicity of the proposed method may facilitate its application in a high-throughput IVF system and as a standard for IVP laboratories.

### REFERENCES 1

Almiñana, C., Gil, M. A., Cuello, C., Roca, J., Vazquez, J. M., Rodriguez-Martinez, H., Martinez, E. A., 2005.Adjustments in IVF system for individual boars: value of additives and time of sperm-oocyte co-incubation. Theriogenology 64:1783-1796.

Bagg, M. A., Nottle, M. B., Grupen, C. G., Armstrong, D. T., 2006. Effect of dibutyrylcAMP on the cAMP content, meiotic progression, and developmental potential of in vitro matured pre-pubertal and adult pig oocytes. Mol Reprod Dev 73: 1326-1332.

Fouladi-Nashta, A. A., Waddington, D., Campbell, K. H. S., 1998, Maintenance of bovine oocytes in meiotic arrest and subsequent development in vitro. A comparative evaluation of antral follicle culture with other methods. Biol Reprod 59: 255-262.

Gil, M. A., Cuello, C., Parrilla, I., Vazquez, J. M., Roca, J., Martinez, E. A., 2008. Advances in swine in vitro embryo production technologies. Reprod Dom Anim 45: 40-48.

Kim, J. S., Cho, Y. S., Song, B. S., Wee, G., Park, J. S., Choo, Y. K., Yu, K., Lee, K. K., Han, Y. M., Koo, D. B., 2008. Exogenous dibutyrylcAMP affects meiotic maturation via protein kinase A activation; it stimulates further embryonic development including blastocyst quality in pigs. Theriogenology 69: 290-301.

Li, R., Liu, Y., Pedersen, H. S., Kragh, P. M., Callesen, H., 2013. Development and quality of porcine parthenogenetically activated embryos after removal of zonapellucida. Theriogenology 80:58-64.

Liu, Y., Østrup, O., Li, J., Vajta, G., Kragh, P. M., Purup, S., Callesen, H., 2011. Cell colony formation induced by Xenopus egg extract as a marker for improvement of cloned blastocyst formation in the pig. Cell Reprogram. 13:521-526.

Lopes, A. S., Wrenzycki, C., Ramsing, N. B., Herrmann, D., Niemann, H., Løvendahl, P., Greve, T., Callesen, H., 2007. Respiration rates correlate with mRNA expression of G6PD and GLUT1 genes in individual bovine in vitro-produced blastocysts. Theriogenology 68:223-236.

Somfai, T., Kikuchi, K., Onishi, A., Iwamoto, M., Fuchimoto, D., Papp, A. B., et al.,2003. Meiotic arrest maintained by cAMP during the initiation of maturation enhances meiotic potential and developmental competence and reduces polyspermy of IVM/IVF porcine oocytes. Zygote 11: 199-206.

Tanihara, F., Nakai, M., Kaneko, H., Noguchi, J., Otoi, T., Kikuchi, K., 2013. Evaluation of zonapellucidafunction for sperm penetration during in vitro fertilization in pigs. J Reprod Dev. doi.org/10.1262/jrd.2013-021.

Hossain, M. S., Tareq, K. M. A., Hamano, K. and Tsujii, H. (2007): The effect of the fatty acids on boar sperm motility, viability and acrosome reaction. Reproductive Medicine and Biology 6, 235-239.

Nagai T., 1996. In vitro maturation and fertilization of pig oocytes. Anim Reprod Sci, 42: 153-163.

Vajta, G., Callesen, H., 2012. Establishment of an efficient somatic cell nuclear transfer system for production of transgenic pigs. Theriogenology 77:1263-1274.

### TITLE 2

### Lysis Method

### Technical Field 2

This invention generally relates to selectively lysing an oocyte, blastocyte/blastocyst, ovum, embryonic cell or embryo in the presence of spermatozoa. In a preferred embodiment, the invention relates to selectively lysing an oocyte, blastocyst, ovum, embryonic cell or embryo in the presence of spermatozoa such that genetic material and other cellular contents are released from the lysed oocyte, blastocyst, ovum, embryonic cell or embryo alone, while keeping spermatozoa intact.

### Background Art 2

Changes to an oocyte or embryo's genetic make-up can come from various sources and it is desirable to be able to identify and characterise those changes. For example, techniques such as gene editing (Clustered Regulatory Interspaced Short Palindromic Repeats (CRISPR) and Transcription Activator-Like Effector Nucleases (TALEN)) can be used to intentionally modify the oocyte/embryo genome, and it is desirable to be able to confirm that only the intended gene edits have been made. Semen treatments can be used to test mutagenic effects of chemicals on embryos, and it is desirable to be able to identify any and all changes made to the genome. Sex selection treatments of semen (flow cytometry, micro fluidic devices, antibody treatments etc) as well as sex selection treatments of fertilized oocytes/embryos can potentially lead to genetic modification of the fertilized oocytes/embryo, and it is desirable to be able to identify any and all changes made to the genome.

The polymerase enzyme, and more particularly the polymerase chain reaction (PCR) technique, has been used to identify and characterize genetic modifications to embryos. However, the technique has its challenges when used for this purpose. For example, due to the high sensitivity of the technique, the presence of non-embryonic DNA contaminants (such as from sperm or epithelial cells) can readily produce false results. For example, wash solutions, lysis solutions and suspension media for oocytes/embryos can lead to poor performance or complete inhibition of the PCR technique, or the introduction of contaminating genetic material. For example, excess handling/multistep handling of oocytes/embryos can lead to sub optimal genetic material for PCR amplification.

As mentioned above, when identifying and characterising potential changes to the genome of an embryo, the possibility of a false result needs to be carefully ruled out. For example, a false result may arise from contaminating DNA, from low levels of sperm DNA or male somatic cell DNA that could have been carried over after fertilisation of the ova.

A conventional technique for reducing DNA contamination/carry over involves completely removing the Zona Pellucida (ZP) by washing oocytes up to six or seven times (Pomp, 1995). The first wash is in Ca²⁺ and Mg²⁺ free Dulbecco's phosphate-buffered saline (DPBS). The oocytes are transferred to Tyrodes solution (137 mM NaCl, 2.7 mM KCl, 1 mM MgCl₂, 1.8 mM CaCl₂, 0.2 mM Na₂HPO₄, 12 mM NaHCO₃, 5.5 mM D-glucose) followed by four or more washes in Ca²⁺ and Mg²⁺ free DPBS. Oocytes are then stored at this stage. Prior to use in a PCR technique, oocytes are digested with Proteinase K overnight, followed by inactivation of Proteinase K at 98° C. for 10 min (Tor 2013).

A disadvantage of this multistep conventional technique is that it is tedious in that oocytes need to be repetitively picked up using a fine pipette under a stereo microscope and placed in the subsequent appropriate wash solution et cetera. This conventional technique does not lend itself to being developed into a high throughput assay. (A good high throughput assay is one that requires as few steps as possible, whilst at the same time yielding results that are robust and consistent.)

Selective lysis is another option for preventing contaminating DNA from being PCR amplified. Selective lysis methods are not new and have mainly been utilised in sexual assault samples where there is a need to distinguish between victim (epithelial cells) and spermatozoa (perpetrator cells) (Norris, 2007). The preferred methods currently utilised are variations of the procedure developed by Gill (1985) and Yoshida (1995). The samples are first treated by a lysis solution (TNE buffer: 10 mM Tris-HCl (pH 8.0), 10 mM ethylene diamine tetra-acetate (EDTA), 100 mM NaCl with 1% sodium dodecyl sulfate (SDS) and 100 pg/ml Proteinase K) at 70° C. for 1-3 hrs to lyse epithelial cells. This is repeated a few times to release epithelials from a matrix such as a cotton swab, for instance. The next step is using the same lysis buffer with 0.04 M dithiothreitol (DTT) added for lysing of spermatozoa for more than 8 h at 56° C. in a shaking water bath.

The problem with utilising this procedure in a high throughput in vitro fertilisation (HT IVF) system, is that, although capable of selectively lysing oocytes, the TNE lysis buffer for epithelial cells contains PCR inhibitors (Rossen, et al 1992). These need to be subsequently removed by phenol extraction and precipitation of the DNA with 3M sodium acetate and ice-cold absolute ethanol. Furthermore, the lysis procedure can take from 1-4 hrs.

The Single-cell REPLIg kit from Qiagen has been utilised for pre-amplification by PCR. The lysis buffer included with the kit, however, contains DTT and KOH, both components, while not PCR inhibitory, cause unwanted lysis of the accompanying spermatozoa. The Qiagen protocol requires the addition of a stop solution after a 10 minutes 65° C. incubation. This step requires additional handling with opening of tubes to introduce the stop solution with the danger of introducing contamination.

Apart from the genetic aspect, there are other reasons as to why it would be desirable to selectively lyse oocytes in the presence of spermatozoa or somatic cells. This is because oocytes contain other cellular materials of interest (eg. for downstream applications, particularly “Omics”: genomics, transcriptomics, epigenomics and proteomics [see Wang and Bodovitz, Trends Biotechnol. 2010 June; 28 (6): 281-290]). Epithelial cells are very friable in comparison with spermatozoa or oocytes. The relatively mild conditions used for lysis of somatic and epithelial cells are not suitable for lysing oocytes.

### SUMMARY OF THE INVENTION 2

In one aspect of the invention, the inventors have developed a lysis solution for selectively lysing an oocyte, blastocyte/blastocyst, ovum, embryonic cell or embryo in the presence of spermatozoa and for selectively releasing cellular material from only the lysed oocyte, blastocyst, ovum, embryonic cell or embryo.

In another aspect of the invention, the inventors have developed a lysis solution for selectively lysing an oocyte, blastocyte/blastocyst, ovum, embryonic cell or embryo in the presence of spermatozoa and for selectively releasing cellular material from only the lysed oocyte, blastocyst, ovum, embryonic cell or embryo such that the released cellular material within the lysis solution is compatible with a downstream application.

In yet another aspect of the invention, the inventors have developed a method of selectively lysing an oocyte, blastocyte/blastocyst, ovum, embryonic cell or embryo in the presence of spermatozoa such that cellular material from a lysed oocyte, blastocyst, ovum, embryonic cell or embryo and the lysis solution are compatible with a downstream application.

According to a first aspect of the present invention, there is provided a method of selectively lysing an oocyte, blastocyte/blastocyst, ovum, embryonic cell or embryo in the presence of spermatozoa, said method comprising the step of:

subjecting the oocyte, blastocyst, ovum, embryonic cell or embryo to a lysis solution such that the oocyte, blastocyst, ovum, embryonic cell or embryo is lysed and cellular material is released from the oocyte, blastocyst, ovum, embryonic cell or embryo, but such that the spermatozoa is not lysed.

According to a 2^{nd} aspect of the present invention, there is provided released cellular material when produced by the method of the 1^{st} aspect.

According to a 3^{rd} aspect of the present invention, there is provided a lysis solution for selectively lysing an oocyte, blastocyte/blastocyst, ovum, embryonic cell or embryo in the presence of spermatozoa that is not lysed by the lysis solution, and for selectively releasing cellular material from the lysed oocyte, blastocyst, ovum, embryonic cell or embryo such that the cellular material and the lysis solution are compatible with a downstream application.

Preferably “cellular material” is substantially intracellular material that would not have otherwise been released without lysis. The term “cellular material” also includes material that would otherwise have remained membrane bound.

“Cellular material” includes within its scope genetic material (all forms thereof, including nucleic acids, polynucleotides and more specifically genomes, genes, gene transcripts, gene products and RNA), proteinaceous material (all forms thereof, including polypeptides, proteins, peptides and amino acids), lipid materials (all forms thereof, including fats and lipids), and carbohydrate materials (all forms thereof).

“Cellular material” includes within its scope all of the components or structures in cellular systems.

A downstream application includes any and all molecular-based methods and procedures. Such methods and procedures can be quantitative, qualitative, for selective characterisation, modification, isolation or amplification et cetera. A downstream application can be a screening test or diagnostic test, to identify or confirm any change/s to the cellular material of the oocyte, blastocyte/blastocyst, ovum, embryonic cell, embryo or spermatozoa.

A downstream application can comprise subjecting the cellular material to the action of at least one exogenously added enzyme, such as a protein-based enzyme or RNA based enzyme.

A downstream application can be for, for example, the study of genes (genomics and epigenomics), transcripts (transcriptomics), proteins (proteomics), metabolites (metabolomics), lipids (lipidomics) or interactions (interactomics).

Potential downstream applications for the cellular material are described in Wang and Bodovitz, Trends Biotechnol. 2010 June; 28 (6): 281-290, the entire contents of which are incorporated herein by way of cross-reference. Other potential downstream applications are described elsewhere in this specification.

The term “compatible with a downstream application” preferably means that the downstream application can be carried out in the lysis solution itself, or with minimal modification of the lysis solution. The term preferably means that the lysis solution is not inhibitory to the function of one or more enzymes used in the downstream application.

In another aspect of the invention, the inventors have developed a lysis solution for selectively lysing an oocyte, blastocyte/blastocyst, ovum, embryonic cell or embryo in the presence of spermatozoa and for selectively releasing genetic material from only the lysed oocyte, blastocyst, ovum, embryonic cell or embryo such that the released genetic material within the lysis buffer is capable of being selectively replicated using a polymerase enzyme.

In yet another aspect of the invention, the inventors have developed a method of selectively lysing an oocyte, blastocyte/blastocyst, ovum, embryonic cell or embryo in the presence of spermatozoa such that genetic material from a lysed oocyte, blastocyst, ovum, embryonic cell or embryo is capable of being selectively replicated using a polymerase enzyme within the lysis solution.

In another aspect of the invention, the inventors have developed a method that requires little manipulation and washing of an oocyte, blastocyte/blastocyst, ovum, embryonic cell or embryo and is amenable to replication by a polymerase enzyme. This is accomplished by differentially lysing the oocyte, blastocyst, ovum, embryonic cell or embryo in the presence of spermatozoa, whilst keeping spermatozoa intact. DNA from a single lysed oocyte, blastocyst, ovum, embryonic cell or embryo can then be replicated in a downstream application, for example, to yield ample whole genomic DNA for a variety of further downstream applications. In some embodiments the method is robust, consistent, sensitive and non-inhibitory to downstream applications such as whole genome amplification, qPCR, micro array or sequencing.

According to a 4^{th} aspect of the present invention, there is provided a method of selectively lysing an oocyte, blastocyte/blastocyst, ovum, embryonic cell or embryo in the presence of spermatozoa such that genetic material from a lysed oocyte, blastocyst, ovum, embryonic cell or embryo is capable of being selectively replicated using a polymerase enzyme, said method comprising the steps of:

(1) subjecting the oocyte, blastocyst, ovum, embryonic cell or embryo to a lysis solution such that the oocyte, blastocyst, ovum, embryonic cell or embryo is lysed and genetic material is released from the oocyte, blastocyst, ovum, embryonic cell or embryo, but such that the spermatozoa is not lysed; and

(2) selectively replicating the released genetic material within the lysis buffer using a polymerase enzyme.

According to a 5^{th} aspect of the present invention, there is provided released genetic material when produced by the method of the 1^{st} aspect.

According to a 6^{th} aspect of the present invention, there is provided a lysis solution for selectively lysing an oocyte, blastocyte/blastocyst, ovum, embryonic cell or embryo in the presence of spermatozoa that is not lysed by the lysis solution, and for selectively releasing genetic material from the lysed oocyte, blastocyst, ovum, embryonic cell or embryo such that the released genetic material within the lysis buffer is capable of being selectively replicated using a polymerase enzyme.

The oocyte, blastocyte/blastocyst, ovum, embryonic cell, embryo or spermatozoa may have previously been (knowingly or unknowingly) genetically manipulated or mutagenised or not.

The method can be used as a downstream application screening test or diagnostic test, to identify or confirm any change/s to the genetic material of the oocyte, blastocyte/blastocyst, ovum, embryonic cell, embryo or spermatozoa.

Potential uses include determining the ability of test regimes or treatments to change the genotype of transgenic animals or fertilized ova resulting from in vitro fertilisation or sexing of embryos. Genes that can be detected include SRY, chromosome I, 12S, GAPDH, ACTB, chromosome Y, or any desirable gene product prior to advancing treatments to expensive animal trails. Downstream applications such as whole genome analysis (eg microarray) and population studies can be conducted using this method.

Change or absence of gene products can be brought about by treatment of spermatozoa and/or oocytes by: CRISPR (Clustered Regulatory Interspaced Short Palindromic Repeats gene editing); SMGT (Sperm-Mediated Gene Transfer) (Rodrigues 2013); TALEN (Transcription Activator-Like Effector Nucleases); antibodies blocking docking sites on spermatozoa; antibodies blocking docking sites on oocytes; any chemical such as sunscreen, detergent, talc etc; and chemicals that may have a mutagenic effect.

Regarding the downstream application, any suitable type of polymerase enzyme can be used. Preferably a DNA polymerase enzyme is used although an RNA polymerase enzyme could be used in some instances. Examples of suitable DNA polymerase enzymes include the following: ϕ29 DNA polymerase, T7 DNA polymerase, Thermostable Taq Polymerase (from Thermis aquaticus) (native or recombinant), Pfu DNA polymerase (from Pyrococcus furiosus) for high fidelity, Hot-start DNA polymerases to suppress nonspecific product amplification when more than one set of primers are used, High-fidelity polymerases (Hi-Fi) with proofreading activity. Primers can include Poly T (if eukaryotic RNA is targeted), random hexamers, random pentamers or random octamers. Primers can also be chosen to have specific attributes such as exonuclease-resistance or endonuclease resistance.

The actual polymerase enzyme used will depend on the reason for which the genetic material is to be replicated—ie. the further downstream application. For example, the further downstream application can be next generation sequencing, microarray, single, duplex or multiplex PCR, or real-time single, duplex or multiplex PCR. The genetic material can be pre-amplified to yield ample whole genomic DNA for a variety of further downstream applications.

The method can be used to lyse and amplify DNA for PCR or Real Time PCR (qPCR) from a single unfertilised or fertilised ovum to embryos that have developed into 2, 4, 8, 16 or more cells. Preferably the method is robust, consistent, sensitive, nor inhibitory to downstream applications such as multiple displacement amplification (MDA), whole genome amplification (WGA), qPCR, micro array or sequencing.

The genetic material that is released from the lysed oocyte, blastocyst/blastocyte, ovum, embryonic cell or embryo is preferably genomic deoxynucleic acid (DNA) but other types of polynucleotides/nucleic acids may be released as well, e.g. ribonucleic acid (RNA).

The method can include the step of genetically manipulating the oocyte, blastocyst, ovum, embryonic cell, embryo or sperm prior to subjecting it to the lysis solution. The method can include the step of exposing the spermatozoa/semen to a chemical such as a mutagen prior to fertilisation. The method can include the step of subjecting the oocyte, blastocyst, ovum, embryonic cell, embryo or spermatozoa to an IVF technique, which potentially could lead to modification of the genetic material.

The oocyte, blastocyst, ovum, embryonic cell, embryo or spermatozoa can be manipulated, for example, using gene editing techniques Clustered Regulatory Interspaced Short Palindromic Repeats (CRISPR) and Transcription Activator-Like Effector Nucleases (TALEN). For example, the spermatozoa/semen can be exposed to chemicals, such as known or suspected mutagens. Sex selection treatments of semen as well as sex selection treatments of embryos can potentially lead to genetic modification of the fertilised oocyte, blastocyst, ovum or embryo.

In some embodiments the method can be used for high-throughput in vitro fertilisation (HT-IVF), as outlined below:
Treat oocytes from pig (or other) ovaries to enhance maturation in a newly developed medium (as described in the first section of this specification to do with IVP).
Fertilise with fresh, extended pig (or other) semen.
Grow out to 8-16 cells→65% of oocytes mature and grow in new procedure (industry standard about 40%).
Lyse oocytes and residual semen differently with a novel lysis solution, allowing oocyte DNA to be extracted leaving spermatozoa intact.
Amplify oocytes DNA using qPCR by REPLI g kit SC Polymerase (Qiagen).
Dilute DNA to detect a range of genotypic traits by real time PCR (qPCR)
Change or absence of gene products can be brought about by treatment of spermatozoa and/or oocytes by:
   CRISPR ((Clustered Regulatory Interspaced Short Palindromic Repeats gene editing)
   SMGT (Sperm-Mediated Gene Transfer) (Rodrigues 2013)
   TALEN (Transcription Activator-Like Effector Nucleases)
   antibodies blocking docking sites on spermatozoa
   antibodies blocking docking sites on oocytes
   any chemical such as sunscreen, detergent, talc, etc
   chemicals that may have a mutagenic effect
Uses:
   Determine the ability of test regimes or treatments to change genotype
   Transgenic animals
   In vitro fertilisation
   Sexing of embryos

Genes or gene regions detected: SRY, Chromosome I, 12S, GAPDH, ACTB, chromosome Y, any desirable gene product, prior to advancing treatments to expensive animal trails. Whole genome analysis (eg microarray) and population studies can be conducted using this method.

The oocyte, blastocyte/blastocyst, ovum, embryonic cell or embryo can be sourced from a human or any suitable type of animal. The animal can be a mammal. The animal can be a farm animal such as a pig, cow, horse, sheep or goat. The animal can be a companion animal such as a dog or cat. The animal can be a laboratory animal such as a rabbit, mouse or rat.

Any suitable type of lysis solution can be used. A suitable lysis solution can have one or more of the following types of ingredients: salt; lytic enzyme; destabiliser; metal chelator; reducing agent; detergent; buffer; and, pH adjuster. General properties of a suitable lysis solution are shown in Table 1B below.

TABLE 1B
Properties of a suitable lysis solution.

| | | Specific |
|---|---|---|
| | | examples and |
| | Concentration range | concentration |
| Ingredient type | (%w/v or mM range) | ranges |
| Salt | 10-200mM NaCI | 50 mM NaCl, |
| Other ingredients | 0.1-1 mMEDTA, | Sucrose |
| Sugars, EDTA, | 100 pg/ml-0.5 mg/ml | 100 mM |
| SDS, Proteinase K, | Proteinase K, sucrose 5- | |
| Dithiothreitol (DTT), | 250 mM, DTT 10-40 | |
| De-oxycholate (bile | mM, bile salts 1.5-5 | |
| salts), trypsinase, NaOH, | mM, 5 μg/ml, NaOH | |
| KOH, lysozyme, NaOH, | 5-50 mM, KOH 5- | |
| KOH, Bovine serum | 50 mM, lysozyme 1-3 | |
| albumin (BSA) | mg/ml, BSA (0.1-5%) | |
| Detergent | 0.001-10% NP40, Nonionic | 0.12% |
| | 0.001-10% Tween 20, | TRITON |
| | Nonionic | X-100 |
| | 0.001-10% SDS, anionic | |
| | CHAPS Zwitterionic | |
| | CTAB 0.001-0.01% | |
| | Zwitterionic | |
| | TRITON X-100 0.001- | |
| | 10% Nonionic | |
| Buffer | 1-250 mM Tris-HCL | 10 mM |
| | 10 mM citrate buffer | Tris-HCL |
| | 50 mM HEPES, RIPA | |
| pH | 5.4-8.3 | 7.5 |

Typically, the lysis solution will have the properties shown in Table 2B below.

TABLE 2B
Properties of a preferred lysis solution.

| Ingredient | Concentration range | Specific examples and |
|---|---|---|
| type | (% w/v or mM range) | concentration ranges |
| Salts | 50-150 mM | 50 mM NaCl |
| | NaCl, KCl, (NaH₄)₂SO₄ | |
| Lytic | Proteinase K 100 | Hyaluronidase 10 μg/ml |
| Enzymes | pg/ml-20 mg/ml. | Proteinase K 20 mg/ml. |
| | lysozyme 1-3 mg/ml, | Trypsin-EDTA 0.0005% |
| | Hyaluronidase 100 | |
| | pg/ml-0.5 mg/ml. | |
| | Trypsin-EDTA | |
| | 0.00005-50% | |
| Destabilisers | 1.5-25% sugar | Sucrose 100 mM |
| | 0.1-5% BSA | |
| Metal | 0.1-2 mM EDTA | |
| Chelators | 0.1-2 mM EGTA | |
| Reducing | 1-10 mM for all | |
| Agents | Dithiothreitol (DTT) | |
| | DTE | |
| | 2-Mercaptoethanol | |
| Detergent | 0.2-2% NP40, Nonionic | Triton X-100 0.12% |
| | 2-10% Tween 20, | |
| | Nonionic | |
| | 0.1-2% SDS, anionic | |
| | CHAPS Zwitterionic | |
| | CTAB 0.001-0.01% | |
| | Zwitterionic | |
| | TRITON X-100 0.1-5%, | |
| | Nonionic | |
| Buffer | 10-150 mM Tris-HCL | 100 mM Tris-HCL |
| | 10 mM citrate buffer | |
| | 50 mM HEPES, RIPA | |
| pH | 5.4-8.3 | 7.5 |

See the general reference: ALCARAZ, C., DE DIEGO, M., PASTOR, M. J. & ESCRIBANO, J. M. 1990. Comparison of a radioimmunoprecipitation assay to immunoblotting and ELISA for detection of antibody to African swine fever virus. J Vet Diagn Invest, 2, 191-6.

The oocyte, blastocyte/blastocyst, ovum, embryonic cell or embryo can be suspended in any suitable type of solution prior to the lysis solution. In some embodiments the oocyte, blastocyst, ovum, embryonic cell or embryo can be suspended in the culture medium such as M-199, water, PBS or as otherwise described in the section 1 of this specification entitled “Reproduction Methodologies”. It can be suspended in any suitable volume but that volume will typically be in the microlitre range.

In step (1) or other like method step as described herein, lysis can occur for any suitable period time and at any suitable temperature. For example, a suitable period of time can be anywhere between 5 seconds and 5 hours. For example, a suitable temperature can be anywhere between 14° and 100° C., but is preferably about 38° C. for a period of 10 minutes, 55° C. for a period of 10 minutes, and 95° C. for a period of 5 minutes.

The method can include the step of heating the mixture of step (1) or other like method step as described herein to an elevated temperature so as to selectively activate and inactivate enzymes present in the mixture. A suitable temperature can be anywhere between 14° and 100° C. Preferably the temperature is about 85° C. Preferably the mixture of step (1) or other like method step as described herein is heated at about 38° C. for a period of 10 minutes, 55° C. for a period of 10 minutes and 95° C. for a period of 5 minutes.

In step (1) or other like method step as described herein, after lysis and inactivation, the mixture can be cooled on ice until step (2) or other like method step as described herein is undertaken.

In step (2) or other like method step as described herein the conditions will depend on the polymerase technique or techniques of interest—ie. the further downstream application or applications. For example, the downstream application can be next generation sequencing, microarray, single, duplex or multiplex PCR, or real-time single, duplex or multiplex PCR. The genetic material can be pre-amplified to yield ample whole genomic DNA for a variety of downstream applications.

Step (2) or other like method step as described herein can be used to lyse and amplify DNA for PCR or Real Time PCR (qPCR) from a single unfertilised or fertilised ovum to embryos that have developed into 2, 4, 8, 16 or more cells.

General techniques and methodologies for oocyte handling, sperm handling, in vitro fertilization, embryo handling can be found in the following references, each of which is incorporated herein in its entirety by way of cross-reference: Fléchon et al., 2003, Bahnak et al., 1988, Mao et al., 2013, Garcia-Vazquez et al., 2016, Garcia-Vazquez et al., 2015, Hennekens et al., 2013, Rodriguez-Martinez, 2013, Romar et al., 2016, Broekhuijse et al., 2012, Lopez Rodriguez et al., 2017, Bredbacka et al., 1995, and Wieczorek et al., 2015.

General techniques and methodologies for suitable polymerase techniques of interest can be found in the following references, each of which is incorporated herein in its entirety by way of cross-reference: Jiang et al., 2005, Chen and Kuo, 2011, Martín et al., 2009, Khamlor et al., 2014, Li et al., 2011, Bredbacka et al., 1995, Hirayama et al., 2004, Kirkpatrick and Monson, 1993, Pomp et al., 1995, and Torner et al., 2013.

Any of the features described herein can be combined in any combination with any one or more of the other features described herein within the scope of the invention.

The reference to any prior art in this specification is not, and should not be taken as an acknowledgement or any form of suggestion that the prior art forms part of the common general knowledge.

### BRIEF DESCRIPTION OF DRAWINGS 2

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

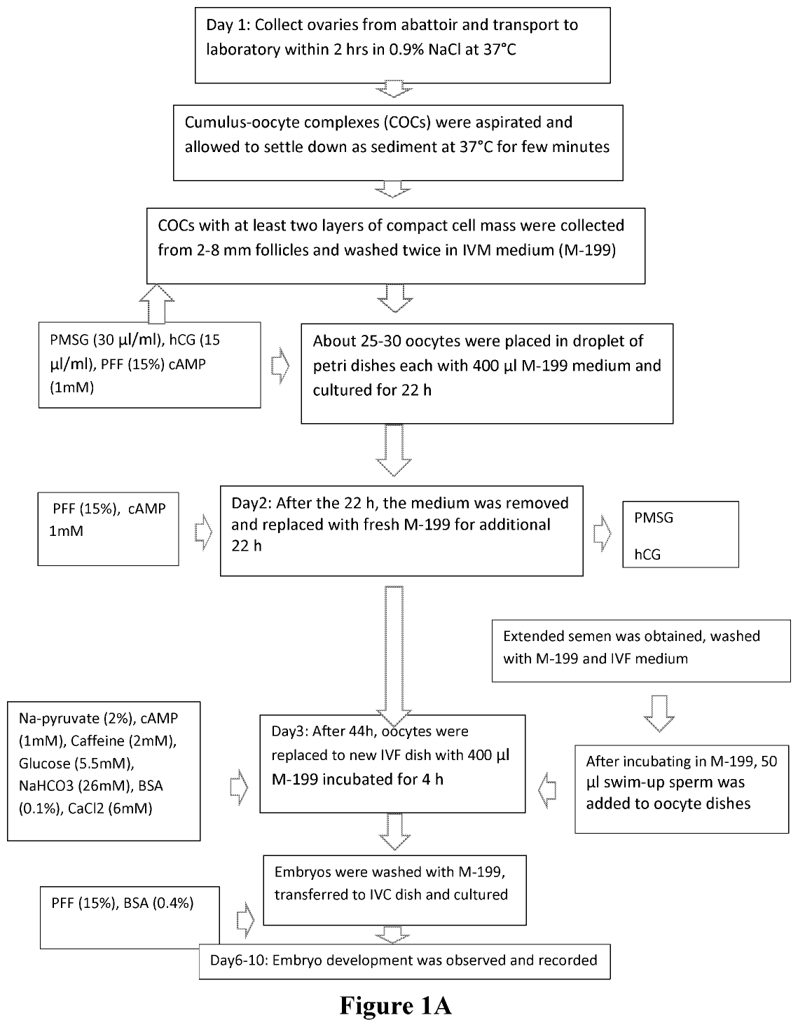

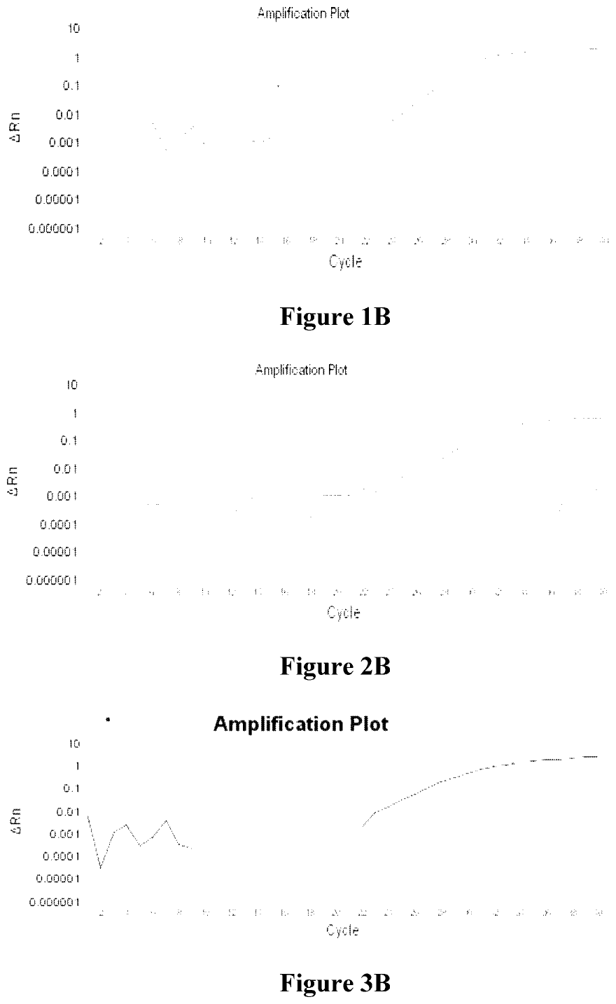

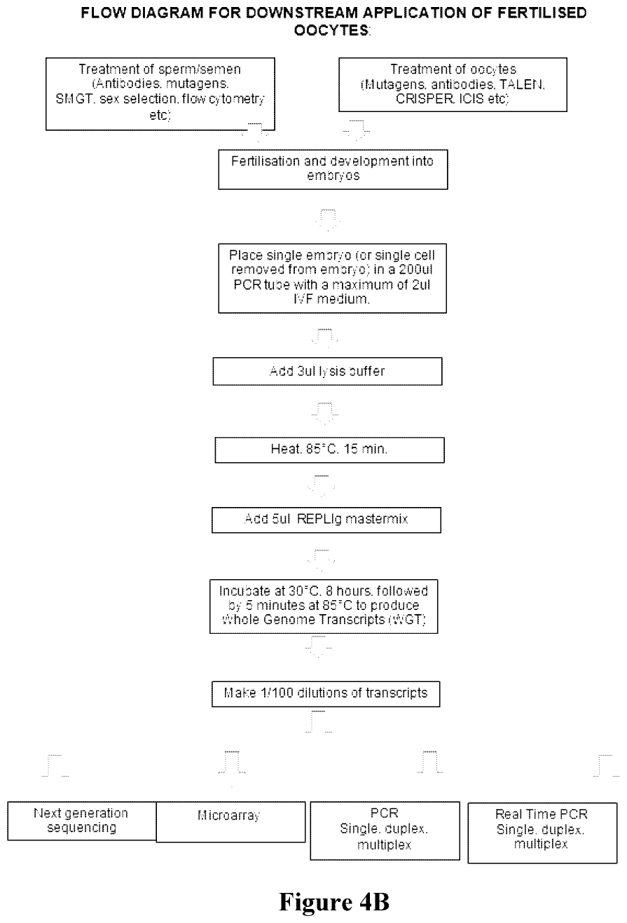

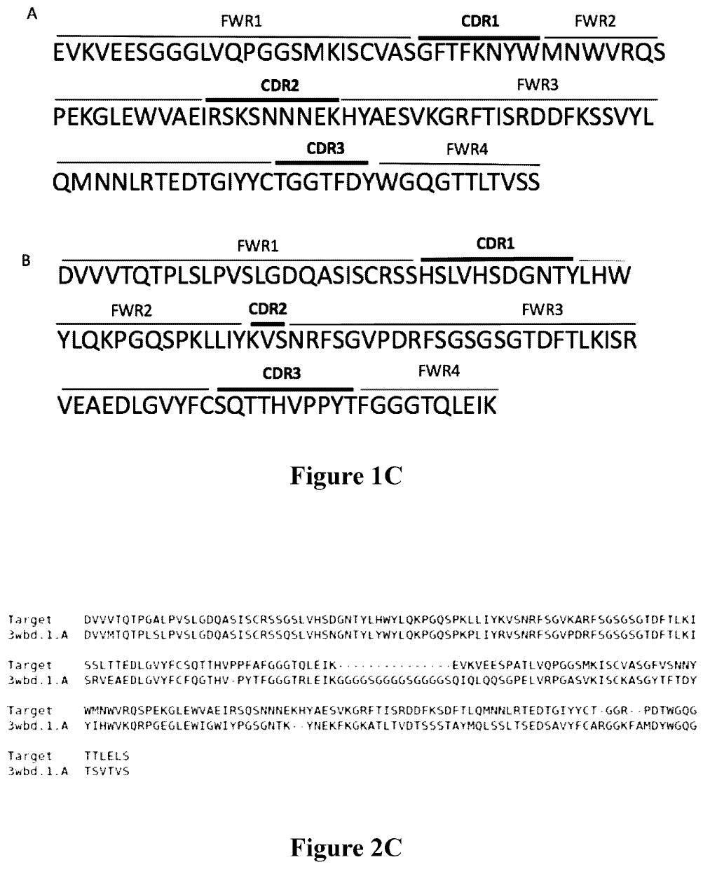

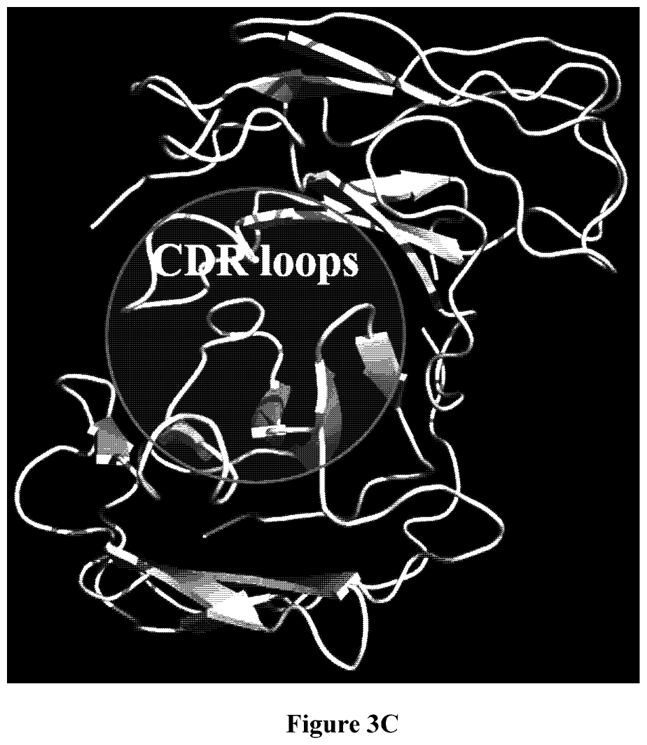

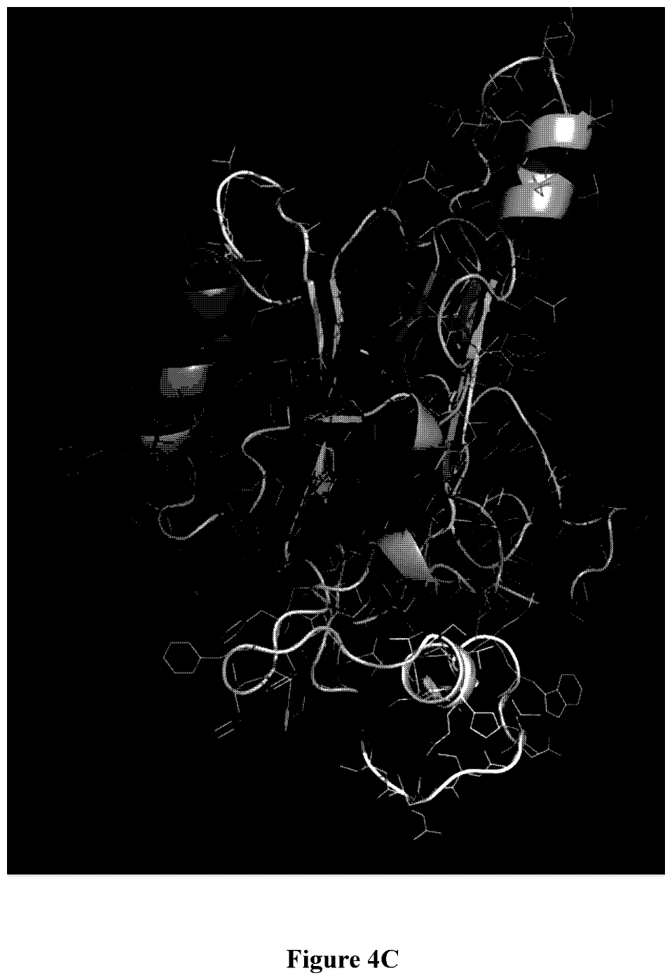

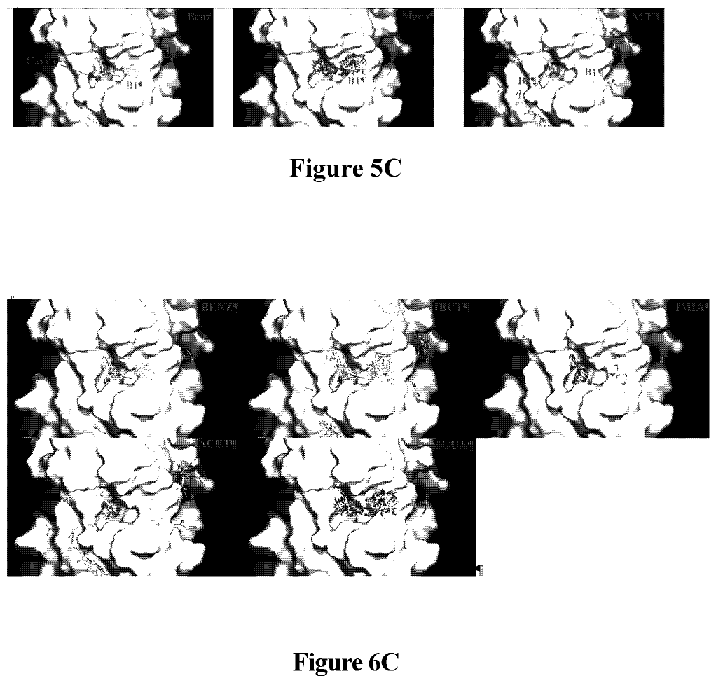

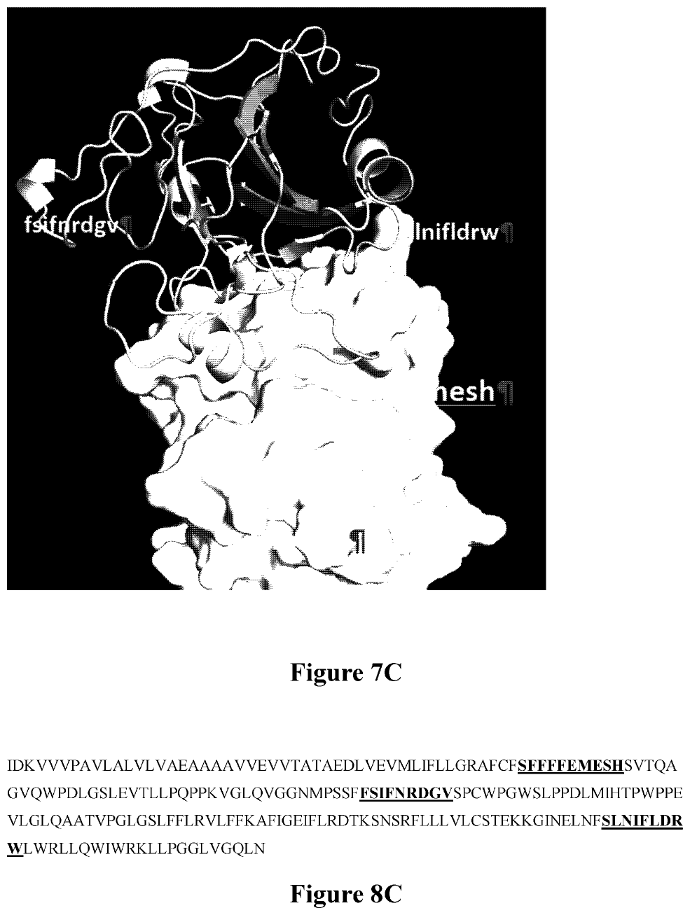

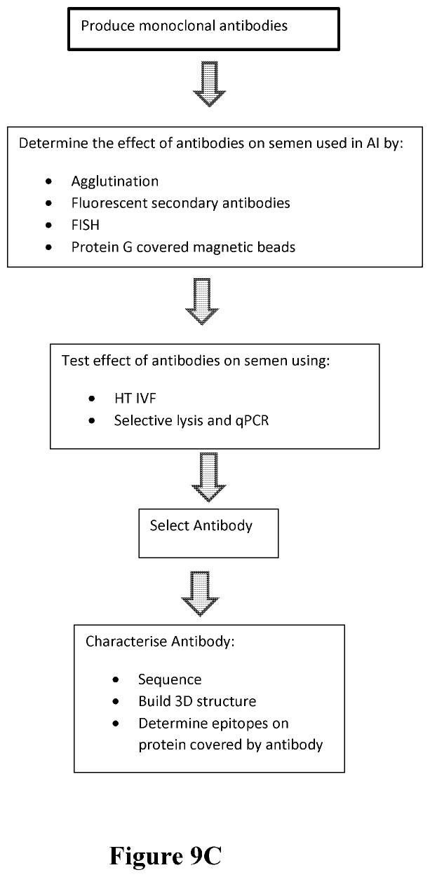

FIG. 1B. Amplification plot for Chromosome Y, indicating the Threshold and wells with Chromosome Y amplicons.

FIG. 2B. Amplification plot for Chromosome 12S, indicating the Threshold and wells with Chromosome 12S amplicons.

FIG. 3B. Amplification plot for Chromosome 12S and Chr Y combined, indicating the Threshold and wells with Chromosome 12S and Chr Y amplicons

### DETAILED DESCRIPTION OF EMBODIMENTS 2

### EXAMPLE 1

### Differential Lysis of an Embryo or Ovum in the Presence of Spermatozoa or Semen, for Polymerase Enzyme Amplification

This Example describes the use of a novel lysis buffer/solution for differential lysis of an embryo or ovum in the presence of spermatozoa or semen, for PCR amplification. DNA from a single lysed embryo or ovum can then be pre-amplified by PCR to yield ample whole genomic DNA for a variety of downstream applications.

The high throughput method described here is a rapid, 25 minute one-tube procedure delicate enough to lyse oocytes selectively in the presence of spermatozoa and is at the same time sensitive enough to lyse and pre-amplify DNA from single, unfertilised ova and embryos that have developed into 2, 4, 8, 16 or more cells. Furthermore, no substances are introduced that could be inhibitory to the pre-amplification or downstream applications. The method is also compatible with the Single-cell REPLIg kit as well as further qPCR applications.

Materials and Methods:

Pre-Amplification

Single, fertilised embryos (two or more cell stage) plus medium in 2 μl volumes were transferred to 200 μl PCR tubes. Lysis solution/buffer (4 μl) as shown in Table 2B, consisting of 50 mM NaCl, 10 μg/ml hyaluronidase, 0.0005% Trypsin-EDTA, 20 mg/ml Proteinase K, 100 mM sucrose, 0.12% Triton X-100 in 100 mM Tris-HCL pH 7.5 was added, heated for 38° C. for a period of 10 minutes, 55° C. for a period of 10 minutes and 95° C. for a period of 5 minutes, and then cooled on ice. REPLIg master mix (7.75 μl) consisting of 7.25 μl of REPLIg sc reaction buffer and 0.5 μl REPLIg sc Polymerase were added directly to the lysed cells in the same tube. Tubes were vortexed well, spun down to collect all material at the bottom of the tube and incubated at 30° C. for 8 hours, followed by 65° C. for 3 minutes to denature the sc Polymerase. The controls for each run included unfertilised ova, a no template control, 1 μl of semen and a no reagent control.

The pre-amplified whole genome DNA can at this point be used for a variety of applications, such as PCR, qPCR, or micro array.

The lysed oocytes and genomic material can also be used directly as template for PCR, real-time PCR. Nine (9) μl of Mastermix, (consisting of 2×TaqMan Gene Expression Master Mix (Life Technologies), 0.4 μM each of Chromosome Y Forward and reverse primers, 0.25 μM Chromosome Y FAM probe, 0.4 μM each of Chromosome 12S or Chromosome 1 forward and reverse primer and 0.25 μM Chromosome 1 or Chromosome 12S (Martin 2009) VIC probe.) was added to the total volume of lysate. Chromosome 12S was used as an internal control. The assay was run using the following conditions: A holding period of 50° C. for 2:00 min, followed by 95° C. for 10:00 min and then 40 cycles of 95° C. for 15 seconds, and 60° C. for 60 sec. Apart from the control wells for the pre-amplification (unfertilised ova, a no template control (NTC), 1 μl of semen and a no reagent control), a further genomic DNA (gDNA) and NTC were added. See Table 3B.

Real Time PCR (qPCR)

The scDNA from the pre-amplification was diluted 1/100 and 1 μl was used in a 10 μl PCR reaction as template. Master mix, consisting of 2×TaqMan Gene Expression Master Mix (Life Technologies), 0.4 μM each of Chromosome Y Forward and reverse primers, 0.25 μM Chromosome Y FAM probe, 0.4 μM each of Chromosome 12S or Chromosome 1 forward and reverse primer and 0.25 μM Chromosome 1 or Chromosome 12S (Martin 2009) VIC probe. Chromosome 12S was used as an internal control. The assay was run using the following conditions: A holding period of 50° C. for 2:00 min, followed by 95° C. for 10:00 min and then 40 cycles of 95° C. for 15 seconds, and 60° C. for 60 sec. Apart from the control wells for the pre-amplification (unfertilised ova, a no template control (NTC), 1 μl of semen and a no reagent control), a further genomic DNA (gDNA) and NTC were added. See Table 3B.

TABLE 3B
PCR primers/probes

| | | | GC | Amp- |
|---|---|---|---|---|
| | Sequence | Tm | % | licon |
| 12S | | | | |
| Forward | CACCCTCCTCAAGCATGTAGTAATAA | 59 | 42 | 86 bp |
| Primer | | | | |
| (SEQ ID | | | | |
| NO. 366) | | | | |
| Reverse | GCTTACCTTGTTACGACTTGTCTCTTC | 59 | 44 | |
| Primer | | | | |
| (SEQ ID | | | | |
| NO. 367) | | | | |
| Probe | CTATATTCAATTACACAACCATG | 69 | 30 | |
| (SEQ ID | | | | |
| NO. 368) | | | | |
| Chromo- | | | | |
| some 1 | | | | |
| Forward | TGCCACACAAGGCATATTCTG | 58 | 48 | 64 bp |
| Primer 1 | | | | |
| (SEQ ID | | | | |
| NO. 369) | | | | |
| Reverse | CAACTCCAAACGTGCTCTACTTCA | 59 | 46 | |
| Primer 1 | | | | |
| (SEQ ID | | | | |
| NO. 370) | | | | |
| Probe 1 | ATCCGCCTCCTCC | 68 | 69 | |
| Chromo- | | | | |
| some-Y | | | | |
| Forward | AATCCACCATACCTCATGGACC | 70 | 50 | |
| Primer 2 | | | | |
| (SEQ ID | | | | |
| NO. 371) | | | | |
| Reverse | GCAGGAGGATACAGGAGAAA | | | |
| Primer 2 | | | | |
| (SEQ ID | | | | |
| NO. 372) | | | | |
| Probe 2 | ACTTTCTTGGGAGAGCAC | | | |
| (SEQ ID | | | | |
| NO. 373) | | | | |

Results:

For assay results to be accepted as correct, unfertilised ova should have a positive signal for Chr 1 or 12S, with an absent Chr Y signal. Negative reactions (no Chr 1, 12S or Chr Y signal) for semen, no template and no reagent controls are expected. Genomic DNA should be positive for both Chr Y and Chr 1 or 12S. Ct values <35.5 were considered positive and if the Ct value was >35.5 the result was disregarded as a negative. No amplicon in tubes, or an absent 12S and present Chr Y indicated amplification failure.

FIG. 1B shows the amplification plot for Chromosome Y, indicating the Threshold and wells with Chromosome Y amplicons. FIG. 2B shows amplification plot for 12S, indicating the Threshold and wells positive for Chromosome 12S. FIG. 3B shows the amplification plot for Chromosome 12S and Chr Y combined, indicating the Threshold and wells with Chromosome 12S and Chr Y amplicons.

Data from oocytes fertilised with treated semen appear in Table 4B. The target genes were 12S and Chr Y, effectively sexing the fertilised oocytes. Controls (n=8) had 58% females vs 42% females; oocytes fertilised with semen from the top of the tubes (n=16) had 84% females and 16% males. The oocytes fertilised with semen from the bottom of the tubes (n=8), had 75% females and 25% males.

TABLE 4B
Example of data from oocytes fertilised with treated semen.
Two genes, 12S, and internal control and Chr Y were
detected. The combination of the two results was used
to determine the sex of the embryos.

| | Sample | Target | | |
|---|---|---|---|---|
| Well | Name | Name | 12S | ChrY |
| A1 | gDNA | 12S | 1 | 1 |
| A1 | gDNA | Chr Y | | |
| B1 | NTC | 12S | 0 | 0 |
| B1 | NTC | Chr Y | | |
| C1 | Semen | 12S | 0 | 0 |
| C1 | Semen | Chr Y | | |
| D1 | UFO | 12S | 1 | 0 |
| D1 | UFO | Chr Y | | |
| E1 | NTCrep | 12S | 0 | 0 |
| E1 | NTCrep | Chr Y | | |
| F1 | C1 | 12S | 1 | 0 |
| F1 | C1 | Chr Y | | |
| G1 | C2 | 12S | 1 | 1 |
| G1 | C2 | Chr Y | | |
| H1 | C3 | 12S | 1 | 1 |
| H1 | C3 | Chr Y | | |
| A2 | C4 | 12S | 1 | 1 |
| A2 | C4 | Chr Y | | |
| B2 | C5 | 12S | 1 | 1 |
| B2 | C5 | Chr Y | | |
| C2 | C6 | 12S | 1 | 0 |
| C2 | C6 | Chr Y | | |
| D2 | C7 | 12S | 1 | 0 |
| D2 | C7 | Chr Y | | |
| E2 | C8 | 12S | 0 | 1 |
| E2 | C8 | Chr Y | | |
| | | | 7 | 5 |
| | | | % females | % males |
| | | Control | 58.33333 | 41.66667 |
| F2 | T1 | 12S | 1 | 0 |
| F2 | T1 | Chr Y | | |
| G2 | T2 | 12S | 1 | 0 |
| G2 | T2 | Chr Y | | |
| H2 | T3 | 12S | 1 | 0 |
| H2 | T3 | Chr Y | | |
| A3 | T4 | 12S | 1 | 0 |
| A3 | T4 | Chr Y | | |
| B3 | T5 | 12S | 1 | 0 |
| B3 | T5 | Chr Y | | |
| C3 | T6 | 12S | 1 | 1 |
| C3 | T6 | Chr Y | | |
| D3 | T7 | 12S | 1 | 0 |
| D3 | T7 | Chr Y | | |
| E3 | T8 | 12S | 1 | 0 |
| E3 | T8 | Chr Y | | |
| F3 | T9 | 12S | 1 | 1 |
| F3 | T9 | Chr Y | | |
| G3 | T10 | 12S | 1 | 0 |
| G3 | T10 | Chr Y | | |
| H3 | T11 | 12S | 1 | 0 |
| H3 | T11 | Chr Y | | |
| A4 | T12 | 12S | 1 | 0 |
| A4 | T12 | Chr Y | | |
| B4 | T13 | 12S | 1 | 0 |
| B4 | T13 | Chr Y | | |
| C4 | T14 | 12S | 1 | 1 |
| C4 | T14 | Chr Y | | |
| D4 | T15 | 12S | 1 | 0 |
| D4 | T15 | Chr Y | | |
| E4 | T16 | 12S | 1 | 0 |
| E4 | T16 | Chr Y | | |
| | | | 16 | 3 |
| | | | % females | % males |
| | | Top | 84.21053 | 15.78947 |
| F4 | B1 | 12S | 1 | 1 |
| F4 | B1 | Chr Y | | |
| G4 | B2 | 12S | 1 | 1 |
| G4 | B2 | Chr Y | | |
| H4 | B3 | 12S | 1 | 0 |
| H4 | B3 | Chr Y | | |
| A5 | B4 | 12S | 0 | 0 |
| A5 | B4 | Chr Y | | |
| B5 | B5 | 12S | 1 | 0 |
| B5 | B5 | Chr Y | | |
| C5 | B6 | 12S | 0 | 0 |
| C5 | B6 | Chr Y | | |
| D5 | B7 | 12S | 1 | 0 |
| D5 | B7 | Chr Y | | |
| E5 | B8 | 12S | 1 | 0 |
| E5 | B8 | Chr Y | | |
| F5 | Diluent | 12S | 0 | 0 |
| F5 | Diluent | Chr Y | | |
| | | | 6 | 2 |
| | | | % females | % males |
| | | Bottom | 75 | 25 |

Discussion

Detection of Chr 1 or 12S indicates signal from the unfertilised ova alone. Negative reactions (no Chr 1, 12S or Chr Y signal) for semen indicates that there is no lysis from the semen sample and therefore no gene contribution is expected from any possible residual semen in the fertilised ova wells. Chr 1 and 12S were internal controls and the absence of this amplicon, even in the presence of a Chr Y amplicon, was disregarded as amplification failure.

Genomic DNA was positive for both 12S and Chr Y, while NTC, semen and NTC from REPLIg had no DNA detected, evident by the absence of any amplicons. The unfertilised oocyte control (UFO) was positive for 12S, as expected from porcine genomic material, but no Chr Y that could have been contributed from accidentally lysed semen was detected. This result was interpreted as “female”.

This High Throughput In Vitro Fertilisation (HT IVF) method can have various applications. See FIG. 4B. Ova can be fertilised by semen that is treated by SMGT (Sperm-Mediated Gene Transfer) (Rodrigues 2013), flow cytometry, addition of antibodies or any chemical such as sunscreen, detergent, talc, or suspected or known mutagens. The effect of the treatment on semen that can be transferred when fertilisation takes place can be tested on a single gene, such as Chr Y, in this example. As the method uses whole genome amplification, the effect of semen treatment can be determined on the whole genome of the offspring. Furthermore, as multiple oocytes can undergo the same treatment, the effect on whole populations of offspring can be determined.

For the same effect, untreated semen can be used on ova that have been genetically manipulated by techniques such as CRISPR (Clustered Regulatory Interspaced Short Palindromic Repeats (gene editing) or TALEN (Transcription Activator-Like Effector Nucleases). The effects of addition to ova of antibodies or a chemical such as sunscreen, detergent, talc, or suspected or known mutagens can be tested on singular and populations of embryos.

### REFERENCES 2

Patent US 20160222375 A1 Method, apparatus and kit for human identification using polymer filter means for separation of sperm cells from biological samples that include other cell types.

Patent EP 2 284 256 A2 Sperm cell insemination samples having selectably controlled sperm cell fertility characteristics produced through entrainment in a fluid stream having correspondingly selectably adjustable flow characteristics and methods of assessing comparative of sperm cell insemination sample fertility.

Patent U.S. Pat. No. 6,548,741 B2 DEVELOPMENTAL COMPETENCE FOR ASSISTED REPRODUCTION AND NUCLEAR TRANSFER IN PIGS

Patent US20150232917A1 Differential lysis with aid of Alkali and pressure

Gill, Peter, Jeffreys, Alec J and Werrett, David J. (1985) Forensic application of DNA ‘fingerprints’. Nature Volume: 318 Issue: 6046 Pages: 577-579

Mao, Shihong, Goodrich, Robert J., Hauser, Russ, Schrader, Steven M., Chen, Zhen and Krawetz, Stephen A. (2013) Evaluation of the effectiveness of semen storage and sperm purification methods for spermatozoa transcript profiling. Systems biology in reproductive medicine. Volume: 59 Issue: 5 Pages: 287-295

Martín, Irene, García, Teresa, Fajardo, Violeta, Rojas, María, Pegels, Nicolette, Hernández, Pablo E, González, Isabel and Martín, Rosario. (2009) SYBR-Green real-time PCR approach for the detection and quantification of pig DNA in feedstuffs. Meat Science Volume: 82 Issue: 2 Pages: 252-259

Norris, Jessica V., Manning, Kate, Linke, Sarah J., Ferrance, Jerome P. and Landers, James P. Year: (2007) Expedited, Chemically Enhanced Sperm Cell Recovery from Cotton Swabs for Rape Kit Analysis. Journal of Forensic Sciences Volume: 52 Issue: 4 Pages: 800-805

Pomp, D, Good, B A, Geisert, R D, Corbin, C J and Conley, A J. (1995) Sex identification in mammals with polymerase chain reaction and its use to examine sex effects on diameter of day-10 or -11 pig embryos. Journal of animal science. Volume: 73 Issue: 5 Pages: 1408-1415

Rodriguez-Martinez, Heriberto. (2013) Sperm biotechnologies in domestic species: state of the art. Animal Reproduction Volume: 10 Pages: 268-276

Rossen, Lone, Nørskov, Pernille, Holmstrøm, Kim and Rasmussen, Ole F. (1992) Inhibition of PCR by components of food samples, microbial diagnostic assays and DNA-extraction solutions. International Journal of Food Microbiology. Volume: 17 Issue: 1 Pages: 37-45

Torner, Eva, Bussalleu, Eva, Briz, M. Dolors, Gutiérrez-Adán, Alfonso and Bonet, Sergi. (2013) Sex determination of porcine embryos using a new developed duplex polymerase chain reaction procedure based on the amplification of repetitive sequences. Reproduction, Fertility and Development. Volume: 25 Issue: 2 Pages: 417-425

Yoshida, Kanako, Sekiguchi, Kazumasa, Mizuno, Natsuko, Kasai, Kentaro, Sakai, Ikuko, Sato, Hajime and Seta, Sueshige (1995); The modified method of two-step differential extraction of sperm and vaginal epithelial cell DNA from vaginal fluid mixed with semen. Forensic Science International, Volume: 72, Issue 1, Pages 25-33

BAHNAK, B. R., WU, Q. Y., COULOMBEL, L., DROUET, L., KERBIRIOU-NABIAS, D. & MEYER, D. 1988. A simple and efficient method for isolating high molecular weight DNA from mammalian sperm. Nucleic Acids Research, 16, 1208-1208.

BREDBACKA, P., KANKAANPÄÄ, A. & PEIPPO, J. 1995. PCR-sexing of bovine embryos: a simplified protocol. Theriogenology, 44, 167-176.

BROEKHUIJSE, M. L. W. J., FEITSMA, H. & GADELLA, B. M. 2012. Artificial insemination in pigs: predicting male fertility. Veterinary Quarterly, 32, 151-157.

CHEN, Y.-H. & KUO, Y.-H. 2011. Evaluation of different lysis buffers for improving resolution in proteomic analysis of porcine spermatozoa Yu-Hui Chen (, You-Hai Kuo (2), Meng-Ting Chung (, Yu-Fang Chiu (and San-Yuan Huang. J. Chin. Soc. Anim. Sci, 40, 183-189.

FLÉCHON, J. E., DEGROUARD, J., KOPEC̆NÝ, V., PIVKO, J., PAVLOK, A. & MOTLIK, J. 2003. The extracellular matrix of porcine mature oocytes: Origin, composition and presumptive roles. Reproductive biology and endocrinology: RB&E, 1, 124-124.

GARCIA-VAZQUEZ, F. A., GADEA, J., MATAS, C. & HOLT, W. V. 2016. Importance of sperm morphology during sperm transport and fertilization in mammals. Asian J Androl, 18, 844-850.

GARCIA-VAZQUEZ, F. A., HERNANDEZ-CARAVACA, I., MATAS, C., SORIANO-UBEDA, C., ABRIL-SANCHEZ, S. & IZQUIERDO-RICO, M. J. 2015. Morphological study of boar sperm during their passage through the female genital tract. J Reprod Dev, 61, 407-13.

GILL, P., JEFFREYS, A. J. & WERRETT, D. J. 1985. Forensic application of DNA ‘fingerprints’. Nature, 318, 577-579.

HENNEKENS, C. M., COOPER, E. S., COTTON, R. W. & GRGICAK, C. M. 2013. The effects of differential extraction conditions on the premature lysis of spermatozoa. J Forensic Sci, 58, 744-52.

HIRAYAMA, H., KAGEYAMA, S., MORIYASU, S., SAWAI, K., ONOE, S., TAKAHASHI, Y., KATAGIRI, S., TOEN, K., WATANABE, K., NOTOMI, T., YAMASHINA, H., MATSUZAKI, S. & MINAMIHASHI, A. 2004. Rapid sexing of bovine preimplantation embryos using loop-mediated isothermal amplification. Theriogenology, 62, 887-896.

JIANG, Z., ZHANG, X DEKA, R. & JIN, L. 2005. Genome amplification of single sperm using multiple displacement amplification. Nucleic Acids Research, 33, e91-e91.

KHAMLOR, T., PONGPIACHAN, P., SANGSRITAVONG, S. & CHOKESAJJAWATEE, N. 2014. Determination of Sperm Sex Ratio in Bovine Semen Using Multiplex Real-time Polymerase Chain Reaction. Asian-Australas J Anim Sci, 27, 1411-6.

KIRKPATRICK, B. W. & MONSON, R. L. 1993. Sensitive sex determination assay applicable to bovine embryos derived from IVM and IVF. Journal of Reproduction and Fertility, 98, 335-340.

LI, C., SUN, Y., YI, K., LI, C., ZHU, X., CHEN, L. & ZHOU, X. 2011. Detection of the SRY Transcript and Protein in Bovine Ejaculated Spermatozoa. Asian-Australasian Journal of Animal Sciences, 24, 1358-1364.

LOPEZ RODRIGUEZ, A., VAN SOOM, A., ARSENAKIS, I. & MAES, D. 2017. Boar management and semen handling factors affect the quality of boar extended semen. Porcine Health Management, 3, 15.

MAO, S., GOODRICH, R. J., HAUSER, R., SCHRADER, S. M., CHEN, Z. & KRAWETZ, S. A. 2013. Evaluation of the effectiveness of semen storage and sperm purification methods for spermatozoa transcript profiling. Systems biology in reproductive medicine, 59, 287-295.

MARTÍN, I., GARCÍA, T., FAJARDO, V., ROJAS, M., PEGELS, N., HERNÁNDEZ, P. E., GONZÁLEZ, I. & MARTÍN, R. 2009. SYBR-Green real-time PCR approach for the detection and quantification of pig DNA in feedstuffs. Meat Science, 82, 252-259.

POMP, D., GOOD, B. A. GEISERT, R. D. CORBIN, C. J. & CONLEY, A. J. 1995. Sex identification in mammals with polymerase chain reaction and its use to examine sex effects on diameter of day-10 or -11 pig embryos. Journal of animal science, 73, 1408-1415.

RODRIGUEZ-MARTINEZ, H. 2013. Sperm biotechnologies in domestic species: state of the art. Anim Reprod, 10, 268-276.

ROMAR, R., FUNAHASHI, H. & COY, P. 2016. In vitro fertilization in pigs: New molecules and protocols to consider in the forthcoming years. Theriogenology, 85, 125-34.

TORNER, E., BUSSALLEU, E., BRIZ, M. D., GUTIÉRREZ-ADÁN, A. & BONET, S. 2013. Sex determination of porcine embryos using a new developed duplex polymerase chain reaction procedure based on the amplification of repetitive sequences. Reproduction, Fertility and Development, 25, 417-425.

TRIANA, L. R., BABCOCK, D. F., LORTON, S. P., FIRST, N. L. & LARDY, H. A. 1980. Release of Acrosomal Hyaluronidase Follows Increased Membrane Permeability to Calcium in the Presumptive Capacitation Sequence for Spermatozoa of the Bovine and Other Mammalian Species 1. Biology of Reproduction, 23, 47-59.

WIECZOREK, J., KOSENIUK, J., MANDRYK, I. & PONIEDZIALEK-KEMPNY, K. 2015. Piglets born after intrauterine laparoscopic embryo transfer. Pol J Vet Sci, 18, 425-31.

### TITLE 3

### Monoclonal Antibody for Sex Selection

### Field of the Invention 3

This invention relates to a monoclonal antibody that specifically binds to spermatozoa bearing a Y chromosome as well as to methods of using the antibody for sex-specific spermatozoa selection. In one embodiment, the invention concerns a monoclonal antibody that specifically binds sperm surface protein DBY/DEAD of male sperm cells. In another embodiment, the invention concerns a method of collecting and treating semen-containing spermatozoa prior to artificial insemination, to increase the likelihood of obtaining female offspring.

### Background of the Invention 3

The sex of a mammalian offspring is determined by the spermatozoon (sperm) cell. A spermatozoon bearing an X chromosome (‘female sperm cell’) will, upon fertilisation, lead to a female (XX) offspring, whilst a spermatozoon bearing a Y chromosome (‘male sperm cell’) will, upon fertilisation, lead to a male (XY) offspring.

Fertilisation occurs when either a male or female sperm cell penetrates an egg. Engagement with the egg is mediated primarily by sperm surface proteins. These proteins are unique, cell specific, immunogenic and accessible to antibodies. Female and male sperm cells differ in their repertoire of surface proteins. Surface proteins that are unique to male sperm cells include MEA 1, MEA 2, SRY, TSPY and DBY/DEAD. See, for example, WO 2008/067651 and US 2009/0208977, which are incorporated by reference herein.

‘Sex selection’, being the ability to purposively select for male or female offspring, has been hotly pursued in the animal husbandry industry for many years. In particular, the ability to select for female porcine and bovine offspring has been pursued, and different types of techniques have been proposed. Some of the earlier proposed techniques are addressed below, and these have commonalities in the sense that an antibody (polyclonal, monoclonal or partial) against a sex-specific epitope on the surface of mammalian spermatozoa (‘sperm cells’) was targeted.

U.S. Pat. No. 3,687,806 describes the use of antibodies which react with either male or female sperm cells and utilises an agglutination step to separate bound antibodies from unaffected antibodies.

U.S. Pat. No. 4,191,749 describes the use of a male-specific antibody coupled to a solid-phase immunoabsorbant material to selectively bind male sperm cells, while the female sperm cells remain unbound.

U.S. Pat. No. 5,021,244 describes the use of antibodies directed to specific membrane proteins for producing subpopulations enriched in male or female sperm cells.

U.S. Pat. Nos. 6,153,373 and 6,489,092 describe the use of antibodies coupled to magnetic particles for separation of male and female sperm cells.

U.S. Patent Application US 2018/0201667 describes an antibody for bovine semen that separates male and female sperm cells when subjected to flow cytometry.

Canadian Patent Application CA 2610295 describes a polyclonal rabbit antibody that separates male and female boar sperm cells by swim-up and flow cytometry.

However, each of these techniques suffers from disadvantages, including an inability to produce adequate reproducible sex selection results.

### DETAILED DESCRIPTION OF THE INVENTION 3

The inventors have developed an antibody capable of selectively binding to male sperm cells (spermatozoa). The inventors have developed a method of treating spermatozoa and separating male sperm cells from female sperm cells. The inventors have found that, after treatment with the antibody, viable female sperm cells and can be used for artificial insemination in mammals and produce female offspring. Hence, the method increases the probability of producing a mammalian offspring of a desired sex by artificial insemination using antibody-treated semen.

The general method includes the steps of collecting semen from a male donor (e.g., a proven artificial insemination bull or boar), and adding antibody to the semen in a ratio so as to enable inactivation, agglutination, removal or death of spermatozoa carrying the unwanted male sperm cells within or from the semen (ie. ‘treated semen’). Mammals can then be artificially inseminated with the treated semen to produce female offspring.

According to a first aspect of the present invention, there is provided a monoclonal antibody or fragment thereof which specifically binds to and/or is raised against a surface protein of a mammalian male spermatozoon (male sperm).

The antibody is non-naturally occurring. The antibody is produced by recombinant means. The antibody can be in any suitable form, such as in an isolated, purified or substantially purified form.

The terms “monoclonal antibody” and “monoclonal antibodies” as used herein refer to a preparation of antibodies of single molecular composition. A monoclonal antibody displays a single binding specificity and affinity for a particular epitope of a target antigen.

The “antibody” herein shall be interpreted as encompassing any specific binding factors having the binding domain with required specificity. Therefore, this term encompasses homogeneous antibody segments, derivatives, and humanized antibodies thereof, as well as the antibody's functional equivalents and homologues, and also includes any polypeptide having antigen binding domains, either natural or synthetic. Examples of the antibody are immunoglobulin subtypes (e.g. IgG, IgE, IgM, IgD and IgA) and subtypes and subclasses thereof; it may further be a segment comprising antigen binding domains, such as Fab, scFv, Fv, dAb and Fd; and diabodies. Chimeric molecules fused to another polypeptide and comprising antigen binding domain or equivalents thereof are also included.

The monoclonal antibody according to the present invention may be, for example, monovalent or single-strand antibody, double-strand antibody, chimeric antibody, humanized antibody, and derivatives, functional equivalents and homologues of the above antibodies, and may further comprise antibody segments and any polypeptide comprising antigen binding domains.

Antibodies may be modified through a variety of ways, may produce other antibodies or chimeric molecules that retain the original antibody's specificity using DNA recombinant technology. This technology may introduce DNA that encodes immunoglobulin variable domains or CDRs of the antibody into constant domains or constant domains plus framework regions of different immunoglobulins. Genetic mutation or other changes may be performed on hybridoma or other cells that produce antibodies, which may change or not change the binding specificity of the produced antibodies.

Other than highly variable domains CDR1, CDR2 and CDR3 in heavy chains and light chains, and linker sequences, the remaining part of the monoclonal antibody according to the present invention is framework region. The framework region may be replaced by other sequences provided that the three dimensional structure required by the binding is not affected. The molecular basis of the antibody's specificity primarily comes from its highly variable domains CDR1, CDR2 and CDR3, which are key positions to bind with antigens. To maintain the preferred binding specificity, CDR sequences should be retained as much as possible. However, it might be necessary to change some amino acids to optimize the binding specificity. Those skilled in the art may attain this goal through standard practices.

The target antibody may be humanized. Generally speaking, the humanized antibody is an antibody modified through performing amino acid substitution in the framework region of the parent antibody, and compared with the parent antibody, the humanized antibody has lower immunogenicity. Antibodies may be humanized with a number of technologies that are well known in the art. Generally speaking, such humanization methods comprise the identification of appropriate sites through comparing antibody sequences capable of binding identical antigens, and substitution of amino acids on said sites with different amino acids at the same sites of similar amino acids. According to these methods, amino acid sequences of the parent antibody are compared with other associated antibodies (e.g. sequence alignment), thereby identifying variation tolerant positions. Amino acid sequences of variable domains of the parent antibody are typically compared with amino acid sequences in human antibody databases, and a humanized antibody with similar amino acid sequences as the parent antibody is selected. Sequences of the parent antibody and the humanized antibody are compared (e.g. sequence alignment), and amino acids at one or more variation tolerant positions of the parent antibody are substituted by amino acids at corresponding positions in the humanized antibody.

The above-discussed substitution method of variation tolerant positions can be easily combined with any known humanization method, and be easily applied in the production of humanized antibodies comprising CDRs, the CDRs of said antibodies being modified while loyal to the CDR of the parent antibody. Therefore, the present invention further provides humanized monoclonal antibodies comprising a plurality of CDRs from the modified versions of the parent antibody.

The antibody may be modified through a variety of ways, may produce other antibodies or chimeric molecules that retain the original antibody's specificity using the DNA recombinant technology. This technology may introduce DNA that encodes immunoglobulin variable domains or CDRs of the antibody into constant domains or constant domains plus framework regions of different immunoglobulins. Genetic mutation or other changes may also be performed on hybridoma or other cells that produce antibodies, which may change or not change the binding specificity of the produced antibodies.

The monoclonal antibody used in the present invention may also be prepared with the hybridoma method. Since the DNA sequence that codes the humanized antibody according to the present invention can be obtained through a conventional means known to those skilled in the art, such as the artificial synthesis of amino acid sequences publicized in the present invention or PCR amplification, therefore, said sequence can also be linked into an appropriate expression carrier with the recombinant DNA method and with a variety of methods known in the art. Finally, under conditions suitable for the expression of the antibody according to the present invention, cultivate and transform the obtained host cell, and then those skilled in the art employ a well-known conventional separation and purification means to purify the monoclonal antibody according to the present invention.

When the monoclonal antibody is prepared, it can be purified with any known method in the art for purifying immunoglobulin molecules, for example, chromatography (for example, ion exchange chromatography, affinity chromatography, in particular the affinity chromatography for specific antigens through protein A, and other column chromatography), centrifuge, solubility difference, or any other standard techniques for purifying proteins. In many embodiments, the antibody is secreted from cells into the culture medium, and the antibody is obtained through collecting the culture medium and purification.

The terms “antibody” and “immunoglobulin” may be used interchangeably herein. These terms are well known to those skilled in the art and specifically refer to proteins consisted of one or more polypeptides capable of specifically binding with antigens. One form of the antibody constitutes a basic structural unit of the antibody, which is tetramer. It consists of two pairs of completely identical antibody chains, each pair having a light chain and a heavy chain. In each pair of antibody chains, variable domains of the light chain and the heavy chain are joined together to be responsible for binding with antigens, while the constant domains are responsible for effector functions of the antibody.

Currently known immunoglobulin polypeptides comprise κ and λ light chains, and α, γ (IgG1, IgG2, IgG3, IgG4), δ, ε and μ heavy chains or other equivalents thereof. The immunoglobulin “light chain” (about 25 kDa or about 214 amino acids) in its whole length comprises a variable domain consisted of about 110 amino acids at the NH2-terminal, and a κ or λ constant domain at the COOH— terminal. Similarly, the immunoglobulin “heavy chain” (about 50 kDa or about 446 amino acids) in its whole length comprises a variable domain (about 116 amino acids) and one of heavy chain constant domains, such as γ (about 330 amino acids).

Terms “antibody” and “immunoglobulin” comprise any isoform antibodies or immunoglobulins, or antibody segments that are still specifically bound with antigens, including but not limited to Fab, Fv, scFv and Fd segments, chimeric antibody, humanized antibody, single-strand antibody, as well as fusion proteins having antigen binding portions of antibodies and non-antibody proteins. Said term further comprises Fab′, Fv, F(ab′)2 and/or other antibody segments and monoclonal antibodies capable of specifically binding with antigens.

Antibodies may also exist in a variety of forms, for example, comprising Fv, Fab and (Fab′)2, as well as bifunctional hybrid antibodies (e.g., Lanzavecchia et al., Eur. J. Immunol, 1987; 17, 105), and in the form of single strand (e.g., Huston et al., Proc. NatL Acad. Sci. U.S.A., 1988; 85, 5879 and Bird et al., Science, 1988; 242, 423, which are cited herein as reference). Variable domains of heavy chain or light chain of immunoglobulin consist of three hypervariable domains (also referred to as “complementarity determining region” or CDR). These hypervariable domains are spaced apart by framework regions (FR). The scopes of FR and CDR have been precisely defined (see “Sequences of Proteins of Immunological Interest,” E. Kabat et al., U.S. Department of Health and Human Services, 1991). Amino acid sequences of all antibodies discussed herein are sorted by referring to the Kabat system. Different light chain and heavy chain FR sequences of the same species are relatively conserved. Antibody FRs are used to position and calibrate CDRs. CDRs are mainly responsible for binding with antigen epitopes.

A chimeric antibody is an antibody with constructed heavy chain and light chain genes, in particular an antibody with variable domain and constant domain genes that are genetically engineered and belong to different species. For example, variable domain segments of mouse monoclonal antibody genes are joined to constant domain segments of human antibody, such as γ1 and γ3. Chimeric antibodies can also use genes from other mammal species.

Terms “humanized antibody” and “humanized immunoglobulin” have the same meaning. Compared with the non-humanized form of an antibody, its humanized antibody typically reduces the immunoreaction in the human host.

It should be understood that the antibody designed and produced according to the present invention may replace some conservative amino acids, which have substantially no impact on antigen binding or other functions of the antibody. In other words, amino acids can be mutually substituted in the combinations of gly and ala; val, ile and leu; asp and glu; asn and gln; ser and thr; lys and arg; phe and tyr. Amino acids not in the same group are “substantially different” amino acids.

In some embodiments, the affinity between an antibody and its target spot is represented by Kd (dissociation constant), which is lower than 10⁻⁶ M, 10⁻⁷ M, 10⁻⁸ M, 10⁻⁹ M, 10⁻¹⁰ M, 10⁻¹¹ M or about 10⁻¹² M or lower.

“Variable domain” of an antibody's heavy chain or light chain is the mature region at the N terminal of said chain. All domains, CDRs and residue numbers are defined through sequence alignment and based on existing structural knowledge. Determination and numbering of FR and CDR residues are based on what Chothia and others have described (Chothia, Structural determinants in the sequences of immunoglobulin variable domain. J Mol Biol. 1998; 278, 457).

VH is the variable domain of the antibody's heavy chain. VL is the variable domain of the antibody's light chain, which may comprise κ and λ isotypes. K-1 antibody has the κ-1 isotype, while K-2 antibody has the κ-2 isotype, and Vλ is the variable λ light chain.

Terms “polypeptide” and “protein” may be used interchangeably herein. Both of them refer to polymerized amino acids of any length, which may comprise encoding and non-encoding amino acids, chemically or biochemically modified or derived amino acids and polypeptides having modified peptide skeletons. Said terms comprise fusion proteins, including but not limited to fusion proteins having heterogeneous amino acid sequences; fusion proteins having heterogeneous and homogeneous leader sequences, with or without N-terminal methionine residues; proteins with immunological tags; fusion proteins with detectable fusion partners, for example, fusion proteins that can function as fusion partners, including fluorescent protein, β-galactosidase, fluorescein, etc. By way of example, fusion partner amino acid sequences may assist in detection and/or purification of the isolated fusion protein. Non-limiting examples include metal-binding (e.g. polyhistidine) fusion partners, maltose binding protein (MBP), Protein A, glutathione S-transferase (GST), fluorescent protein sequences (e.g. GFP), epitope tags such as myc, FLAG and haemagglutinin tags.

In this regard, the skilled person is referred to Chapter 15 of CURRENT PROTOCOLS IN PROTEIN SCIENCE, Eds. Coligan et al. (John Wiley & Sons NY 1995-2008) for more extensive methodology relating to chemical modification of proteins.

Polypeptides can be of any length, and the term “peptide” refers to polypeptides of the length of 8-50 residues (e.g. 8-20 residues).

“Corresponding amino acids” refer to amino acid residues at the same positions (i.e. they correspond to each other) when two or more amino acid sequences are compared. Comparison and numbering methods of antibody sequences have been described in detail by Chothia (see above), Kabat (see above), and others. It is known to those skilled in the art (see, for example, Kabat 1991 Sequences of Proteins of Immunological Interest, DHHS, Washington, D.C.) that sometimes one, two or three gaps may be made, and/or 1, 2, 3 or 4 residues or at most about 15 residues (in particular in L3 and H3 CDRs) may be inserted in one or two amino acids of an antibody, thereby completing a comparison.

“Substitutable position” refers to a special position of an antibody, which can be substituted by different amino acids without significantly reducing the antibody's binding activity. Methods to determine substitutable positions and how they can be substituted will be described below in more detail. The substitutable position may also be referred to as “variation tolerant position”.

The antigenic protein or peptide and/or any fragments, variants or derivatives thereof may be produced by any means known in the art, including but not limited to, chemical (peptide) synthesis, recombinant DNA technology and proteolytic cleavage to produce peptide fragments.

Chemical synthesis is inclusive of solid phase and solution phase peptide synthesis. Such methods are well known in the art, although reference is made to examples of chemical synthesis techniques as provided in Chapter 9 of SYNTHETIC VACCINES Ed. Nicholson (Blackwell Scientific Publications) and Chapter 15 of CURRENT PROTOCOLS IN PROTEIN SCIENCE Eds. Coligan et al., (John Wiley & Sons, Inc. NY USA 1995-2008). In this regard, reference is also made to International Publication WO 99/02550 and International Publication WO 97/45444.

Recombinant antigenic proteins or peptides may be conveniently prepared by a person skilled in the art using standard protocols as for example described in Sambrook et al., MOLECULAR CLONING. A Laboratory Manual (Cold Spring Harbor Press, 1989), in particular Sections 16 and 17; CURRENT PROTOCOLS IN MOLECULAR BIOLOGY Eds. Ausubel et al., (John Wiley & Sons, Inc. NY USA 1995-2008), in particular Chapters 10 and 16; and CURRENT PROTOCOLS IN PROTEIN SCIENCE Eds. Coligan et al., (John Wiley & Sons, Inc. NY USA 1995-2008), in particular Chapters 1, 5 and 6.

The surface protein of the male sperm cell can be any suitable type of surface protein. For example, the surface protein can be DBY/DEAD (Asp-Glu-Ala-Asp) box polypeptide 3 (‘DEAD’), male enhanced antigen (MEA’) 1, and MEA 2, sex determining region Y (‘SRY’). Non-limiting examples of further antigens specific to male sperm cells may be found in WO 2008/067651 and US 2009/0208977, which are incorporated by reference herein.

The antibody can bind to and/or can be raised against any suitable portion of the surface protein. Examples of suitable sequences are shown in Table 1C.

TABLE 1C
Peptide sequences based on or derived from
antigens DBY/DEAD, MEA 1, MEA 2 and SRY.

| | Antigenic Peptide Sequences - |
|---|---|
| | SEQ ID NOs: 1-346 (ordered |
| Antigen | consecutively) |
| MEA1 | SEQ ID NO: 1: PTEGTGDWISEEPEEEQTETG |
| | SEQ ID NO: 2: PTEGTQDWYREEPEEEQEETG |
| | SEQ ID NO: 3: PHEGTGDWSNEEPEEEMFETG |
| | SEQ ID NO: 4: PTEGIGLWSSEEPEEEYEMTG |
| | SEQ ID NO: 5: PTEGRGDWSWEEPKHEQSEPG |
| | SEQ ID NO: 6: FTEETGDWSSEEPRTEAEETR |
| | SEQ ID NO: 7: PMEGTGYHSSEEPIIEIMETG |
| | SEQ ID NO: 8: PHEITGDWSWAEPEECQEQTG |
| | SEQ ID NO: 9: PQEGTGDRNVNLPEEEQEAPM |
| | SEQ ID NO: 10: LQRRLEEFEGERERLQRMADSAA |
| | SEQ ID NO: 11: PDEGTGDWSSTYPEREVESTG |
| | SEQ ID NO: 12: PTHGTGWISSAEPSENQAETM |
| | SEQ ID NO: 13: TTIDTGAWVLEEPTELQNYNG |
| | SEQ ID NO: 14: PREDGFDCSSEETAYEQEVTG |
| | SEQ ID NO: 15: WHGYTGPWSSLEKALELVEPW |
| | SEQ ID NO: 16: PHPTTGDWGRRQPILLTEELG |
| | SEQ ID NO: 17: GTCGTGANYSDQYRLCQVEYY |
| | SEQ ID NO: 18: PSKATQTHNTEGWREEMHESC |
| | SEQ ID NO: 19: FEICMGVGSCEEPETEAEYTE |
| | SEQ ID NO: 20: CTCAEGVWVVHTPEEHQGETM |
| | SEQ ID NO: 21: PNEFELDWWSAHEVEEASCRD |
| | SEQ ID NO: 22: ATDLTGNWMEEAEHELQMTIM |
| | SEQ ID NO: 23: CYGGGQDITIFRYILYQEAIG |
| | SEQ ID NO: 24: AMEPTGMAESMPISEVQKMGD |
| | SEQ ID NO: 25: FDAEFQDASETCNEIVQCEDM |
| | SEQ ID NO: 26: FTCGHITGPSNEPERMQMCMG |
| | SEQ ID NO: 27: ITAGPADFSLHPHNAKMNEYY |
| | SEQ ID NO: 28: PKEKGGVVKLDNFMNNQWENI |
| | SEQ ID NO: 29: GVFFTKRQENESRRKQHQPTA |
| | SEQ ID NO: 30: PKMVCMIESERETEEMNCCHV |
| | SEQ ID NO: 31: PDTGRWDWSHEGVDEYSRKIR |
| | SEQ ID NO: 32: PTQGNCARSLTDLNIKQSELK |
| | SEQ ID NO: 33: PGAGCPPASSEEYEGEQEEFG |
| | SEQ ID NO: 34: PHEVDGQGSSCWDEEWSTEGT |
| | SEQ ID NO: 35: PTAGTGDWSSEEPEEEQEETG |
| | SEQ ID NO: 36: PDLINADLSTELQALAAEKTG |
| | SEQ ID NO: 37: PSFGLGDINSGNPKEEVEWWA |
| | SEQ ID NO: 38; PIAPLRDSSIIEDMEEYEAHG |
| | SEQ ID NO: 39: PTQWIPQMEEDENMREQCKAG |
| | SEQ ID NO: 40: PIELTRKCVWGSNDEWQEKPK |
| | SEQ ID NO: 41: PLLGPVPWSQNEPPDEVEAFQ |
| | SEQ ID NO: 42: PTKTCGDPQGIEPQMERYSTV |
| | SEQ ID NO: 43: PKYKYTVWMLQEHIFVQNAMD |
| | SEQ ID NO: 44: PVAMTRDWKRGDMEMEDAEDG |
| | SEQ ID NO: 45: PTFRTFDWIFEEPAADYGFIE |
| | SEQ ID NO: 46: PCERMGVWSSEEPEELQNEYS |
| | SEQ ID NO: 47: PTRDEVMPSSTDPEEEHWKTS |
| | SEQ ID NO: 48: PTMGGGGLSSEEHGWEVEKFM |
| | SEQ ID NO: 49: PIECTVDMCCESPEGWQEETG |
| | SEQ ID NO: 50: PTEVCGDMSSLEPKEVAEETP |
| | SEQ ID NO: 51: PHESDNQRSSESPEEEQRSTG |
| | SEQ ID NO: 52: HVRQRDEWVGRFEILDFRYIVKC |
| | SEQ ID NO: 53: FVELNTEPEDEAMMLIVEMDYHM |
| | SEQ ID NO: 54: QVRRVEYLCGHCEDLQRFAFSIA |
| | SEQ ID NO: 55: AMRRSKEGWNEEYRVQRMSKSAM |
| | SEQ ID NO: 56: LARPCSWSYCWRERLVRMAKCDP |
| | SEQ ID NO: 57: TQTILFEFSGEHHRLIRSSKMVR |
| | SEQ ID NO: 58: CDRRVFKEEGCEHTYQDMNRDKP |
| | SEQ ID NO: 59: LRLMTNLIPGMTTKLSRAAKIAA |
| | SEQ ID NO: 60: LMRTFEESEGERPEDQRTAMSGN |
| | SEQ ID NO: 61: LQRLLEEFHWAVNSVQFFNDCAM |
| | SEQ ID NO: 62: LQRRLEYAKCERFRLVRDDDYEA |
| | SEQ ID NO: 63: WHIPLSENNGCRERLGREAVRCL |
| | SEQ ID NO: 64: GQRRLECFCDTRLRGYCMWDSAA |
| | SEQ ID NO: 65: LQRHSEQTHGKQEHLKRHADDMC |
| | SEQ ID NO: 66: LQRFMEEHSGDRERLPSMNVSKA |
| | SEQ ID NO: 67: EQKSLPEQKGERARLQRMRDSAG |
| | SEQ ID NO: 68: LQRRMEESEREHEFLQRMADSAA |
| | SEQ ID NO: 69: GQERLTEREGEFERQERMHDGRA |
| | SEQ ID NO: 70: LARFLEETEGKEENLQRRADSIK |
| | SEQ ID NO: 71: LQKDREEFEKTRHRLMKMTDHWA |
| | SEQ ID NO: 72: LQNSPEEFAGYRQRCQRMADAAA |
| | SEQ ID NO: 73: LQRRLEEWNGERHRLHRMEDIMV |
| | SEQ ID NO: 74: WQRRLSEFRREREETWRMAYSAA |
| | SEQ ID NO: 75: TQRKLEEFEGERERCSKMAVSVA |
| | SEQ ID NO: 76: LQRRLEDFDWSLQRLQRMADSAA |
| | SEQ ID NO: 77: LQRRLEEFEWERHRLGMMACSGA |
| | SEQ ID NO: 78: LQRRLELFEGEYERLQVMALSAA |
| | SEQ ID NO: 79: LIRRLEEFEGERERLQRPADSGA |
| | SEQ ID NO: 80: LQKRDEEFEGERERLQRMADTAA |
| | SEQ ID NO: 81: LQRRLEEFEGERERLQRMATSDA |
| | SEQ ID NO: 82: LARRLEEFEGERERLQRMADSAA |
| | SEQ ID NO: 83: NELYGEEAADESENAQRFQDTAH |
| DBY but | SEQ ID NO: 84: EMSSHIMTQAGVQWPDLGSLEV |
| also | SEQ ID NO: 85: EMESHSVRQAVVQWWDCGSLEV |
| known | SEQ ID NO: 86: ENESHSVTIAGEQWPDMGRLEV |
| as DEAD | SEQ ID NO: 87: EMESWSVPQAGVQVPFVGSLED |
| | SEQ ID NO: 88: EMETHSDLQALQQWGDHGSLEV |
| | SEQ ID NO: 89: EMFSHSQTQLNTQWPDLGTHEV |
| | SEQ ID NO: 90: ESESRSVTQDRRVWPFLGSLEP |
| | SEQ ID NO: 91: EMASYSVVQAGVQWPSFGDNEF |
| | SEQ ID NO: 92: ELESMEVITGGVQWPSWYKLWV |
| | SEQ ID NO: 93: EMESHSCMQPGHHWNDRGLNPQ |
| | SEQ ID NO: 94: EHFEQSVTQAGHGWSDWGSMEE |
| | SEQ ID NO: 95: ESVSTAVTQAGISWPELFEGGR |
| | SEQ ID NO: 96: EMYIHSVTCNGDQRRCLGSGDQ |
| | SEQ ID NO: 97: EMVSFWVRFIYVMWPVLMSCSY |
| | SEQ ID NO: 98: EHEYYSVCQAGVVPWDSGLREV |
| | SEQ ID NO: 99: EMESTMVNGRPWQWYKLCHLEI |
| | SEQ ID NO: 100: EMESHSVTQAGVQWPDLGSLEV |
| | SEQ ID NO: 101: EMSCHYVKQSPNSWDDDASYEV |
| | SEQ ID NO: 102: EMGGHSVLQAGVQSENLLCWLS |
| | SEQ ID NO: 103: EMEHLLQRFCYPQFLDTNSHID |
| | SEQ ID NO: 104: EMDSCDVCEQGLRWKDAGSLTR |
| | SEQ ID NO: 105: EFERCHVTAAGEHKCDACSLEN |
| | SEQ ID NO: 106: EMCQHKVHDGCVQRCKLAHVRV |
| | SEQ ID NO: 107: EFPTTIVGQAMRQIMMTQCLNP |
| | SEQ ID NO: 108: EAAVVSVHCICVKLPDRQQKCV |
| | SEQ ID NO: 109: ESCKHTQNQSAYTWPSPHWGEV |
| | SEQ ID NO: 110: EMNLPSMNLMCVSCLDPCSIGV |
| | SEQ ID NO: 111: EMDEHSNTVALMQEFKSWIYTA |
| | SEQ ID NO: 112: EMCFHCGRQTGGQMQSQLSLEV |
| | SEQ ID NO: 113: ECGYRSVTPFWEQEMNHCSLHG |
| | SEQ ID NO: 114: ECISAPQMRVYGQQPFAYNLEE |
| | SEQ ID NO: 115: EMTSIIWMCSWAQMKDDMDLSV |
| | SEQ ID NO: 116: ENGNCENSKSGQQDSWVQQLEL |
| | SEQ ID NO: 117: ETYPVYLVQRQSNIEDLVQVLQ |
| | SEQ ID NO: 118: EMPSFRPTMYRVRESVYRAANK |
| | SEQ ID NO: 119: EGNNPKLTFAIVQWPHDESTRW |
| | SEQ ID NO: 120: EMKQIQVQEFGKCWQRATSHPV |
| | SEQ ID NO: 121: EKVPVMATYTGAVWMRGGKEIV |
| | SEQ ID NO: 122: ESEFHEVEQAVKKCGFVATLSV |
| | SEQ ID NO: 123: NMSAAVTFSASWQTPFRAPFPG |
| | SEQ ID NO: 124: EMESHSVTQAGVQMPDLGILEV |
| | SEQ ID NO: 125: YLHSCVVNQEWNQLFYLWTLLY |
| | SEQ ID NO: 126: TYEAHGVQTEYCRSNISQWDRD |
| | SEQ ID NO: 127: LCEHKVTGDEETDASNGTCAEP |
| | SEQ ID NO: 128: IMDSFSIKITDWVSPDSDEVKN |
| | SEQ ID NO: 129: NQWAWFVPVSSQPSYLEYRKEV |
| | SEQ ID NO: 130: EWQQYSVFSLGWMLGVDWGLRR |
| | SEQ ID NO: 131: THYQISVCDLKYPIYDFDYNVV |
| | SEQ ID NO: 132: FDWSHSGFHAGSQMVQCFLLWG |
| | SEQ ID NO: 133: GMLSQAHHEAYVWMMKPYFNLM |
| | SEQ ID NO: 134: EMVCWPRTEAIVIKYGVFSREE |
| | SEQ ID NO: 135: WADCQDVTQVYVDGYDHKSWIR |
| | SEQ ID NO: 136: MTASQSQTPLCKQVWDLCEHKL |
| | SEQ ID NO: 137: HHESWSCMPAEVMKTGLAKGAS |
| | SEQ ID NO: 138: IMDATCVTNVGYQDHDEQMINV |
| | SEQ ID NO: 139: RMECHCTGEMGVRVIFIGGTEE |
| | SEQ ID NO: 140: PTTEHTPTDTGYFKHHNASLEH |
| | SEQ ID NO: 141: RLQSLYATQQGYRDPWTGHEEV |
| | SEQ ID NO: 142: EMESQKMFDCRMQGPVVVGEEV |
| | SEQ ID NO: 143: PGESHSQFRNYDLVRYTILQEV |
| | SEQ ID NO: 144: CMMNHSLTAIILQGMRKHELDW |
| | SEQ ID NO: 145: EMFCQSESQACVAIPQTGCHLV |
| | SEQ ID NO: 146: TMHKSNVNKVSVPWCDRGSLTV |
| | SEQ ID NO: 147: EMHCHPYYGARVQYPKLYSDEV |
| | SEQ ID NO: 148: EMEDTWETQLPVRWHDLPDYMY |
| | SEQ ID NO: 149: SMECHSCCQKLMMWRALRSLEA |
| | SEQ ID NO: 150: DMTSMMTRQAFFQWPLPGWADV |
| | SEQ ID NO: 151: YKEYHSVYSPTVVEPPLVSLEW |
| | SEQ ID NO: 152: VCTSQQVLIAVLQMSADIQLEV |
| | SEQ ID NO: 153: IQLSPSVTQAGFVMPDLGSREV |
| | SEQ ID NO: 154: EMEHHSWPLFDVQWHHLNPLEG |
| | SEQ ID NO: 155: DMLEHIITQADVQIPDVGSLEF |
| | SEQ ID NO: 156: EGLSHGFTQAIIQWPDLGSMWV |
| | SEQ ID NO: 157: HMESHSVHQQGVQRPRLGWEEC |
| | SEQ ID NO: 158: MMESHSVASAGVQWKKNPTLIV |
| | SEQ ID NO: 159: EMESHSVAQHGVQWSGNSSMGV |
| MEA2 | SEQ ID NO: 160: LQRHLEEFEGERERLQRMADSAA |
| | SEQ ID NO: 161: LQRRLEEFEGERERLQRVADSKA |
| | SEQ ID NO: 162: LDRRLEEFEGERGRLQRMADSNA |
| | SEQ ID NO: 163: LQRRCEEFHGEYERLQPMADSAA |
| | SEQ ID NO: 164: LERRFEEFWGERVRLQRMADSAL |
| | SEQ ID NO: 165: LQRRLEMFEHGGERLQRYADFAA |
| | SEQ ID NO: 166: LPRRTEEPVGERERLQRCMDSAA |
| | SEQ ID NO: 167: LQKRDAGFEGERERFQRMASSAA |
| | SEQ ID NO: 168: LQRRGEEQEGERERLQRVSDSSS |
| | SEQ ID NO: 169: LQRRGHEFEMERRRLQRMAYHAF |
| | SEQ ID NO: 170: LLNELQVFEFERFHLQRMADSIA |
| | SEQ ID NO: 171: LQGRVNEFNGRRERLDRMIRFAG |
| | SEQ ID NO: 172: LQRYQEEVMNEDERVQEMEDSAH |
| | SEQ ID NO: 173: LERVLEEFYTERHKAPKMAHTFA |
| | SEQ ID NO: 174: LQRCLEEFEDTRCRLGHMPISDA |
| | SEQ ID NO: 175: LIAHLEKFCYERTILMDMIKSAA |
| | SEQ ID NO: 176: LQYCLERFRGLRERWGRSKDSYH |
| | SEQ ID NO: 177: LQRTLMMFEGNRKLMSVMAMYTA |
| | SEQ ID NO: 178: LCRFLKEGKGDREVVVRGAMSKQ |
| | SEQ ID NO: 179: LARLLEEGEWVILRLWELRTGFA |
| | SEQ ID NO: 180: LFRYLKEQNAEPECGVSFYTHAA |
| | SEQ ID NO: 181: LQHRLAEFELYIEERQDPAKRWC |
| | SEQ ID NO: 182: LPRIIAEFEGLREMAGRMANSRP |
| | SEQ ID NO: 183: LTRFYHYFDGMGYRATWYQYGMA |
| | SEQ ID NO: 184: LERRKECTQGDRFPYLMMADQVC |
| | SEQ ID NO: 185: LRRHLTEIPGHNAECQDFKWWKW |
| | SEQ ID NO: 186: LQMRLEWKKHMRNKLVIPDPECA |
| | SEQ ID NO: 187: LHTNCMVEEQIAEPLYAKADYNG |
| | SEQ ID NO: 188: LFRSWWILDTEDEASGSVTPAMT |
| | SEQ ID NO: 189: LFRMTEGGYDCPWHLARTGDSDG |
| | SEQ ID NO: 190: LIRWRYPDEGMRTQAAAMAFQGI |
| | SEQ ID NO: 191: LNTLSVMDEVKGYNMQWEATSAA |
| | SEQ ID NO: 192: QEIRLSVPEMTEVRMSRCTDMAD |
| | SEQ ID NO: 193: CWRPSWECEGLHHGAVRSDHTWT |
| | SEQ ID NO: 194: KQRNGAEFEGFPEQLWRPADYVV |
| | SEQ ID NO: 195: MSPRLWETLVELETTQRCSLTRH |
| | SEQ ID NO: 196: EAGRTFTYKYSLWRGYTFSDSAN |
| | SEQ ID NO: 197: LQRRLEEFEGERERLQRMADSAA |
| | SEQ ID NO: 198: SNGLEEEPFRVRGHTRECFDIAA |
| | SEQ ID NO: 199: DGCRLDEVEGEFRKISPWKDVLA |
| | SEQ ID NO: 200: LFERLAMFEHWYHDKFKAKDSDA |
| | SEQ ID NO: 201: TPRRHNQFYHQKERLQDNGDFMA |
| | SEQ ID NO: 202: LQPLLPIWEGERMRELRMYDSEA |
| | SEQ ID NO: 203: LQLNLEGFCGECPRPVRMAKSAY |
| | SEQ ID NO: 204: HQRRLSFFEVRILNKHRYAHSCA |
| | SEQ ID NO: 205: LSRRLTEQEGTFERSQKFFDMQA |
| | SEQ ID NO: 206: SQMRRKHFIHEREYLYRMLDSIQ |
| | SEQ ID NO: 207: LQRHMEDEEDKRDQVQRMADTAA |
| | SEQ ID NO: 208: KYRNHEEFEGENIRYQVWADHKH |
| | SEQ ID NO: 209: LCRGLEREEGEREEFHRMATQAI |
| | SEQ ID NO: 210: YMRKLEEWHGEIRRHQPLADFAA |
| | SEQ ID NO: 211: TLVGREEFEGEGQMLQRMADSAD |
| | SEQ ID NO: 212: LQRRFENFEGGRERLIRMADFAA |
| | SEQ ID NO: 213: FVRFREEFEGWRERLQRMAPSAA |
| | SEQ ID NO: 214: LQRRVAHFEGERARLQRMADSAA |
| | SEQ ID NO: 215: KQRRLEEFEGECEQLQRIAPSAK |
| | SEQ ID NO: 216: LQRRLEEFEGERYALSRMAASEA |
| | SEQ ID NO: 217: MQRVLAEFEGERQRLQRMADSAA |
| | SEQ ID NO: 218: LQRILEEFEGERERLQREADSAA |
| | SEQ ID NO: 219: LQRRLEEFEDERERLQRMAQSAA |
| | SEQ ID NO: 220: LQRRLEEFFGERERLQRMADSAA |
| MEA2 | SEQ ID NO: 221: RKWLEEQLKQYRVKVQQERSSQ |
| | SEQ ID NO: 222: RAWLEKQLKQYRVKRQQERSSQ |
| | SEQ ID NO: 223: RSWLEEQLKQMRAKRQQERSSQ |
| | SEQ ID NO: 224: RKWSEEQLKQYRVKRQQEWSSR |
| | SEQ ID NO: 225: RKALEQQLKQYRVKRQQSRSSQ |
| | SEQ ID NO: 226: RKWLEEWLKSYRKHRQQERSAQ |
| | SEQ ID NO: 227: RKWSEEQLKGYYSKRQQERMSQ |
| | SEQ ID NO: 228: RRFLEKELKQYRHGRQQERSSR |
| | SEQ ID NO: 229: RDWLETQLKQCIVKRNQNSSMQ |
| | SEQ ID NO: 230: RKWLEGVLKGYLVKSQLESSSG |
| | SEQ ID NO: 231: RFWLKEQLKQYRVKGTVELSRQ |
| | SEQ ID NO: 232: RDWHIEELKQFRVKIQGTRSSN |
| | SEQ ID NO: 233: RKWCEEQLIQFRVFEQGSRYRQ |
| | SEQ ID NO: 234: RKWLEMALKHFKMSRQSEISSQ |
| | SEQ ID NO: 235: RKNLEDQMKGLRVFPGDCGSAQ |
| | SEQ ID NO: 236: RKWLEEQLKQYRVKRQQERSSQ |
| | SEQ ID NO: 237: RKWRDYCLKQYDFDISLERLVR |
| | SEQ ID NO: 238: RKWKGMHDSLYRVKYQGEMNSG |
| | SEQ ID NO: 239: RIWLYWQIKQWFVLGHQKRSSE |
| | SEQ ID NO: 240: RKIIEEINMLYMKKRIDEHSSQ |
| | SEQ ID NO: 241: RKWVEEMLRDGFVKNARWFGPD |
| | SEQ ID NO: 242: RVWEALRRKGERYSAQQHSSSE |
| | SEQ ID NO: 243: RILAIERKGRYDDKTQQQDYFT |
| | SEQ ID NO: 244: RKHRSMCLPQNRCLMGSEDIQQ |
| | SEQ ID NO: 245: RHYCFEIGKQCQHKMANASCPM |
| | SEQ ID NO: 246: RSDIEPHFSAYDFTDKDNCSHQ |
| | SEQ ID NO: 247: RECLERFMVDYDMCDHQARSGQ |
| | SEQ ID NO: 248: RKYLIEILKLFRPWAQQCRHHL |
| | SEQ ID NO: 249: RIYTLDCIKQQRQFTQWGMSQA |
| | SEQ ID NO: 250: RGRDCCAINQKHNNFNVEPCSS |
| | SEQ ID NO: 251: RELLEAQVNQHDYKYLQQQSFQ |
| | SEQ ID NO: 252: RKELRSWSAWYKPEEEQSRSCI |
| | SEQ ID NO: 253: RGQQMEHLRRCLPIRQAEYCDA |
| | SEQ ID NO: 254: HIKCEPMPKQCMVKASMRRYSN |
| | SEQ ID NO: 255: DFGVWEWGKQTPYKRQQWPLDD |
| | SEQ ID NO: 256: TRWSEGQAKFWLNQIVQEWSST |
| | SEQ ID NO: 257: GKWAGITGDPERVESQADFFVP |
| | SEQ ID NO: 258: RKTFEGWLYPYNTKIFEERSQG |
| | SEQ ID NO: 259: NKWLMMIGYNYDLCEYHECNDR |
| | SEQ ID NO: 260: QKWWEEYLTQEDVGRWQKASYW |
| | SEQ ID NO: 261: QKWSEYQHHVYEGPRYEMFFQR |
| | SEQ ID NO: 262: FFMLEVKLETMRAGHQPEDETW |
| | SEQ ID NO: 263: MKVLGEEVFQYRVINGQQCSYT |
| | SEQ ID NO: 264: GTWGPEQGKMYRGKFRQNASYQ |
| | SEQ ID NO: 265: MKWDIAQLKTKCVDHGTNPCSN |
| | SEQ ID NO: 266: SAWLTEQLVQYRVKGQAEKSSQ |
| | SEQ ID NO: 267: LCWHEEYLKQCRVKRQICNSLP |
| | SEQ ID NO: 268: RCWGEEQDKQLAVSRQDEIMQQ |
| | SEQ ID NO: 269: EKQLEKETHQLSVKRQQPRLNF |
| | SEQ ID NO: 270: EKKEENQLKMNPAKRQPERSVR |
| | SEQ ID NO: 271: HKTTEEWLKQYWVKVQQERRSQ |
| | SEQ ID NO: 272: TDWFNEQLKVYREKRQQERTHQ |
| | SEQ ID NO: 273: RKQAEDQLAQYRVKRWQERSAI |
| | SEQ ID NO: 274: VKFPEEQQGMYRWKRQFERSSQ |
| | SEQ ID NO: 275: GKWLEEDFKITRVKRTQERSSQ |
| | SEQ ID NO: 276: RKWLEEQQQLYRVTRGQGRSSQ |
| | SEQ ID NO: 277: RKWLEEQGQGYRVERQQEWSSQ |
| | SEQ ID NO: 278: RKWLEEQLLWHEVKRQQERSSQ |
| | SEQ ID NO: 279: RKWLEEQLKSYKVKRQQERSHQ |
| | SEQ ID NO: 280: RKWLELQLKQYRVKRQQERYSQ |
| | SEQ ID NO: 281: RKWLEEQLKQYRVHRQQERSSQ |
| SRY | SEQ ID NO: 282: RDQRRKMVLENPRMRNSEISKQ |
| | SEQ ID NO: 283: RDQRRQMALENIRMRNSEISKQ |
| | SEQ ID NO: 284: RDQRRRMALENPRMRWSNISKQ |
| | SEQ ID NO: 285: RDQRRKMRLENPRMGNSEISWY |
| | SEQ ID NO: 286: RDQRRKVLREPPRMRNSEISKQ |
| | SEQ ID NO: 287: RDQRSKFCLECPRMRNSCISKR |
| | SEQ ID NO: 288: RDQRRLMAFENPRMKNHELSKA |
| | SEQ ID NO: 289: RMDRIKMAKEHPQSRNSEISKQ |
| | SEQ ID NO: 290: RDQRGKMTEENPCMRNFEISKW |
| | SEQ ID NO: 291: RDQYRKMRLENIFRRMSEIQKI |
| | SEQ ID NO: 292: RNQRRGQAPENYYMRNSDISDQ |
| | SEQ ID NO: 293: RPQRSEQSLENPRMRYDEGSKW |
| | SEQ ID NO: 294: RDTRKTDALEHPRMRNREGSRQ |
| | SEQ ID NO: 295: RNHRRKMRLLNPRMRMSEIQGA |
| | SEQ ID NO: 296: RDYRTKMSLIVPRQRYYEIWKM |
| | SEQ ID NO: 297: RMKRRFCDVENMRMRGYLISEI |
| | SEQ ID NO: 298: RIMRYSGAHKNPHMWRSEYSNQ |
| | SEQ ID NO: 299: RGQMRRTWCENWQTRSSRIRPQ |
| | SEQ ID NO: 300: RDNRRMMALTYPPGRNKWKLDK |
| | SEQ ID NO: 301: RPQHDEMLIINPIMRDSNLTKQ |
| | SEQ ID NO: 302: RDKRRKMPNVNEQCQDAQISLL |
| | SEQ ID NO: 303: RDQKAEQDNENPKMRMVECHGQ |
| | SEQ ID NO: 304: RDEGDKVLDLCNRMMNSRKMIQ |
| | SEQ ID NO: 305: RDVRMKMTRPHCRRRCGEASVV |
| | SEQ ID NO: 306: RKQHYSPSLENKCARKTCQSKI |
| | SEQ ID NO: 307: RLIRCLFCLEMPRIYIPEIHWL |
| | SEQ ID NO: 308: RLIRCLFCLEMPRIYIPEIHWL |
| | SEQ ID NO: 309: RCNWRKCNEEPPNYMHSMFGCQ |
| | SEQ ID NO: 310: RDQRRKMALENPRMRNSEISKQ |
| | SEQ ID NO: 311: RQCRKRWRDNNNPMACSYIRKQ |
| | SEQ ID NO: 312: REQKRPVDVETDFTLRTPHKKK |
| | SEQ ID NO: 313: RHQKVPDAIENPRMRWNGWDTA |
| | SEQ ID NO: 314: RIFRRKVYLERNSYRGDWIWTR |
| | SEQ ID NO: 315: RKQGRPMDCMYPNMHRGYHMHP |
| | SEQ ID NO: 316: RYKSPMTLINRGQIRDTPCSDR |
| | SEQ ID NO: 317: CRQDDIKLLHEGEMEKGRLWKH |
| | SEQ ID NO: 318: RDARCWWTTHGYHHGNCEWYLK |
| | SEQ ID NO: 319: DDQDRKMYTDAPINRLRKALKQ |
| | SEQ ID NO: 320: CRADSWKAKEYPENRPSEIPIQ |
| | SEQ ID NO: 321: RDAYRPVAVHNCMMRMGMIWAE |
| | SEQ ID NO: 322: HTQRNFCFIENTQYWNLEDSWT |
| | SEQ ID NO: 323: HDQRRDHRVGVPRMERGTPAKK |
| | SEQ ID NO: 324: RMFCRHVAYDLPRMRFSYISVQ |
| | SEQ ID NO: 325: RDMIREQAQEKPRLRLSHRWKQ |
| | SEQ ID NO: 326: YDPRINYPLEHIRMSNPEIAKE |
| | SEQ ID NO: 327: EDARVNMALELARPRKSFRMSH |
| | SEQ ID NO: 328: FKQRRFMAMEEPLSRLSERHSC |
| | SEQ ID NO: 329: VTERRKMALKFPPDDAVQISHQ |
| | SEQ ID NO: 330: RPQRRYTGEYNVRFRCEEISHA |
| | SEQ ID NO: 331: TKQTFKMAVGNGTSRNVEINKA |
| | SEQ ID NO: 332: RDQRRVVALVKANHYASLIAKG |
| | SEQ ID NO: 333: RKQKNKMAKENPRMRHSETNKP |
| | SEQ ID NO: 334: RDQRQPMNLWPAAMRNSRIYRQ |
| | SEQ ID NO: 335: RDQQRKKALENPRHRNSAKHTK |
| | SEQ ID NO: 336: NDQRRKFAWGPPRMRNEPISSE |
| | SEQ ID NO: 337: RRQRRKMAWEQPRMQTSVIWRD |
| | SEQ ID NO: 338: SDQRMHMALENARWRNSYIWKQ |
| | SEQ ID NO: 339: RDQGRKMCLENPRQRNKPIKKQ |
| | SEQ ID NO: 340: RDQRMLMTLENPRMRNRAYSWQ |
| | SEQ ID NO: 341: RDQTRAAALENPRMRYSEISKY |
| | SEQ ID NO: 342: RDQRRKNALEPPRMRHSECSKQ |
| | SEQ ID NO: 343: RDQRRKMGLEQPRMRNSEIMKQ |
| | SEQ ID NO: 344: RDQRRKMALENPKMSNSEISMQ |
| | SEQ ID NO: 345: RDQRRKMALENPKMSNSEISMQ |
| | SEQ ID NO: 346: RDQRRKMAKENPRMRNSEISKQ |

In some embodiments the surface protein is DBY/DEAD.

In some embodiments the antibody specifically binds to DBY/DEAD.

In some embodiments the antibody is raised against DBY/DEAD or an antigenic portion thereof.

In some embodiments the antibody is raised against the amino acid sequence of any one of SEQ ID NOs: 347, 352 and 84 to 159 or a portion thereof.

In some embodiments the antibody binds to an epitope of the amino acid sequence of any one of SEQ ID NOs: 347, 352, 353, 354, 355 and 84 to 159.

In some embodiments the antibody binds to the epitope of any one of SEQ ID NOs: 353, 354 and 355, but preferably 353.

In some embodiments the antibody binds to the epitope of any one of SEQ ID NOs: 353, 354 and 355, or an epitope have one or more amino acid substitutions.

In some embodiments the antibody binds to an epitope having a core sequence EMESH (derived from SEQ ID NO: 353), or an epitope have one or more amino acid substitutions.

In some embodiments the antibody heavy chain variable domain comprises the amino acid sequences of CDR1 (SEQ ID NO: 363), CDR2 (SEQ ID NO: 364) and CDR3 (SEQ ID NO: 365).

In some embodiments the antibody light chain variable domain comprises the amino acid sequences of CDR1 (SEQ ID NO: 360), CDR2 (SEQ ID NO: 361) and CDR3 (SEQ ID NO: 362).

In some embodiments the antibody heavy chain variable domain comprises the amino acid sequence of SEQ ID NO: 350 or its heavy chain variable domain is derived by substitution, deletion or addition of one or several amino acids of the amino acid sequence shown by SEQ ID NO: 350, has at least 95%, 96%, 97%, 98% or 99% identity with SEQ ID NO: 350, and said antibody has activity of specifically binding with the surface protein.

In some embodiments the antibody heavy chain variable domain is encoded by the nucleotide sequence of SEQ ID NO: 348.

In some embodiments the antibody light chain variable domain comprises the amino acid sequence of SEQ ID NO: 351 or its light chain variable domain is derived by substitution, deletion or addition of one or several amino acids of the amino acid sequence shown by SEQ ID NO: 351, has at least 95%, 96%, 97%, 98% or 99% identity with SEQ ID NO: 351, and said antibody has activity of specifically binding with the surface protein.

In some embodiments the antibody light chain variable domain is encoded by the nucleotide sequence of SEQ ID NO: 349.

Although DBY/DEAD is of human origin, since it a protein that is highly conserved across different mammalian species, the antibody is capable of binding to DBY/DEAD of different mammalian species, including bovine and porcine species.

The term “mammal” includes, but is not limited to, humans, farm animals, livestock, laboratory animals, companion animals and pets. The term includes, but is not limited to, pigs, cattle, horses, donkeys, dolphins, guinea pigs, hamsters, mice, rats, dogs and cats. Preferably, the mammal is porcine or bovine.

According to a second aspect of the present invention, there is provided a composition comprising an effective amount of the monoclonal antibody of the first aspect and an acceptable carrier, diluent or excipient.

The composition can be, for example, in the form of a pharmaceutical composition, in the form of a composition for veterinary use, or in a form for research purposes (e.g. reagent or research tool).

Acceptable carriers, diluents or excipients are not detrimental or harmful to the sperm. A useful reference describing acceptable carriers, diluents and excipients is Remington's Pharmaceutical Sciences (Mack Publishing Co. N.J. USA, 1991), which is incorporated herein by reference.

In some embodiments, the acceptable carrier, diluent or excipient is a semen extender. A semen extender or diluent is typically an aqueous solution used to increase the volume of the mammalian ejaculate. A semen extender will typically supply nutrients for metabolic maintenance of the sperm cell (e.g., glucose), afford protection against cold shock (e.g., BSA), maintain the pH (e.g., bicarbonate, Tris, Hepes) and osmotic pressure (e.g., NaCl, KCl) of the medium and inhibit microbial growth (e.g., antibiotics). The semen extender can include any of those that are known in the art, including commercially available semen extenders (e.g. Androstar Boar Semen Extender (BSE) from Minitube). A semen extender is typically formulated to be suitable or compatible for the particular species from which the sperm is derived.

According to a third aspect of the present invention, there is provided a reagent, kit or chip comprising the monoclonal antibody of the first aspect or the composition of the second aspect.

The reagent, kit or chip can comprise one or more of the following: the monoclonal antibody, nucleic acid that encodes said antibody, or eukaryotic cells, prokaryotic cells and viruses that contain said antibody.

Other optional components of the reagents, kits or chips comprise: restriction endonuclease, primer and plasmid, buffer solution, etc. for conducting experiments of antibody activity assay. Nucleic acids of said reagents, kits or chips may further comprise restriction endonuclease sites, multiple clone sites, primer sites, etc. for connection thereof with non-rabbit antibody nucleic acids. All components of said agents, kits or chips may be stored individually in separate containers, or some compatible components may be pre-assembled into a single container as needed.

According to a fourth aspect of the present invention, there is provided use of the monoclonal antibody of the first aspect or the composition of the second aspect as a diagnostic agent, reagent or tool.

In this regard, the monoclonal antibody can be labelled and detected, for example, by radioactive isotopes, enzymes capable of producing assayable substances, fluorescent proteins and biotins. Furthermore, the monoclonal antibody can bind with solid carriers, including but not limited to (polystyrene) plates or beads.

According to a fifth aspect of the present invention, there is provided a method of treating mammalian spermatozoa (‘sperm cells’), said method comprising the step of subjecting mammalian semen containing spermatozoa with the monoclonal antibody of the first aspect or the composition of the second aspect such that the antibody specifically binds to male spermatozoa (“male sperm cells”) of the semen.

Subjecting semen to the monoclonal antibody produces “treated semen”, “treated sperm” or “treated sperm cells”.

According to a sixth aspect of the present invention, there is provided a method of treating mammalian spermatozoa (‘sperm cells’) to increase the probability of female offspring being produced therefrom, comprising the step of contacting the mammalian spermatozoa with the monoclonal antibody of the first aspect or the composition of the second aspect such that the antibody specifically binds to male spermatozoa.

According to a seventh aspect of the present invention, there is provided a method of sexing mammalian semen, comprising the step of contacting spermatozoa of the semen with the monoclonal antibody of the first aspect or the composition of the second aspect such that the antibody specifically binds to male spermatozoa of the semen.

According to an eighth aspect of the present invention, there is provided a composition comprising semen or spermatozoa when treated with the monoclonal antibody of the first aspect or the composition of the second aspect.

According to a ninth aspect of the present invention, there is provided a method of artificially inseminating a mammal to increase the probability of female offspring produced therefrom, comprising the step of administering to the mammal:

(i) an effective amount of the monoclonal antibody of the first aspect;

(ii) an effective amount of the composition of the second aspect; or

(iii) an effective amount of the composition of the eighth aspect,

so as to artificially inseminate said mammal.

According to a tenth aspect of the present invention, there is provided a conjugate of the monoclonal antibody of the first aspect or the composition of the second aspect and male spermatozoa (“male sperm cells”).

According to an eleventh aspect of the present invention, there is provided treated sperm or treated semen comprising a conjugate of the monoclonal antibody of the first aspect and male spermatozoa or the composition of the second aspect and male spermatozoa.

According to a twelfth aspect of the present invention, there is provided use of the monoclonal antibody of the first aspect or the composition of the second aspect for male spermatozoa (“male sperm cell”) selection, wherein said male spermatozoa selection is optionally carried out in bulk semen.

According to a thirteenth aspect of the present invention, there is provided use of the monoclonal antibody of the first aspect or the composition of the second aspect for sexing mammalian semen or spermatozoa.

The sperm or semen is contacted with the monoclonal antibody or the composition for a period of time sufficient to form a conjugate between the monoclonal antibody or composition and a plurality of male sperm cells prior to artificially inseminating one or a plurality of female subjects with said treated sperm or treated semen. For the purpose of description, reference is made to a range of 5 to 600 minutes, but contact times prior to insemination may be shorter, such as 1, 2, 3, or 4 minutes, or longer, such as 700, 800, 900 or 1000 minutes. The range of 5 to 600 minutes includes, of course, 6, 7, 8 minutes as well as all one minute increments between 5 and 600.

The semen or sperm can be either freshly harvested or it can have been previously frozen and subsequently thawed. Preferably, the semen or sperm has been freshly harvested.

Typically, although not exclusively, the semen or sperm has not been washed prior to being contacted with the monoclonal antibody or composition.

The method or use can be performed either in vitro in a semen sample or in vivo by simultaneously or sequentially introducing the monoclonal antibody or composition and the sperm to be treated into the reproductive tract of a female animal. Preferably, the method is performed in vitro so as to ensure a sufficient period of contact between the sperm and the monoclonal antibody or composition to form one or a plurality of conjugates therebetween.

In some embodiments for the aspects of the invention described above, the monoclonal antibody and/or the composition reduces or inhibits the motility and/or activity of the male spermatozoa. Any suitable method for achieving this can be used. Suitable examples include magnetic bead separation, agglutination, filtration and flow cytometry. Preferably, such methods do not reduce or alter the motility, viability and/or activity of the remaining female spermatozoa.

The terms, “reduce” and “inhibit”, as used herein to describe the motility and/or activity of male sperm cells, refer to a reduction in and/or amount or level of such motility and/or activity, when compared to a control sample or further biological sample from a subject. In some embodiments, the motility and/or activity of male sperm cells is reduced if its level of motility and/or activity is less than about 95%, 90%, 80%, 70%, 60%, 50%, 40%, 30%, 20% or 10%, or even less than about 5%, 4%, 3%, 2%, 1%, 0.5%, 0.1%, 0.01%, 0.001% or 0.0001% of the level of male sperm cell motility and/or activity in a control sample or further biological sample from a subject.

Determining sperm motility and/or activity can include any method or combination thereof known in the art. Non-limiting examples of such methods include light microscopy, phase microscopy, computer assisted sperm analysis (e.g., Hobson sperm tracker), the swim up method and migration/sedimentation chambers. In this regard, a measure of sperm motility can be a quantitative or qualitative measure.

In some embodiments for the aspects of the invention described above, the method or use comprises the step of removing the antibody-bound male spermatozoa from treated semen or sperm. Any suitable method for removing the antibody-bound male spermatozoa can be used. Suitable examples include magnetic bead separation, agglutination, filtration and flow cytometry. Preferably, such methods do not reduce or alter the motility, viability and/or activity of the remaining female spermatozoa.

In some embodiments flow cytometry, in vitro fertilisation, quantitative PCR (qPCR) assays, fluorescent antibodies, fluorescent in situ hybridisation (FISH) and/or Protein G covered magnetic beads can be used to check treated semen for antibody-bound male spermatozoa, or whether treated semen is devoid or largely devoid of antibody-bound male spermatozoa, or whether treated semen is enriched for female spermatozoa.

Female spermatozoa selection or the sexing of semen can be performed in bulk. In some embodiments of the methods or uses described above, there is no limit on the type of species and the volume of semen produced. For example, pigs, dolphins and horses produce bulk/high volumes of semen, from 250 mL to several litres, whereas dogs, cattle and other ruminants produce millilitre volumes.

In particular embodiments, the monoclonal antibody or composition can be included in a treatment solution to be added directly to the collected sperm or semen and/or to the reproductive tract of the female animal to be inseminated with optionally one or more antibodies or agents that target male sperm cells.

Additionally, the monoclonal antibody of the first aspect can be used in vitro by, for example, coating collection, storage and other artificial insemination equipment.

Suitably, the sperm preparation comprises a plurality of sperm at a biologically acceptable concentration and/or viability for artificial insemination of the mammal. As would be appreciated by the skilled person, this may vary considerably with, for example, the age and/or species of the mammal to be artificially inseminated. This concentration may be altered by the user according to the morphology and/or motility of the plurality of sperm.

In this context of (i), by “effective amount” is meant an amount or concentration of the monoclonal antibody and/or the composition hereinbefore described sufficient to elicit the desired pharmacological, physiological and/or therapeutic effect/s on the plurality of sperm, so as to increase the probability of female offspring produced upon artificial insemination of said mammal. Thus, an effective amount of a substantially purified antibody or a composition is an amount sufficient to bind to the relevant surface protein on a number of male sperm cells in the spermatozoa and impart inhibitory effects thereon, such as on the activity, function and/or motility of the bound male sperm, to thereby promote an increased likelihood of female offspring being produced from artificial insemination of the treated spermatozoa/treated semen. In this context of (ii), by “effective amount” is meant an amount or concentration of the sperm preparation that is sufficient to produce offspring upon artificial insemination of a female mammal.

The effective amount can vary, depending upon factors such as the age, breed, species, body weight, fertility and general health of mammal, the condition of the sperm, and the manner in which the monoclonal antibody, the composition and/or the sperm preparation is to be administered.

Suitably, the monoclonal antibody and/or the composition and the plurality of sperm are added simultaneously or sequentially. In this regard, the monoclonal antibody and/or the composition may be administered first to the mammal followed by the plurality of sperm. Alternatively, the plurality of sperm may be administered to the mammal prior to the monoclonal antibody and/or the composition.

Suitably, the monoclonal antibody and/or the composition and the plurality of sperm are administered to the female in an amount that a) is safe; b) does not interfere with fertility; c) does not cause any teratogenic effects; and d) is not detrimental to the female's health.

It would be understood that the plurality of sperm used may be either freshly harvested or may have been previously frozen and subsequently thawed. Preferably, the spermatozoa have been freshly harvested.

It is well understood that artificial insemination is the technique wherein spermatozoa/semen is collected from a male mammal and subsequently introduced into a female mammal's reproductive tract at proper time with the help of instruments. With regards to the method of the present invention, the monoclonal antibody and/or the composition and the spermatozoa may be administered to the mammal by any method of artificial insemination known in the art. Typically, the monoclonal antibody and/or the composition and spermatozoa/semen or treated spermatozoa/treated semen are inseminated into the female mammal by placing a portion of it either in into the cervix and/or uterus by mechanical methods at a suitable time (e.g., the female mammal is in oestrus) and under hygienic conditions.

In some embodiments of the methods or uses described above, sperm cell selection or the sexing of semen can be performed in bulk. In some embodiments of the methods or uses described above, there is no limit on the type of species and the volume of semen produced. For example, pigs, dolphins and horses produce bulk/high volumes of semen, from 250 mL to several litres, whereas dogs, cattle and other ruminants produce millilitre volumes. Some embodiments of the invention can be applied to both scenarios.

According to a fourteenth aspect of the present invention, there is provided a hybridoma cell comprising the monoclonal antibody of the first aspect.

According to a fifteenth aspect of the present invention, there is provided one or more isolated, purified or recombinant nucleic acids encoding:

(i) a heavy chain variable domain of the monoclonal antibody of the first aspect;

(ii) a light chain variable domain of the monoclonal antibody of the first aspect;

(iii) CDR1 (SEQ ID NO: 363), CDR2 (SEQ ID NO: 364) and CDR3 (SEQ ID NO: 365) of a heavy chain variable domain of the monoclonal antibody of the first aspect; and/or

(iv) CDR1 (SEQ ID NO: 360), CDR2 (SEQ ID NO: 361) and CDR3 (SEQ ID NO: 362) of a light chain variable domain of the monoclonal antibody of the first aspect.

According to a sixteenth aspect of the present invention, there is provided a monoclonal antibody when raised against the amino acid sequence of SEQ ID NO 347.

Any of the features described herein can be combined in any combination with any one or more of the other features described herein within the scope of the invention.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments

So that the present invention may be more readily understood and put into practical effect, the skilled person is referred to the following non-limiting examples.

### BRIEF DESCRIPTION OF FIG. 3

FIG. 1C. Annotated VH and VL amino acid sequences of monoclonal antibody SdW1 RE5.B7.F9b (also referred to as ‘clone RE5’ or ‘RE5 antibody’), showing framework regions, (FWR), and complementary determining regions (CDR).

FIG. 2C. A homology model built using antibody mAb735 (PDB 3wbd) as template and RE5 antibody as target.

FIG. 3C. Predicted 3D structure of antibody RE5. The CDR loops are circled in the structure.

FIG. 4C. Predicted 3D structure of DBY surface protein (on male sperm cells) coloured according to N/C terminal. N terminal region is blue and C-terminal region is red. The 22-mer peptide is coloured white.

FIG. 5C. Distribution of MCSS minima of three representative groups (Benzene, Acetate ion and Mgua) around the CDR region of the RE5 antibody.

FIG. 6C. Distribution of MCSS minima of functional group and its corresponding amino acid in parenthesis: BENZ (Phe), PHEN (TYR), IMIA (His), ACET (GLU/ASP), and MGUA (ARG).

FIG. 7C. Complex structure of RE5 antibody and DBY protein with predicted binding peptide coloured in orange. Antibody is shown as surface in white. The peptide that was used for developing the RE5 antibody is coloured in blue.

FIG. 8C. Three predicted peptide binders/epitopes/docking sites for monoclonal antibody RE5, shown in underline.

FIG. 9C. Flow diagram showing a process for producing, selecting and characterising the monoclonal antibody. The process includes the following steps: 1. Producing an antibody; 2. Determining the effect of the antibody on semen by agglutination, fluorescent secondary antibodies, FISH or magnetic bead technology; 3. Testing the effect of the antibody on semen using high throughput in vitro fertilisation (HT IVF) or selective lysis and qPCR; and 4. Characterising the antibody by way of sequencing, building a 3-D structure, and determining the epitopes.

TABLE 2C
Listing of SEQ ID NOs.

| SEQ ID | | |
|---|---|---|
| No. | Brief Description | Sequence |
| 1-346 | Peptide sequences | See Table 1C |
| | based on or derived | |
| | from antigens | |
| | DBY/DEAD, MEA | |
| | 1, MEA 2 and SRY | |
| 347 | Antigen SdW1a | EMESHSVTQAGVQWPDLGSLEV |
| | (free peptide) | |
| 348 | Heavy-chain | GAAGTGAAGGTTGAGGAGTCTGGAGGAGGCTTG |
| | variable region (VH) | GTGCAACCTGGGGGATCCATGAAAATCTCCTGTG |
| | of monoclonal | TTGCCTCTGGATTCACTTTCAAGAACTACTGGAT |
| | antibody clone RE5 | GAACTGGGTCCGCCAGTCTCCAGAGAAGGGGCTT |
| | (nucleotide | GAGTGGGTTGCTGAAATTAGATCGAAATCTAATA |
| | sequence) | ATAATGAAAAACATTATGCGGAGTCTGTGAAAG |
| | | GGAGGTTCACCATCTCAAGAGATGATTTTAAAAG |
| | | TAGTGTGTACCTGCAAATGAACAACTTAAGAACT |
| | | GAAGACACTGGCATTTATTACTGTACGGGGGGGA |
| | | CCTTTGACTACTGGGGCCAAGGCACCACTCTCAC |
| | | AGTCTCCTCA |
| 350 | Heavy-chain | EVKVEESGGGLVQPGGSMKISCVASGFTFKNYWM |
| | variable region (VH) | NWVRQSPEKGLEWVAEIRSKSNNNEKHYAESVKG |
| | of monoclonal | RFTISRDDFKSSVYLQMNNLRTEDTGIYYCTGGTFD |
| | antibody clone RE5 | YWGQGTTLTVSS |
| | (amino acid | |
| | sequence) | |
| 351 | Light-chain (kappa) | DVVVTQTPLSLPVSLGDQASISCRSSHSLVHSDGNT |
| | variable region (VL) | YLHWYLQKPGQSPKLLIYKVSNRF |
| | of monoclonal | SGVPDRFSGSGSGTDFTLKISRVEAEDLGVYFCSQT |
| | antibody clone RE5 | THVPPYTFGGGTQLEIK |
| | (amino acid | |
| | sequence) | |
| 352 | DBY(Homo | DBYIDKVVVPAVLALVLVAEAAAAVVEVVTATAEDL |
| | sapiens). Amino acid | VEVMLIFLLGRAFCFSFFFFFEMESHSVTQAGVQWP |
| | translation of Gene | DLGSLEVTLLPQPPKVGLQVGGNMPSSFFSIFNRDG |
| | ID G49469 from | VSPCWPGWSLPPDLMIHTPWPPEVLGLQAATVPGL |
| | NIH. | GSLFFLRVLFFKAFIGE1FLRDTKSNSRFLLLVLCSTE |
| | | KKGINELNFSLNIFLDRWLWRLLQWIWRKLLPGGL |
| | | VGQLN |
| 353 | Actual peptide | SFFFFEMESH |
| | binder/epitope/ | |
| | docking site of DBY, | |
| | recognised by | |
| | monoclonal antibody | |
| | RE5 | |
| 354 | Predicted optimal | FSIFNRDGV |
| | peptide | |
| | binder/epitope/ | |
| | docking site on DBY, | |
| | for a monoclonal | |
| | antibody | |
| 355 | Predicted optimal | SLNIFLDRW |
| | peptide | |
| | binder/epitope/ | |
| | docking site on DBY, | |
| | for a monoclonal | |
| | antibody | |
| 356 | Forward primer | GTTGCACTTTCACGGACGCAGC |
| | specific for | |
| | chromosome 1 | |
| 357 | Reverse primer | CTAGCCCATTGCTCGCCATAGC |
| | specific for | |
| | chromosome 1 | |
| 358 | Forward primer | AATCCACCATACCTCATGGACC |
| | specific for | |
| | chromosome Y | |
| 359 | Reverse primer | TTTCTCCTGTATCCTCCTGC |
| | specific for | |
| | chromosome Y | |
| 360 | Light-chain variable | HSLVHSDGNTY |
| | region (VH) CDR1 | |
| | of monoclonal | |
| | antibody clone RE5 | |
| | (amino acid | |
| | sequence) | |
| 361 | Light-chain variable | KVS |
| | region (VH) CDR2 | |
| | of monoclonal | |
| | antibody clone RE5 | |
| | (amino acid | |
| | sequence) | |
| 362 | Light-chain variable | SQTTHVPPYT |
| | region (VH) CDR3 | |
| | of monoclonal | |
| | antibody clone RE5 | |
| | (amino acid | |
| | sequence) | |
| 363 | Heavy-chain (kappa) | GFTFKNYW |
| | variable region (VH) | |
| | CDR1 of | |
| | monoclonal antibody | |
| | clone RE5 (amino | |
| | acid sequence) | |
| 364 | Heavy-chain (kappa) | IRSKSNNNEK |
| | variable region (VH) | |
| | CDR2 of | |
| | monoclonal antibody | |
| | clone RE5 (amino | |
| | acid sequence) | |
| 365 | Heavy-chain (kappa) | TGGTFDY |
| | variable region (VH) | |
| | CDR3 of | |
| | monoclonal antibody | |
| | clone RE5 (amino | |
| | acid sequence) | |
| 336-373 | PCR primers & | See Table 3B |
| | probes | |

### DESCRIPTION OF EMBODIMENTS 3

Preferred features, embodiments and variations of the invention may be discerned from this Description of Embodiments section which provides sufficient information for those skilled in the art to perform the invention. The Description of Embodiments is not to be regarded as limiting the scope of the preceding Detailed Description of Invention in any way.

Antigen Preparation

Some peptides were custom synthesized and conjugated to Keyhole Limpet Hemocyanin (KLH-peptide). KLH is a metalloprotein found in hemolymph of the giant keyhole limpet (Megathura crenulata). The protein is large and oxygen-carrying with multiple subunits, making it an ideal carrier for the peptides.

Production of Monoclonal Antibodies

Immunisation and Serum Titre: Two Robertsonian mice were immunized via a sub-cutaneous route, 3 times at two-week intervals with a combination of 50 μg of antigen (SEQ ID NO: 347) with an immune adjuvant (Sigma-Aldrich cat #S6322) in combination with methylated CpG. A serum sample was collected from the immunized mice and reactivity to the antigen was tested by ELISA at a dilution of 1:250 and 1:1250 and compared to a pre-immunization sample. Both animals demonstrated a positive titre and were selected for fusion.

Hybridoma Fusion: To generate hybridoma cells the mouse spleen was excised, dissociated into a single cell suspension and fused to SP2/0-Ag14 myeloma cells using polyethylene glycol. The resultant hybridoma cells were grown in Azaserine Hypoxanthine containing medium in 20×96 well tissue culture plates.

Screening: Hybridoma cells were grown for 10 days at which point the number of positive hybridoma colonies was determined and after a further 3 days' incubation an aliquot of antibody supernatant was taken for screening. The supernatant was assayed for reactivity to the antigens SdW1a (free peptide—SEQ ID NO. 347) and SdW1b (KLH-peptide), firstly by antigen microarray using an IgG-specific secondary antibody, followed by supernatant ELISA to confirm any positive antibody-producing clones.

Expansion and Freezing: The highest responding ELISA positive clones were then expanded into a 24 well tissue culture plate for 3-4 days at which point they were expanded to a 6-well tissue culture plate. The cells were seeded at a 1:5 (supernatant wells) and 1:25 (cell wells) ratio. Once the cells had reached 80% confluence they were harvested and cryopreserved in 10% DMSO and stored in liquid nitrogen. Supernatant from each clone was also harvested and frozen at −20° C.

Subcloning: One of the ELISA-positive clones, SdW1 RE5.B7.F9b (‘clone RE5’), selected for sub-cloning was subjected to 2 rounds of serial dilution. After each dilution stage, cells were grown for 4-5 days and single colonies producing antibody positive to the antigen were determined by supernatant ELISA, and the top 2 clones were expanded for further rounds. The final monoclonal cell-lines were expanded into 6-well cell-culture plates for 4-5 days, the supernatant was extracted and frozen down along with the cells.

Isotyping: The supernatant of sub-cloned cell-line was tested by a commercially available assay kit to determine the isotype of the monoclonal antibody being produced. Of the five antibody isotypes, the two most common are IgG and IgM. Of the IgG mAbs, there are five potential subclasses (IgG1, IgG2a, IgG2b, IgG2c and IgG3). Furthermore, each mAb can have either a kappa or lambda light chain. A monoclonal antibody of particular interest, designated clone RE5, had the isotype mouse IgG2a, kappa (IgG2a K).

Sequencing of the monoclonal antibody clone RE5═, isotype: mouse IgG2a, kappa

The heavy-chain variable region (VH) and the light-chain variable region (VL) of the monoclonal antibody clone RE5′ was sequenced. Briefly, antibody sequencing involved the following steps: cells of the cell line were grown; total RNA was purified from those cells; messenger RNA was further purified from the total RNA; cDNA synthesis was performed using VH and VL gene-specific reactions; dGTP-tailing of cDNA was carried out; VH and VL cDNA products were PCR amplified; PCR products were gel purified and cloned into pCR™2.1; colonies screening was undertaken using PCR; PCR products or plasmids of interest were subjected to DNA sequencing; and, DNA sequencing results were analysed.

The heavy-chain variable region (VH) of the monoclonal antibody clone RE5 has the nucleotide sequence of SEQ ID No: 348.

The light-chain variable region (VL) of the monoclonal antibody clone RE5 has the nucleotide sequence of SEQ ID NO: 349.

The heavy-chain variable region (VH) of the monoclonal antibody clone RE5 has the polypeptide sequence of SEQ ID NO: 350.

The light-chain variable region (VL) of the monoclonal antibody clone RE5 has the polypeptide sequence of SEQ ID NO: 351.

The CRDs of the heavy-chain variable region (VH) of the monoclonal antibody clone RE5 have the polypeptide sequence of SEQ ID NOs: 363, 364 and 365

The CDRs of the light-chain variable region (VL) of the monoclonal antibody clone RE5 have the polypeptide sequence of SEQ ID NOs: 360, 361 and 362.

Analysis of the heavy-chain and light-chain antibody variable region gene sequences was performed using the IMGT/V-Quest program (The International Immunogenetics Information System; http://www.imgt.org/IMGT_vquest/vquest). The similarity of clone RE5's VH and VL sequences to (un-rearranged) germline mouse antibody genes is shown in Table 3C.

TABLE 3C
Similarity of Clone RE5 VH and VL to un-rearranged germline
mouse antibody sequences (using IMGT/V-Quest program).

| | V gene and allele | J gene and allele | D region |
|---|---|---|---|
| | (nt identity) | (nt identity) | and allele |
| VL | Musmus IGKV1- | Musmus IGKJ2*01 | N/A |
| (kappa) | 110*01 F (97.96%) | F (94.74%) | |
| VH | Musmus IGHV6- | Musmus IGHJ2*01 | Musmus |
| | 6*02 F (95.58%) | F (91.67%) | IGHD3-3*01 F |

N/A = non-applicable.

The annotated VH and VL amino acid sequences of antibody RES showing framework regions, (FWR), and complementary determining regions, (CDR), are shown in FIG. 1C.

3D Structure of Antibody RE5

Based on the sequence of antibody RE5 (target), a homology model was built using antibody mAb735 (PDB 3wbd) as template, as shown in FIG. 2C.

3D Structure of Antibody RE5

A 3D structure of antibody RE5 was successfully built, as seen in FIG. 3C. Note that the sequence of the target is slightly different from the template and the exact 3D structure of the RE5 antibody was built by manually constructing the side chains of different amino acids.

Prediction of Binding Epitopes of DBY to Antibody

After the sequences of the RE5 VH and VL regions were characterised, the binding regions of the antibody to the protein DBY were determined. The epitopes of antigen that bind the antibody were determined using a method published in (Zhang, W, Zeng, X, Zhang, L, Peng, H, Jiao, Y, Zeng, J, Treutlein, H R, (2013) “Computational identification of epitopes in the glycoproteins of novel bunyavirus (SFTS virus) recognized by a human monoclonal antibody (Mab 4-5)”, J. Comput. Aided Mol. Des. 27:539-550).

Briefly, the following steps were used to identify the potentially binding regions/epitopes of DBY:

1) The RE5 monoclonal antibody sequence was solved. Using the sequence a homology model of the antibody was built.

2) 3D structure of DBY protein was modelled.

3) MCSS mapping of functional groups on the antibody was performed.

4) Identification of the binding peptide sequence pattern from the MCSS minima were done and the pattern over the sequence of protein DRY searched to identify the binding regions of the protein to antibodies.

5) The docking of antibody to DBY protein was performed.

6) Molecular Dynamics Simulation of the antibody-DBY complex in water was predicted.

3D Structure of DBY Protein-Protein

There is no available experimental structure for protein DBY (DBY2 Human male Homo sapiens STS genomic, sequence tagged site; 689 bp genomic DNA. Sex: male. Clone_lib: Human male; Accession: G49469.1; GI: 5114028). Using the nucleotide sequence from NIH (Gene ID G49469), it was first translated into the protein sequence, as seen in SEQ ID No: 352.

DBY is highly conserved in nature. Its amino acid sequence is highly conserved between different mammalian species, including humans, bovine and porcine species. Consequently, the monoclonal antibody RE5 is expected to bind to DBY of most, if not all, mammalian species.

Based on the protein sequence of DBY, modelling of protein structure was carried out using InFold algorithm (McGuffin, L.J., Shuid, A. M., Kempster, R., Maghrabi, A. H. A., Nealon J. O., Salehe, B. R., Atkins, J. D. & Roche, D. B. (2017) Accurate Template Based Modelling in CASP12 using the IntFOLD4-TS, ModFOLD6 and ReFOLD methods. Proteins: Structure, Function, and Bioinformatics, 86 Suppl 1, 335-344, doi: 10.1002/prot.25360; McGuffin, L. J., Atkins, J., Salehe, B. R., Shuid, A. N. & Roche, D. B. (2015) IntFOLD: an integrated server for modelling protein structures and functions from amino acid sequences. Nucleic Acids Research, 43, W169-73; Buenavista, M. T., Roche, D. B. & McGuffin, L. J. (2012); Improvement of 3D protein models using multiple templates guided by single-template model quality assessment. Bioinformatics, 28, 1851-1857; Zhang, W, Zeng, X, Zhang, L, Peng, H, Jiao, Y, Zeng, J, Treutlein, H R, (2013) “Computational identification of epitopes in the glycoproteins of novel bunyavirus (SFTS virus) recognized by a human monoclonal antibody (Mab 4-5)”, J. Comput. Aided Mol. Des. 27:539-550).

FIG. 4C shows the resulting 3D structure of the DBY protein.

MCSS Minima on the Surface of RE5 Antibody

FIG. 5C shows the distribution of MCSS minima of three representative groups (Benzene, Acetate ion and Mgua) around the CDR region of the RE5 antibody. All of the MCSS minima distributions are further shown in FIG. 6C. The BENZ minima which correspond to the side chain of amino acid Phe is located at B1 only. Similar feature was also found for group MGUA(Arg), and PHEN (Tyr). For negatively charged group ACET which corresponds to the side chain of amino acids ASP or GLU, two clusters were identified at B1 and B2, respectively. Similar features were also found for the functional group IMIA (His). Note there is deep cavity between sites B1 and B2, and most of MCSS minima are found. This cavity is likely to be an artefact due to homology modelling of antibody and the minima inside the cavity were thus disregarded.

Binding Peptides of DBY to the RE5 Antibody

Based on the MCSS minima, the sequence pattern of X-Z was used to search the sequence of DBY protein, wherein X=R,Y,F,H,D/E and Z=Y, D/E. FIG. 7C shows the peptide binders (in orange) in DBY, and their location in the docked conformation between the antibody and DBY.

Three predicted peptide binders/epitopes/docking sites for monoclonal antibody RE5 are shown in underline in FIG. 8C and in SEQ ID NOs. 353 to 355. The actual binding site/epitope is SFFFEMESH (SEQ ID NO: 353). The two other epitopes (SEQ ID Nos: 354 and 355) are prime candidates for further antibody generation.

Validation of Antibodies

After the monoclonal antibody clones were produced, the effect of the antibodies on spermatozoa was determined by using phase contrast microscopy to determine agglutination or absence thereof, Fluorescent secondary antibody staining, FISH and magnetic bead separation.

Titration of IgG against semen.

Purified IgG or culture supernatant was diluted to a standard of 25 mg/ml with Androstar Boar Semen Extender (BSE). The antibody was further diluted tenfold and 100 μl volumes per dilution added to wells in an ELISA plate. A standard volume (200 μl) of fresh (<5 days old) boar semen in BSE at a density of 10⁷ cells per ml (equal to 10⁹ cells per dose) was added to the antibody dilutions and kept at 37° C. Each sample was visually checked at 100× magnification over a period of 30 minutes to 4 hours and the percentage of clumping determined. A subjective score of 0 (no clumping), 1+ (<50%), 2+ (50%) and 3+ (100%) clumping of the spermatozoa was developed.

Fluorescent Antibody Test

In order to demonstrate that antibody attached to spermatozoa, the primary antibody was tracked by using a fluorescent (Alexa Fluor 488) goat anti-mouse secondary antibody (Jackson Immunologicals). After treatment with the primary antibody, semen was washed 3× with PBS. Secondary antibody was added at a 1/20 dilution, incubated for 60 minutes at 37° C. followed by 3× PBS washes. Spermatozoa were counted under both light and fluorescent conditions using a Tucson camera and software (Scope Scientific, Brisbane). The ratio of the number of fluorescent spermatozoa versus the total number of spermatozoa under light conditions were used to calculate the % of Y-CCSP (male sperm) and X-CCSP (female sperm) for each treatment. A total of 750 spermatozoa were counted per sample, where possible.

Fluorescent In Situ Hybridisation (FISH)

FISH was used to confirm the fluorescent antibody results and to demonstrate that the fluorescent spermatozoa were Y-chromosome carrying (male sperm cells). This method was a modification of the method described by Parilla, et al. 2003. In short, DNA was extracted from porcine muscle, blood and spermatozoa. PCR was used to amplify two products corresponding to Chromosome I and Chromosome Y, 244 and 377 bp respectively. The primer sequences appear in Table 4C and in the sequence listing.

TABLE 4C
Priming oligonucleotide sequences for
Chromosome 1 and Chromosome Y.

| Target | Direction | Sequence |
|---|---|---|
| Chromo- | Forward | 5′-GTTGCACTTTCACGGACGCAGC-3′ |
| some 1 | | (SEQ ID NO: 356) |
| Chromo- | Reverse | 5′-CTAGCCCATTGCTCGCC ATAGC-3′ |
| some 1 | | (SEQ ID NO: 357) |
| Chromo- | Forward | 5′-AATCCACCATACCTCATGGACC-3′ |
| some Y | | (SEQ ID NO: 358) |
| Chromo- | Reverse | 5′-TTTCTCCTGTATCCTCCTGC-3′ |
| some Y | | (SEQ ID NO: 359) |

The master mix for the PCR reaction consisted of 0.4 μl of a mixture 44 dNTP's (dATP, dGTP, dCTP, dTTP each at 2 mmol/l), 2.5 μl (from 10 pmol μl/l stock) of primers for chromosomes 1 (one) and Y respectively, 50-500 ng porcine DNA, 5 μl of a 10× PCR buffer (100 mmol Tris-HCL 1/1, pH 8.3, 500 mmol KCL 1-1, 15 mmol MgCl2 1-10.01% (w/v) gelatine) and 0.5 μl of 5 U Taq DNA polymerase μl/l. The total volume of the reaction was made up to 50 μl with DNAse free water. A 3-step amplification was used after an initial denaturation at 95° C. for 5 min. followed by 35 cycles of denaturation (95° C., 15 seconds), annealing (60° C., 1 min) and extension (72° C., 15 sec). Finally, an elongation step (72° C., 7 min) completed the cycle.

PCR products were pooled and cleaned up with PicoPure PCR columns (Qiagen), the DNA concentrations determined and used for nick translation. An Abbot nick translation kit was used to incorporate modified deoxyuridine triphosphates (dUTP) into the PCR products. SpectrumGreen™ dUTP was used to mark Chromosome 1 probes and SpectrumRed™ dUTP was incorporated into the Chromosome Y probe. This provided a green fluorescence signal for Chromosome 1 and a red fluorescence signal for Chromosome Y. Components were added to a tube in the order listed for nick translation, after which the tube was briefly centrifuged and vortexed before adding the enzyme (last component): 17.5-x μl nuclease-free water; x μl of 1 μg extracted DNA; 2.5 μl 0.2 mM SpectrumGreen, SpectrumOrange or SpectrumRed dUTP; 5 μl 0.1 mM dTTP; 10 μl dNTP mix; 5 μl 10× nick translation buffer; 10 μl nick translation enzyme, producing a 50 μl total volume.

The samples were incubated at 15° C. overnight. A 10 min, 80° C. step stopped the reaction. The products were once again cleaned up using PicoPure PCR columns (Qiagen), with the final elution buffer consisting of 5 μl Tris-EDTA (0.2 mmol l/l) and 5 μl FISH hybridisation buffer.

Semen samples for FISH were washed twice with KCl (75 mM) and then once with Tris-EDTA (1 M). Three μl of semen suspension were spread on clean glass slides in a 7 mm diameter circle drawn with a carbide pencil on the glass. The slides were air dried and fixed in ice cold methanol:glacial acetic acid (3:1). Before hybridisation the excess fixative was removed by immersing the slides in 2× saline-sodium-citrate (SSC) buffer, dehydrated by passing it through a series of ethanol baths (70%, 80% and 100%) and air dried. The slides were flooded with a 1M NaOH solution for 3 minutes, washed in 2×SSC, dehydrated and air dried. Denaturation was carried out by dropping 2 μl of hybridisation buffer (Vysis, Abbott) on each circle, covered with a glass coverslip and incubated at 75° C. for 5 min.

The probe mixture consisted of green (Chromosome 1), red (Chromosome Y) and hybridisation buffer in equal quantities. This was denatured at 75° C. for 5 min. The glass slides were washed again by immersion in 2× saline-sodium-citrate (SSC) buffer, dehydrated by passing it through a series of ethanol baths (70%, 80% and 100%) and air dried. The denatured probe mix was added (1.2 μl per sample), covered with glass slips and the edges sealed with rubber cement to prevent drying. The slides were once again heated to 75° C. for 5 min, where after it was incubated in a dark moist container at 37° C. overnight. After hybridisation, slides were washed for 2 min in a 0.4×SSC+0.1% NP-40 solution at 75° C., followed by a room temperature wash in 2×SSC+(0.3% NP-40). Dehydration through the ethanol series and air drying completed the process. The slides were counterstained with 5 μl of 4′, 6-diamino-2-phenylindole (DAPI) anti fade solution (Vysis) and examined under a fluorescent microscope with 3 filters. (Brightline full multiband filter set optimised for DAPI, FITC and Texas Red, mounted in cube). All cells contained a green signature indicating the presence of Chromosome 1. This was used as a positive control. Cells containing an additional red (for Chromosome Y) signal were considered Y-CCSP (male sperm cells). Cells containing a green signal only were considered X-CCSP (female sperm cells).

Semen samples were mixed with antibody and the males allowed to settle. When clumping spermatozoa were allowed to sink to the bottom, the top free-swimming spermatozoa were recovered and treated to determine the sex of individual sperm. For each sample an average of 750 cells were counted.

Use of Magnetic Beads to Assess Specificity of Antibodies

Protein G magnetic beads are an affinity matrix for the small-scale isolation and purification of antibodies. We knew that our antibody attached to 50% of sperm as detected by anti-horse or mouse fluorescent antibody. The purpose of this study was to isolate the sperm with antibody attached with the help of magnetic beads and to sex it in order to determine the specificity of the different monoclonal antibody clones for males. FISH was done on the antibody treated sperm retained on the magnetic beads to determine if more males than females were removed by the magnetic beads.

The procedure entailed the following steps:

1. Collecting fresh semen from 2 boars.

2. Counting and determining the volume of antibody to be added.

3. Adding antibody to 2 ml of semen and incubating at 37° C. for 1 hour.

4. Washing semen 3 times with PBS to remove unattached antibody.

5. Mixing 50 μl of bead suspension with 500 μl binding buffer. Vortexing, applying magnetic beads, removing supernatant and repeating this procedure again.

6. Adding 160 μl binding buffer to 50 μl washed semen.

7. Mixing and holding at 4° C. for 30 minutes.

8. Applying magnet to remove supernatant.

9. Adding 500 μl binding buffer, mixing well and removing supernatant; repeating this procedure twice.

10. Storing half of the sample as is for FISH analysis.

11. For the rest of the sample: stripping the semen off by breaking the antibody/semen bond as follows: To the bead pellet from number 11, adding 100 μl of elution buffer, vortexing, incubating 5 minutes at 4° C. Applying magnet, transferring the sperm to a new tube, repeating this procedure and pooling the eluted semen. Adding 40 μl of 1M Tris-HCl (pH 9), continuing with FISH on the collected semen the next morning.

Use of HT IVF to assess effect of antibodies on the skewing of sex ratio after treatment of semen for clone RE5

A total of 828 oocytes were fertilised (n Control=376 and n Treatment=452). The embryos were lysed and the sex of each determined.

Calculation of Volume of Antibody to be Added to Semen

A formula for determining the exact ratio of spermatozoa and antibody per dose of semen was developed. As every single ejaculate of a donor animal is different with regards to motility, viability and total number of spermatozoa per volume, a method had to be developed to standardise the volume per dose in order to optimise the agglutination of the Y spermatozoa.

The optimum antibody to sperm ratio was established using the following formula: The total number of spermatozoa per dose was divided by a factor of 1 or 0.1, or 0.09, or 0.08 or 0.075 or 0.06 or 0.05 or 0.045 or 0.03 or 0.025 or 0.015 or 0.01 to give the mg of antibody per dose. This was then translated to the volume per milliliter per dose. The required volume was added to the spermatozoa at 22° C. or 23° C. or 25° C. or 27° C. or 30° C. or 32° or 33° C. or 35° C. or 37° C. or 38.5° C. or 39° C. The semen was kept at this temperature for 5 to 240 minutes before allowed to cool down slowly to room temperature, filtered, packaged and sent to the farm (for insemination) at 15° C. to 17° C.

With the foregoing in mind, it will be appreciated that an example of how the present invention can be performed in practice is as follows:

Testing in Sows

The ratio of antibody to semen that caused 50% of the spermatozoa to clump was used to inseminate sows. Different numbers of total semen per dose (1×10⁹, 2×10⁹, 3×10⁹ and 4×10⁹) were trialed. Boar semen was collected and immediately mixed with pre-warmed (22° C. or 23° C. or 25° C. or 27° C. or 30° C. or 32° or 33° C. or 35° C. or 37° C. or 38.5° C. or 39° C.) Androstar Boar Semen Extender. The semen was further mixed with the antibody previously diluted in Androstar Boar Semen Extender, all kept at 22° C. or 23° C. or 25° C. or 27° C. or 30° C. or 32° or 33° C. or 35° C. or 37° C. or 38.5° C. or 39° C. The antibody and semen were left to react at 22° C. or 23° C. or 25° C. or 27° C. or 30° C. or 32° or 33° C. or 35° C. or 37° C. or 38.5° C. or 39° C.° for 5 to 240 minutes. The semen was divided into 80 ml doses and left at room temperature to slowly cool down from 22° C. or 23° C. or 25° C. or 27° C. or 30° C. or 32° or 33° C. or 35° C. or 37° C. or 38.5° C. or 39° C.° C. The samples were shipped at 15° C. to the farm, where 2 doses were administered per sow. The semen sample was used within 5 days of collection.

Magnetic Beads

Different clones (of the monoclonal antibodies) had different affinities for the magnetic beads. From 74.5% to 30.5% of the retained samples on the beads were males, depending on the clone tested. The sex of the sperm was determined by using FISH. Results for 13 clones appear in Table 5C.

TABLE 5C
Percentage of male sperm captured by magnetic beads
after treatment of 2 different boar semen samples
with antibodies from each of the clones.
% Males in retention sample (MAG BEADS)
Sensitivity

| Sample no | | | |
|---|---|---|---|
| Mab | Boar1 | Boar 2 | Average |
| RF6 | 72 | 77 | 74.5 |
| RF3 | 72 | 68 | 70 |
| RC6 | 72 | 66 | 69 |
| RF4 | 67 | 68 | 67.5 |
| RE6 | 67 | 68 | 67.5 |
| RE5 | 65 | 66 | 65.5 |
| RB2 | 67 | 61 | 64 |
| RC5 | 67 | 60 | 63.5 |
| RG3 | 60 | 65 | 62.5 |
| RA6 | 58 | 63 | 60.5 |
| RA1 | 59 | 59 | 59 |
| RF2 | 54 | 57 | 55.5 |
| RE3 | 23 | 38 | 30.5 |

Fluorescent In Situ Hybridisation

Determination of the sex of each sperm was done by FISH. Males have a red (Y Chromosome) and green (Chromosome 1) marker. Females have only a green marker, lacking the Y Chromosome. Clumping, fluorescent spermatozoa demonstrated mostly male sperm (Green+Red signals). The percentage of females in the supernatant after semen was treated with various antibodies is shown in Table 6C.

TABLE 6C
The percentage of females in the supernatant after semen
was treated with antibody derived from different clones.
Determination of the sex of each sperm
was done by FISH.

| Antibody clone | % females |
|---|---|
| RB2 | 66 |
| RF4 | 63 |
| RE5 | 63 |
| RE6 | 58 |
| RC5 | 56 |
| RA6 | 54 |
| RF6 | 53.3 |
| RE3 | 52 |
| RG3 | 52 |
| RF2 | 52 |
| RF3 | 46 |
| RA1 | 45 |
| RC6 | 39 |

High Throughput In Vitro Fertilisation

The percentage of female sperm of the treated group versus the percentage of female sperm of the controls for 25 groups of oocytes appear in Table 7C (n Total=828, n Control=376 and n Treatment=452). The mean percentage of females for treatment was 71%, for the control 45%. The mean shift from the control was 25%. The 95% CI for treatment was 0.64-0.78 and for the control 0.17-32.

TABLE 7C
The percentage of female embryos produced with IVF in treatment
versus the percentage of females in the controls for 25 groups
of oocytes. The treatment oocytes were fertilised with semen
to which antibodies were added prior to fertilisation. The
control groups had PBS added prior to fertilisation. The
percentage shift indicates the percentage point increase
towards females between the control and treatment.

| Treatment | % Females in | % Females in | % shift from |
|---|---|---|---|
| Number | Treatment | Control | control |
| 1 | 25 | 13 | 12 |
| 2 | 40 | 13 | 27 |
| 3 | 50 | 17 | 33 |
| 4 | 52 | 14 | 38 |
| 5 | 67 | 67 | 0 |
| 6 | 73 | 67 | 6 |
| 7 | 77 | 44 | 33 |
| 8 | 50 | 17 | 33 |
| 9 | 78 | 58 | 20 |
| 10 | 80 | 80 | 0 |
| 11 | 82 | 40 | 42 |
| 12 | 86 | 25 | 61 |
| 13 | 90 | 75 | 15 |
| 14 | 100 | 94 | 6 |
| 15 | 100 | 94 | 6 |
| 16 | 72 | 54 | 18 |
| 17 | 22 | 0 | 22 |
| 18 | 54 | 7 | 47 |
| 19 | 78 | 25 | 53 |
| 21 | 77 | 20 | 57 |
| 22 | 59 | 45 | 14 |
| 23 | 72 | 54 | 18 |
| 24 | 91 | 91 | 0 |
| 25 | 87 | 43 | 44 |

### TITLE 4

### Materials and Methods including for Sex Selection

### Field of the Invention 4

This invention relates, amongst other things, to the use of the materials and methods described in the earlier sections (the inventions of sections 1 to 3), including in combination with each other, for semen sexing, sex selection, HT-IVF, genetically characterising produced offspring, and/or increasing the likelihood of obtaining desired offspring.

### Background of the Invention 4

One of the criticisms of an immunological approach to semen sexing in the past is “non-consistency”. In fact, there have been many problems with known techniques for semen sexing, sex selection, HT-IVF, genetically characterising produced offspring, and/or increasing the likelihood of obtaining desired offspring.

### DETAILED DESCRIPTION OF THE INVENTION 4

The inventors have now developed materials and methods for minimising one or more of the problems described above.

According to a first aspect of the present invention, there is provided a method comprising the steps of:

1. optionally subjecting spermatozoa to a treatment step;

2. subjecting the spermatozoa of step 1 to a sex selection step so as to select for either female or male spermatozoa of interest;

3. carrying out a fertilisation step using the spermatazoa of interest of step 2 to produce at least one oocyte, blastocyst, ovum, embryonic cell or embryo;

4. selectively lysing the at least one oocyte, blastocyst, ovum, embryonic cell or embryo of step 3 in the presence of spermatozoa so as to selectively release cellular material from the at least one lysed oocyte, blastocyst, ovum, embryonic cell or embryo; and

5. using the released cellular material in at least one downstream application.

The spermatozoa can be in any suitable purified, semi purified or unpurified form. For example, the spermatozoa can be within semen or partially or fully separated from semen. The spermatozoa or semen can be mixed with semen extender, for example. The spermatozoa can be as described in other sections of this patent specification.

Step 1 can comprise, for example, utilising spermatozoa whether specifically subjected to a treatment step or not. Step 1 is optional in that the spermatozoa need not be treated at all. In some embodiments released cellular material originating from the untreated spermatozoa of step 1 is characterised using the at least one downstream application—being checked for genetic changes, for example.

Any suitable treatment step or combination of treatment steps can be used in step 1.

In some embodiments treatment can involve subjecting the spermatozoa to an experimental technique, such as flow cytometry, refrigeration, freezing, long-term storage et cetera. That is, spermatozoa undergoing such treatment may change genetically or otherwise, and these changes can be checked for or characterised using the downstream application.

In some embodiments treatment can involve subjecting the spermatazoa to a mutagen or suspected mutagen, whether chemical or non-chemical in nature. In some embodiments the mutagen or potential mutagen can be a chemical, such as sunscreen, detergent, talc etc or any other chemical that may have a mutagenic effect or suspected mutagenic effect.

In some embodiments the treatment step can involve manipulating the spermatozoa with CRISPR (Clustered Regulatory Interspaced Short Palindromic Repeats gene editing), SMGT (Sperm-Mediated Gene Transfer), TALEN (Transcription Activator-Like Effector Nucleases) or other recombinant technology, or subjecting the spermatozoa to biological molecules, particularly binding molecules, such as antibodies.

In some embodiments the treatment step can involve exposing the spermatozoa to one or more forms of radiation, such as ultraviolet radiation, microwave radiation or heat.

It is to be appreciated that the treatment step can be as described in any other section of this patent specification.

The method optionally includes the step of subjecting the at least one oocyte, blastocyst, ovum, embryonic cell or embryo of step 3 to a treatment step. The treatment step can be as described for step 1 or different from that described for step 1.

The sex selection step of step 2 can be carried out in any suitable way. In some embodiments a sperm-specific antibody can be used. For example, an antibody as described in another section of this patent specification, can be used for sex selection. Likewise, a sex selection method described in another section of this patent specification can be used.

Step 2 can be used to select for either male spermatozoa or female spermatozoa. Preferably a male specific antibody is used to bind to and inactivate male sperm cells, as described in another section of this patent specification.

Fertilisation step 3 can be carried out in any suitable way. Preferably step 3 is carried out using HT-IVF as described in another section of this patent specification. This step includes culturing the fertilised cell as described in another section of this patent specification, if required.

Selectively lysing the at least one oocyte, blastocyst, ovum, embryonic cell or embryo in step 4 in the presence of spermatozoa so as to selectively release cellular material from the at least one lysed oocyte, blastocyst, ovum, embryonic cell or embryo, can be carried out in any suitable way. Preferably step 4 is carried out using the lysis buffer and methodology as described in another section of this patent specification.

As mentioned elsewhere in this specification, “cellular material” is preferably substantially intracellular material that would not have otherwise been released without lysis. The term “cellular material” also includes material that would otherwise have remained membrane bound.

As mentioned elsewhere in this specification, “cellular material” includes within its scope genetic material (all forms thereof, including nucleic acids, polynucleotides and more specifically genomes, genes, gene transcripts, gene products and RNA), proteinaceous material (all forms thereof, including polypeptides, proteins, peptides and amino acids), lipid materials (all forms thereof, including fats and lipids), and carbohydrate materials (all forms thereof). As mentioned elsewhere in this specification, “cellular material” includes within its scope all of the components or structures in cellular systems.

In step 5, any suitable downstream application can be used. As mentioned elsewhere in this specification, a downstream application includes any and all molecular-based methods and procedures. Such methods and procedures can be quantitative, qualitative, for selective characterisation, modification, isolation or amplification et cetera. A downstream application can be a screening test or diagnostic test, to identify or confirm any change/s to the cellular material of the oocyte, blastocyte/blastocyst, ovum, embryonic cell, embryo or spermatozoa.

A downstream application can comprise subjecting the cellular material to the action of at least one exogenously added enzyme, such as a protein-based enzyme or RNA based enzyme.

A downstream application can be for, for example, the study of gene/s (genomics and epigenomics), transcript/s (transcriptomics), protein/s (proteomics), metabolite/s (metabolomics), lipid/s (lipidomics) or interaction/s (interactomics).

Potential downstream applications for the cellular material are described in Wang and Bodovitz, Trends Biotechnol. 2010 June; 28 (6): 281-290, the entire contents of which are incorporated herein by way of cross-reference. Other potential downstream applications are described elsewhere in this specification.

According to a second aspect of the present invention, there is provided a method of characterising genetic change, comprising the steps of:

1. optionally subjecting spermatozoa to a treatment step;

2. subjecting the spermatozoa of step 1 to a sex selection step so as to select for either female or male spermatozoa of interest;

3. carrying out a fertilisation step using the spermatazoa of interest of step 2 to produce at least one oocyte, blastocyst, ovum, embryonic cell or embryo;

4. selectively lysing the at least one oocyte, blastocyst, ovum, embryonic cell or embryo of step 3 in the presence of spermatozoa so as to selectively release cellular genetic material from the at least one lysed oocyte, blastocyst, ovum, embryonic cell or embryo; and

5. using at least one downstream application in respect of the released cellular genetic material so as to characterise genetic change.

As mentioned for the first aspect of the invention, features of the second aspect can be gleaned from any other part of this patent specification.

In some embodiments, the methods described herein may be applicable to any human or non-human animal or mammal in which MEA 1, MEA 2, SRY, DBY/DEAD and/or TSPY may be selectively or specifically expressed by its Y-chromosomal sperm. In particular embodiments, the term “mammal” includes but is not limited to pigs, cattle, horses, donkeys, dogs and cats. Preferably, the species of mammal is porcine or bovine.

In some embodiments of the methods or uses described above, sperm cell selection or the sexing of semen can be performed in bulk.

In some embodiments of the method or uses described above, there is no limit on the type of species and the volume of semen produced. For example, pigs, dolphins and horses produce bulk/high volumes of semen, from 250 mL to several litres, whereas dogs, cattle and other ruminants produce millilitre volumes.

Some preferred embodiments of the invention are illustrated in FIG. 4B of part 2 of this specification.

In some embodiments the method can be used for high-throughput in vitro fertilisation (HT-IVF), as outlined below:

Treat oocytes from pig (or other) ovaries to enhance maturation in a newly developed medium (as described in the other section of this specification to do with IVP).

Fertilise with fresh, extended pig (or other) semen.

Grow out to 8-16 cells→70% of oocytes mature and grow in new procedure, industry standard about 40%.

Lyse oocytes and residual semen differently with a novel lysis solution, allowing oocyte DNA to be extracted leaving spermatozoa intact.

Amplify oocytes DNA using qPCR by REPLI g kit SC Polymerase (Qiagen).

Dilute DNA to detect a range of genotypic traits by real time PCR (qPCR).

Change or absence of gene products can be brought about by treatment of spermatozoa and/or oocytes by:

CRISPR (Clustered Regulatory Interspaced Short Palindromic Repeats gene editing);

SMGT (Sperm-Mediated Gene Transfer) (Rodrigues 2013); TALEN (Transcription Activator-Like Effector Nucleases);

antibodies blocking docking sites on spermatozoa; antibodies blocking docking sites on oocytes;

any chemical such as sunscreen, detergent, talc, etc; or

chemicals that may have a mutagenic effect

Uses:

Determine the ability of test regimes or treatments to change genotype;

Transgenic animals;

In vitro fertilisation; or

Sexing of embryos.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims appropriately interpreted by those skilled in the art.

It will be clearly understood that, if a prior art publication is referred to herein, this reference does not constitute an admission that the publication forms part of the common general knowledge in the art in Australia or in any other country.

As used herein, except where the context requires otherwise, the term “comprise” and variations of the term, such as “comprising”, “comprises” and “comprised”, are not intended to exclude further elements, components, integers or steps but may include one or more unstated further elements, components, integers or steps.

Preferred embodiments of the invention from sections 1 to 4 of the specification are defined in the claims below.

1-11. (canceled)

12. A monoclonal antibody or fragment thereof which specifically binds to and/or is raised against a surface protein of a mammalian male spermatozoon, wherein the surface protein is DEAD/DBY.

13-49. (canceled)

50. The monoclonal antibody or fragment thereof of claim 12, wherein said antibody is raised against the amino acid sequence of SEQ ID NO: 347 (EMESHSVTQAGVQWPDLGSLEV) or an antigenic portion thereof.

51. The monoclonal antibody or fragment thereof of claim 12, wherein the antibody or fragment thereof specifically binds to one of the following epitopes of DEAD/DBY: SFFFFEMESH (SEQ ID NO: 353), FSIFNRDGV (SEQ ID NO: 354) or SLNIFLDRW (SEQ ID NO: 355).

52. The monoclonal antibody or fragment thereof of claim 12, wherein the antibody or fragment thereof heavy chain variable domain comprises the amino acid sequences of CDR1 (SEQ ID NO: 363, GFTFKNYW), CDR2 (SEQ ID NO: 364, IRSKSNNNEK) and CDR3 (SEQ ID NO: 365, TGGTFDY) and/or the antibody or fragment thereof light chain variable domain comprises the amino acid sequences of CDR1 (SEQ ID NO: 360, HSLVHSDGNTY), CDR2 (SEQ ID NO: 361, KVS) and CDR3 (SEQ ID NO: 362, SQTTHVPPYT).

53. The monoclonal antibody or fragment thereof of claim 12, wherein the monoclonal antibody or fragment heavy chain variable domain comprises the amino acid sequence of SEQ ID NO: 350 (EVKVEESGGGLVQPGGSMKISCVASGFTFKNYWMNWVRQSPEKGLEWVAEIRSKSNN NEKHYAESVKGRFTISRDDFKSSVYLQMNNLRTEDTGIYYCTGGTFDYWGQGTTLTVSS) or the heavy chain variable domain of the monoclonal antibody or fragment thereof is derived by substitution, deletion or addition of one or several amino acids of the amino acid sequence shown by SEQ ID NO: 350, has at least 95% identity with SEQ ID NO: 350, and said monoclonal antibody or fragment thereof has activity of specifically binding with the surface protein.

54. The monoclonal antibody or fragment thereof of claim 12, wherein the monoclonal antibody or fragment thereof light chain variable domain comprises the amino acid sequence of SEQ ID NO: 351 (DVVVTQTPLSLPVSLGDQASISCRSSHSLVHSDGNTYLHWYLQKPGQSPKLLIYKVSNR FSGVPDRFSGSGSGTDFTLKISRVEAEDLGVYFCSQTTHVPPYTFGGGTQLEIK) or the light chain variable domain of the monoclonal antibody or fragment thereof is derived by substitution, deletion or addition of one or several amino acids of the amino acid sequence shown by SEQ ID NO: 351, has at least 95% identity with SEQ ID NO: 351, and said monoclonal antibody or fragment thereof has activity of specifically binding with the surface protein.

55. The monoclonal antibody or fragment thereof of claim 12, wherein the monoclonal antibody or fragment thereof is capable of binding to bovine or porcine DEAD/DBY.

56. A method of treating mammalian spermatozoa, said method comprising the step of subjecting mammalian semen containing spermatozoa with the monoclonal antibody or fragment thereof of claim 12 such that the monoclonal antibody or fragment thereof specifically binds to male spermatozoa of the semen.

57. The method of claim 56, comprising the step of quantifying the amount of spermatozoa and then adding an appropriate amount of the monoclonal antibody or fragment thereof so as to optimize binding of the monoclonal antibody or fragment thereof to the male sperm cells.

58. The method of claim 56, comprising the step of combining the monoclonal antibody or fragment thereof with semen extender.

59. The method of claim 56, comprising the step of removing the antibody-bound male spermatozoa from treated semen.

60. The method of claim 59, wherein said removal of the antibody-bound male spermatozoa comprises a technique selected from the group consisting of: magnetic bead separation; agglutination; filtration; and flow cytometry.

61. The method of claim 56, wherein said male spermatozoa binding is carried out in bulk semen.

62. The method of claim 56, wherein the method is used for one or more of the group consisting of: sexing mammalian semen; increasing the probability of female offspring being produced therefrom; and, as a sperm cell pretreatment step prior to artificially inseminating a mammal.

63. A conjugate of the monoclonal antibody or fragment thereof of claim 12 and male spermatozoa.


SEQUENCE LISTING (376 sequences)

SEQ ID NO: 1 - protein, 21 aa, Artificial Sequence
  Synthetic
PTEGTGDWISEEPEEEQTETG

SEQ ID NO: 2 - protein, 21 aa, Artificial Sequence
  Synthetic
PTEGTQDWYREEPEEEQEETG

SEQ ID NO: 3 - protein, 21 aa, Artificial Sequence
  Synthetic
PHEGTGDWSNEEPEEEMFETG

SEQ ID NO: 4 - protein, 21 aa, Artificial Sequence
  Synthetic
PTEGIGLWSSEEPEEEYEMTG

SEQ ID NO: 5 - protein, 21 aa, Artificial Sequence
  Synthetic
PTEGRGDWSWEEPKHEQSEPG

SEQ ID NO: 6 - protein, 21 aa, Artificial Sequence
  Synthetic
FTEETGDWSSEEPRTEAEETR

SEQ ID NO: 7 - protein, 21 aa, Artificial Sequence
  Synthetic
PMEGTGYHSSEEPIIEIMETG

SEQ ID NO: 8 - protein, 21 aa, Artificial Sequence
  Synthetic
PHEITGDWSWAEPEECQEQTG

SEQ ID NO: 9 - protein, 21 aa, Artificial Sequence
  Synthetic
PQEGTGDRNVNLPEEEQEAPM

SEQ ID NO: 10 - protein, 23 aa, Artificial Sequence
  Synthetic
LQRRLEEFEGERERLQRMADSAA

SEQ ID NO: 11 - protein, 21 aa, Artificial Sequence
  Synthetic
PDEGTGDWSSTYPEREVESTG

SEQ ID NO: 12 - protein, 21 aa, Artificial Sequence
  Synthetic
PTHGTGWISSAEPSENQAETM

SEQ ID NO: 13 - protein, 21 aa, Artificial Sequence
  Synthetic
TTIDTGAWVLEEPTELQNYNG

SEQ ID NO: 14 - protein, 21 aa, Artificial Sequence
  Synthetic
PREDGFDCSSEETAYEQEVTG

SEQ ID NO: 15 - protein, 21 aa, Artificial Sequence
  Synthetic
WHGYTGPWSSLEKALELVEPW

SEQ ID NO: 16 - protein, 21 aa, Artificial Sequence
  Synthetic
PHPTTGDWGRRQPILLTEELG

SEQ ID NO: 17 - protein, 21 aa, Artificial Sequence
  Synthetic
GTCGTGANYSDQYRLCQVEYY

SEQ ID NO: 18 - protein, 21 aa, Artificial Sequence
  Synthetic
PSKATQTHNTEGWREEMHESC

SEQ ID NO: 19 - protein, 21 aa, Artificial Sequence
  Synthetic
FEICMGVGSCEEPETEAEYTE

SEQ ID NO: 20 - protein, 21 aa, Artificial Sequence
  Synthetic
CTCAEGVWVVHTPEEHQGETM

SEQ ID NO: 21 - protein, 21 aa, Artificial Sequence
  Synthetic
PNEFELDWWSAHEVEEASCRD

SEQ ID NO: 22 - protein, 21 aa, Artificial Sequence
  Synthetic
ATDLTGNWMEEAEHELQMTIM

SEQ ID NO: 23 - protein, 21 aa, Artificial Sequence
  Synthetic
CYGGGQDITIFRYILYQEAIG

SEQ ID NO: 24 - protein, 21 aa, Artificial Sequence
  Synthetic
AMEPTGMAESMPISEVQKMGD

SEQ ID NO: 25 - protein, 21 aa, Artificial Sequence
  Synthetic
FDAEFQDASETCNEIVQCEDM

SEQ ID NO: 26 - protein, 21 aa, Artificial Sequence
  Synthetic
FTCGHITGPSNEPERMQMCMG

SEQ ID NO: 27 - protein, 21 aa, Artificial Sequence
  Synthetic
ITAGPADFSLHPHNAKMNEYY

SEQ ID NO: 28 - protein, 21 aa, Artificial Sequence
  Synthetic
PKEKGGVVKLDNFMNNQWENI

SEQ ID NO: 29 - protein, 21 aa, Artificial Sequence
  Synthetic
GVFFTKRQENESRRKQHQPTA

SEQ ID NO: 30 - protein, 21 aa, Artificial Sequence
  Synthetic
PKMVCMIESERETEEMNCCHV

SEQ ID NO: 31 - protein, 21 aa, Artificial Sequence
  Synthetic
PDTGRWDWSHEGVDEYSRKIR

SEQ ID NO: 32 - protein, 21 aa, Artificial Sequence
  Synthetic
PTQGNCARSLTDLNIKQSELK

SEQ ID NO: 33 - protein, 21 aa, Artificial Sequence
  Synthetic
PGAGCPPASSEEYEGEQEEFG

SEQ ID NO: 34 - protein, 21 aa, Artificial Sequence
  Synthetic
PHEVDGQGSSCWDEEWSTEGT

SEQ ID NO: 35 - protein, 21 aa, Artificial Sequence
  Synthetic
PTAGTGDWSSEEPEEEQEETG

SEQ ID NO: 36 - protein, 21 aa, Artificial Sequence
  Synthetic
PDLINADLSTELQALAAEKTG

SEQ ID NO: 37 - protein, 21 aa, Artificial Sequence
  Synthetic
PSFGLGDINSGNPKEEVEWWA

SEQ ID NO: 38 - protein, 21 aa, Artificial Sequence
  Synthetic
PIAPLRDSSIIEDMEEYEAHG

SEQ ID NO: 39 - protein, 21 aa, Artificial Sequence
  Synthetic
PTQWIPQMEEDENMREQCKAG

SEQ ID NO: 40 - protein, 21 aa, Artificial Sequence
  Synthetic
PIELTRKCVWGSNDEWQEKPK

SEQ ID NO: 41 - protein, 21 aa, Artificial Sequence
  Synthetic
PLLGPVPWSQNEPPDEVEAFQ

SEQ ID NO: 42 - protein, 21 aa, Artificial Sequence
  Synthetic
PTKTCGDPQGIEPQMERYSTV

SEQ ID NO: 43 - protein, 21 aa, Artificial Sequence
  Synthetic
PKYKYTVWMLQEHIFVQNAMD

SEQ ID NO: 44 - protein, 21 aa, Artificial Sequence
  Synthetic
PVAMTRDWKRGDMEMEDAEDG

SEQ ID NO: 45 - protein, 21 aa, Artificial Sequence
  Synthetic
PTFRTFDWIFEEPAADYGFIE

SEQ ID NO: 46 - protein, 21 aa, Artificial Sequence
  Synthetic
PCERMGVWSSEEPEELQNEYS

SEQ ID NO: 47 - protein, 21 aa, Artificial Sequence
  Synthetic
PTRDEVMPSSTDPEEEHWKTS

SEQ ID NO: 48 - protein, 21 aa, Artificial Sequence
  Synthetic
PTMGGGGLSSEEHGWEVEKFM

SEQ ID NO: 49 - protein, 21 aa, Artificial Sequence
  Synthetic
PIECTVDMCCESPEGWQEETG

SEQ ID NO: 50 - protein, 21 aa, Artificial Sequence
  Synthetic
PTEVCGDMSSLEPKEVAEETP

SEQ ID NO: 51 - protein, 21 aa, Artificial Sequence
  Synthetic
PHESDNQRSSESPEEEQRSTG

SEQ ID NO: 52 - protein, 23 aa, Artificial Sequence
  Synthetic
HVRQRDEWVGRFEILDFRYIVKC

SEQ ID NO: 53 - protein, 23 aa, Artificial Sequence
  Synthetic
FVELNTEPEDEAMMLIVEMDYHM

SEQ ID NO: 54 - protein, 23 aa, Artificial Sequence
  Synthetic
QVRRVEYLCGHCEDLQRFAFSIA

SEQ ID NO: 55 - protein, 23 aa, Artificial Sequence
  Synthetic
AMRRSKEGWNEEYRVQRMSKSAM

SEQ ID NO: 56 - protein, 23 aa, Artificial Sequence
  Synthetic
LARPCSWSYCWRERLVRMAKCDP

SEQ ID NO: 57 - protein, 23 aa, Artificial Sequence
  Synthetic
TQTILFEFSGEHHRLIRSSKMVR

SEQ ID NO: 58 - protein, 23 aa, Artificial Sequence
  Synthetic
CDRRVFKEEGCEHTYQDMNRDKP

SEQ ID NO: 59 - protein, 23 aa, Artificial Sequence
  Synthetic
LRLMTNLIPGMTTKLSRAAKIAA

SEQ ID NO: 60 - protein, 23 aa, Artificial Sequence
  Synthetic
LMRTFEESEGERPEDQRTAMSGN

SEQ ID NO: 61 - protein, 23 aa, Artificial Sequence
  Synthetic
LQRLLEEFHWAVNSVQFFNDCAM

SEQ ID NO: 62 - protein, 23 aa, Artificial Sequence
  Synthetic
LQRRLEYAKCERFRLVRDDDYEA

SEQ ID NO: 63 - protein, 23 aa, Artificial Sequence
  Synthetic
WHIPLSENNGCRERLGREAVRCL

SEQ ID NO: 64 - protein, 23 aa, Artificial Sequence
  Synthetic
GQRRLECFCDTRLRGYCMWDSAA

SEQ ID NO: 65 - protein, 23 aa, Artificial Sequence
  Synthetic
LQRHSEQTHGKQEHLKRHADDMC

SEQ ID NO: 66 - protein, 23 aa, Artificial Sequence
  Synthetic
LQRFMEEHSGDRERLPSMNVSKA

SEQ ID NO: 67 - protein, 23 aa, Artificial Sequence
  Synthetic
EQKSLPEQKGERARLQRMRDSAG

SEQ ID NO: 68 - protein, 23 aa, Artificial Sequence
  Synthetic
LQRRMEESEREHEFLQRMADSAA

SEQ ID NO: 69 - protein, 23 aa, Artificial Sequence
  Synthetic
GQERLTEREGEFERQERMHDGRA

SEQ ID NO: 70 - protein, 23 aa, Artificial Sequence
  Synthetic
LARFLEETEGKEENLQRRADSIK

SEQ ID NO: 71 - protein, 23 aa, Artificial Sequence
  Synthetic
LQKDREEFEKTRHRLMKMTDHWA

SEQ ID NO: 72 - protein, 23 aa, Artificial Sequence
  Synthetic
LQNSPEEFAGYRQRCQRMADAAA

SEQ ID NO: 73 - protein, 23 aa, Artificial Sequence
  Synthetic
LQRRLEEWNGERHRLHRMEDIMV

SEQ ID NO: 74 - protein, 23 aa, Artificial Sequence
  Synthetic
WQRRLSEFRREREETWRMAYSAA

SEQ ID NO: 75 - protein, 23 aa, Artificial Sequence
  Synthetic
TQRKLEEFEGERERCSKMAVSVA

SEQ ID NO: 76 - protein, 23 aa, Artificial Sequence
  Synthetic
LQRRLEDFDWSLQRLQRMADSAA

SEQ ID NO: 77 - protein, 23 aa, Artificial Sequence
  Synthetic
LQRRLEEFEWERHRLGMMACSGA

SEQ ID NO: 78 - protein, 23 aa, Artificial Sequence
  Synthetic
LQRRLELFEGEYERLQVMALSAA

SEQ ID NO: 79 - protein, 23 aa, Artificial Sequence
  Synthetic
LIRRLEEFEGERERLQRPADSGA

SEQ ID NO: 80 - protein, 23 aa, Artificial Sequence
  Synthetic
LQKRDEEFEGERERLQRMADTAA

SEQ ID NO: 81 - protein, 23 aa, Artificial Sequence
  Synthetic
LQRRLEEFEGERERLQRMATSDA

SEQ ID NO: 82 - protein, 23 aa, Artificial Sequence
  Synthetic
LARRLEEFEGERERLQRMADSAA

SEQ ID NO: 83 - protein, 23 aa, Artificial Sequence
  Synthetic
NELYGEEAADESENAQRFQDTAH

SEQ ID NO: 84 - protein, 22 aa, Artificial Sequence
  Synthetic
EMSSHIMTQAGVQWPDLGSLEV

SEQ ID NO: 85 - protein, 22 aa, Artificial Sequence
  Synthetic
EMESHSVRQAVVQWWDCGSLEV

SEQ ID NO: 86 - protein, 22 aa, Artificial Sequence
  Synthetic
ENESHSVTIAGEQWPDMGRLEV

SEQ ID NO: 87 - protein, 22 aa, Artificial Sequence
  Synthetic
EMESWSVPQAGVQVPFVGSLED

SEQ ID NO: 88 - protein, 22 aa, Artificial Sequence
  Synthetic
EMETHSDLQALQQWGDHGSLEV

SEQ ID NO: 89 - protein, 22 aa, Artificial Sequence
  Synthetic
EMFSHSQTQLNTQWPDLGTHEV

SEQ ID NO: 90 - protein, 22 aa, Artificial Sequence
  Synthetic
ESESRSVTQDRRVWPFLGSLEP

SEQ ID NO: 91 - protein, 22 aa, Artificial Sequence
  Synthetic
EMASYSVVQAGVQWPSFGDNEF

SEQ ID NO: 92 - protein, 22 aa, Artificial Sequence
  Synthetic
ELESMEVITGGVQWPSWYKLWV

SEQ ID NO: 93 - protein, 22 aa, Artificial Sequence
  Synthetic
EMESHSCMQPGHHWNDRGLNPQ

SEQ ID NO: 94 - protein, 22 aa, Artificial Sequence
  Synthetic
EHFEQSVTQAGHGWSDWGSMEE

SEQ ID NO: 95 - protein, 22 aa, Artificial Sequence
  Synthetic
ESVSTAVTQAGISWPELFEGGR

SEQ ID NO: 96 - protein, 22 aa, Artificial Sequence
  Synthetic
EMYIHSVTCNGDQRRCLGSGDQ

SEQ ID NO: 97 - protein, 22 aa, Artificial Sequence
  Synthetic
EMVSFWVRFIYVMWPVLMSCSY

SEQ ID NO: 98 - protein, 22 aa, Artificial Sequence
  Synthetic
EHEYYSVCQAGVVPWDSGLREV

SEQ ID NO: 99 - protein, 22 aa, Artificial Sequence
  Synthetic
EMESTMVNGRPWQWYKLCHLEI

SEQ ID NO: 100 - protein, 22 aa, Artificial Sequence
  Synthetic
EMESHSVTQAGVQWPDLGSLEV

SEQ ID NO: 101 - protein, 22 aa, Artificial Sequence
  Synthetic
EMSCHYVKQSPNSWDDDASYEV

SEQ ID NO: 102 - protein, 22 aa, Artificial Sequence
  Synthetic
EMGGHSVLQAGVQSENLLCWLS

SEQ ID NO: 103 - protein, 22 aa, Artificial Sequence
  Synthetic
EMEHLLQRFCYPQFLDTNSHID

SEQ ID NO: 104 - protein, 22 aa, Artificial Sequence
  Synthetic
EMDSCDVCEQGLRWKDAGSLTR

SEQ ID NO: 105 - protein, 22 aa, Artificial Sequence
  Synthetic
EFERCHVTAAGEHKCDACSLEN

SEQ ID NO: 106 - protein, 22 aa, Artificial Sequence
  Synthetic
EMCQHKVHDGCVQRCKLAHVRV

SEQ ID NO: 107 - protein, 22 aa, Artificial Sequence
  Synthetic
EFPTTIVGQAMRQIMMTQCLNP

SEQ ID NO: 108 - protein, 22 aa, Artificial Sequence
  Synthetic
EAAVVSVHCICVKLPDRQQKCV

SEQ ID NO: 109 - protein, 22 aa, Artificial Sequence
  Synthetic
ESCKHTQNQSAYTWPSPHWGEV

SEQ ID NO: 110 - protein, 22 aa, Artificial Sequence
  Synthetic
EMNLPSMNLMCVSCLDPCSIGV

SEQ ID NO: 111 - protein, 22 aa, Artificial Sequence
  Synthetic
EMDEHSNTVALMQEFKSWIYTA

SEQ ID NO: 112 - protein, 22 aa, Artificial Sequence
  Synthetic
EMCFHCGRQTGGQMQSQLSLEV

SEQ ID NO: 113 - protein, 22 aa, Artificial Sequence
  Synthetic
ECGYRSVTPFWEQEMNHCSLHG

SEQ ID NO: 114 - protein, 22 aa, Artificial Sequence
  Synthetic
ECISAPQMRVYGQQPFAYNLEE

SEQ ID NO: 115 - protein, 22 aa, Artificial Sequence
  Synthetic
EMTSIIWMCSWAQMKDDMDLSV

SEQ ID NO: 116 - protein, 22 aa, Artificial Sequence
  Synthetic
ENGNCENSKSGQQDSWVQQLEL

SEQ ID NO: 117 - protein, 22 aa, Artificial Sequence
  Synthetic
ETYPVYLVQRQSNIEDLVQVLQ

SEQ ID NO: 118 - protein, 22 aa, Artificial Sequence
  Synthetic
EMPSFRPTMYRVRESVYRAANK

SEQ ID NO: 119 - protein, 22 aa, Artificial Sequence
  Synthetic
EGNNPKLTFAIVQWPHDESTRW

SEQ ID NO: 120 - protein, 22 aa, Artificial Sequence
  Synthetic
EMKQIQVQEFGKCWQRATSHPV

SEQ ID NO: 121 - protein, 22 aa, Artificial Sequence
  Synthetic
EKVPVMATYTGAVWMRGGKEIV

SEQ ID NO: 122 - protein, 22 aa, Artificial Sequence
  Synthetic
ESEFHEVEQAVKKCGFVATLSV

SEQ ID NO: 123 - protein, 22 aa, Artificial Sequence
  Synthetic
NMSAAVTFSASWQTPFRAPFPG

SEQ ID NO: 124 - protein, 22 aa, Artificial Sequence
  Synthetic
EMESHSVTQAGVQMPDLGILEV

SEQ ID NO: 125 - protein, 22 aa, Artificial Sequence
  Synthetic
YLHSCVVNQEWNQLFYLWTLLY

SEQ ID NO: 126 - protein, 22 aa, Artificial Sequence
  Synthetic
TYEAHGVQTEYCRSNISQWDRD

SEQ ID NO: 127 - protein, 22 aa, Artificial Sequence
  Synthetic
LCEHKVTGDEETDASNGTCAEP

SEQ ID NO: 128 - protein, 22 aa, Artificial Sequence
  Synthetic
IMDSFSIKITDWVSPDSDEVKN

SEQ ID NO: 129 - protein, 22 aa, Artificial Sequence
  Synthetic
NQWAWFVPVSSQPSYLEYRKEV

SEQ ID NO: 130 - protein, 22 aa, Artificial Sequence
  Synthetic
EWQQYSVFSLGWMLGVDWGLRR

SEQ ID NO: 131 - protein, 22 aa, Artificial Sequence
  Synthetic
THYQISVCDLKYPIYDFDYNVV

SEQ ID NO: 132 - protein, 22 aa, Artificial Sequence
  Synthetic
FDWSHSGFHAGSQMVQCFLLWG

SEQ ID NO: 133 - protein, 22 aa, Artificial Sequence
  Synthetic
GMLSQAHHEAYVWMMKPYFNLM

SEQ ID NO: 134 - protein, 22 aa, Artificial Sequence
  Synthetic
EMVCWPRTEAIVIKYGVFSREE

SEQ ID NO: 135 - protein, 22 aa, Artificial Sequence
  Synthetic
WADCQDVTQVYVDGYDHKSWIR

SEQ ID NO: 136 - protein, 22 aa, Artificial Sequence
  Synthetic
MTASQSQTPLCKQVWDLCEHKL

SEQ ID NO: 137 - protein, 22 aa, Artificial Sequence
  Synthetic
HHESWSCMPAEVMKTGLAKGAS

SEQ ID NO: 138 - protein, 22 aa, Artificial Sequence
  Synthetic
IMDATCVTNVGYQDHDEQMINV

SEQ ID NO: 139 - protein, 22 aa, Artificial Sequence
  Synthetic
RMECHCTGEMGVRVIFIGGTEE

SEQ ID NO: 140 - protein, 22 aa, Artificial Sequence
  Synthetic
PTTEHTPTDTGYFKHHNASLEH

SEQ ID NO: 141 - protein, 22 aa, Artificial Sequence
  Synthetic
RLQSLYATQQGYRDPWTGHEEV

SEQ ID NO: 142 - protein, 22 aa, Artificial Sequence
  Synthetic
EMESQKMFDCRMQGPVVVGEEV

SEQ ID NO: 143 - protein, 22 aa, Artificial Sequence
  Synthetic
PGESHSQFRNYDLVRYTILQEV

SEQ ID NO: 144 - protein, 22 aa, Artificial Sequence
  Synthetic
CMMNHSLTAIILQGMRKHELDW

SEQ ID NO: 145 - protein, 22 aa, Artificial Sequence
  Synthetic
EMFCQSESQACVAIPQTGCHLV

SEQ ID NO: 146 - protein, 22 aa, Artificial Sequence
  Synthetic
TMHKSNVNKVSVPWCDRGSLTV

SEQ ID NO: 147 - protein, 22 aa, Artificial Sequence
  Synthetic
EMHCHPYYGARVQYPKLYSDEV

SEQ ID NO: 148 - protein, 22 aa, Artificial Sequence
  Synthetic
EMEDTWETQLPVRWHDLPDYMY

SEQ ID NO: 149 - protein, 22 aa, Artificial Sequence
  Synthetic
SMECHSCCQKLMMWRALRSLEA

SEQ ID NO: 150 - protein, 22 aa, Artificial Sequence
  Synthetic
DMTSMMTRQAFFQWPLPGWADV

SEQ ID NO: 151 - protein, 22 aa, Artificial Sequence
  Synthetic
YKEYHSVYSPTVVEPPLVSLEW

SEQ ID NO: 152 - protein, 22 aa, Artificial Sequence
  Synthetic
VCTSQQVLIAVLQMSADIQLEV

SEQ ID NO: 153 - protein, 22 aa, Artificial Sequence
  Synthetic
IQLSPSVTQAGFVMPDLGSREV

SEQ ID NO: 154 - protein, 22 aa, Artificial Sequence
  Synthetic
EMEHHSWPLFDVQWHHLNPLEG

SEQ ID NO: 155 - protein, 22 aa, Artificial Sequence
  Synthetic
DMLEHIITQADVQIPDVGSLEF

SEQ ID NO: 156 - protein, 22 aa, Artificial Sequence
  Synthetic
EGLSHGFTQAIIQWPDLGSMWV

SEQ ID NO: 157 - protein, 22 aa, Artificial Sequence
  Synthetic
HMESHSVHQQGVQRPRLGWEEC

SEQ ID NO: 158 - protein, 22 aa, Artificial Sequence
  Synthetic
MMESHSVASAGVQWKKNPTLIV

SEQ ID NO: 159 - protein, 22 aa, Artificial Sequence
  Synthetic
EMESHSVAQHGVQWSGNSSMGV

SEQ ID NO: 160 - protein, 23 aa, Artificial Sequence
  Synthetic
LQRHLEEFEGERERLQRMADSAA

SEQ ID NO: 161 - protein, 23 aa, Artificial Sequence
  Synthetic
LQRRLEEFEGERERLQRVADSKA

SEQ ID NO: 162 - protein, 23 aa, Artificial Sequence
  Synthetic
LDRRLEEFEGERGRLQRMADSNA

SEQ ID NO: 163 - protein, 23 aa, Artificial Sequence
  Synthetic
LQRRCEEFHGEYERLQPMADSAA

SEQ ID NO: 164 - protein, 23 aa, Artificial Sequence
  Synthetic
LERRFEEFWGERVRLQRMADSAL

SEQ ID NO: 165 - protein, 23 aa, Artificial Sequence
  Synthetic
LQRRLEMFEHGGERLQRYADFAA

SEQ ID NO: 166 - protein, 23 aa, Artificial Sequence
  Synthetic
LPRRTEEPVGERERLQRCMDSAA

SEQ ID NO: 167 - protein, 23 aa, Artificial Sequence
  Synthetic
LQKRDAGFEGERERFQRMASSAA

SEQ ID NO: 168 - protein, 23 aa, Artificial Sequence
  Synthetic
LQRRGEEQEGERERLQRVSDSSS

SEQ ID NO: 169 - protein, 23 aa, Artificial Sequence
  Synthetic
LQRRGHEFEMERRRLQRMAYHAF

SEQ ID NO: 170 - protein, 23 aa, Artificial Sequence
  Synthetic
LLNELQVFEFERFHLQRMADSIA

SEQ ID NO: 171 - protein, 23 aa, Artificial Sequence
  Synthetic
LQGRVNEFNGRRERLDRMIRFAG

SEQ ID NO: 172 - protein, 23 aa, Artificial Sequence
  Synthetic
LQRYQEEVMNEDERVQEMEDSAH

SEQ ID NO: 173 - protein, 23 aa, Artificial Sequence
  Synthetic
LERVLEEFYTERHKAPKMAHTFA

SEQ ID NO: 174 - protein, 23 aa, Artificial Sequence
  Synthetic
LQRCLEEFEDTRCRLGHMPISDA

SEQ ID NO: 175 - protein, 23 aa, Artificial Sequence
  Synthetic
LIAHLEKFCYERTILMDMIKSAA

SEQ ID NO: 176 - protein, 23 aa, Artificial Sequence
  Synthetic
LQYCLERFRGLRERWGRSKDSYH

SEQ ID NO: 177 - protein, 23 aa, Artificial Sequence
  Synthetic
LQRTLMMFEGNRKLMSVMAMYTA

SEQ ID NO: 178 - protein, 23 aa, Artificial Sequence
  Synthetic
LCRFLKEGKGDREVVVRGAMSKQ

SEQ ID NO: 179 - protein, 23 aa, Artificial Sequence
  Synthetic
LARLLEEGEWVILRLWELRTGFA

SEQ ID NO: 180 - protein, 23 aa, Artificial Sequence
  Synthetic
LFRYLKEQNAEPECGVSFYTHAA

SEQ ID NO: 181 - protein, 23 aa, Artificial Sequence
  Synthetic
LQHRLAEFELYIEERQDPAKRWC

SEQ ID NO: 182 - protein, 23 aa, Artificial Sequence
  Synthetic
LPRIIAEFEGLREMAGRMANSRP

SEQ ID NO: 183 - protein, 23 aa, Artificial Sequence
  Synthetic
LTRFYHYFDGMGYRATWYQYGMA

SEQ ID NO: 184 - protein, 23 aa, Artificial Sequence
  Synthetic
LERRKECTQGDRFPYLMMADQVC

SEQ ID NO: 185 - protein, 23 aa, Artificial Sequence
  Synthetic
LRRHLTEIPGHNAECQDFKWWKW

SEQ ID NO: 186 - protein, 23 aa, Artificial Sequence
  Synthetic
LQMRLEWKKHMRNKLVIPDPECA

SEQ ID NO: 187 - protein, 23 aa, Artificial Sequence
  Synthetic
LHTNCMVEEQIAEPLYAKADYNG

SEQ ID NO: 188 - protein, 23 aa, Artificial Sequence
  Synthetic
LFRSWWILDTEDEASGSVTPAMT

SEQ ID NO: 189 - protein, 23 aa, Artificial Sequence
  Synthetic
LFRMTEGGYDCPWHLARTGDSDG

SEQ ID NO: 190 - protein, 23 aa, Artificial Sequence
  Synthetic
LIRWRYPDEGMRTQAAAMAFQGI

SEQ ID NO: 191 - protein, 23 aa, Artificial Sequence
  Synthetic
LNTLSVMDEVKGYNMQWEATSAA

SEQ ID NO: 192 - protein, 23 aa, Artificial Sequence
  Synthetic
QEIRLSVPEMTEVRMSRCTDMAD

SEQ ID NO: 193 - protein, 23 aa, Artificial Sequence
  Synthetic
CWRPSWECEGLHHGAVRSDHTWT

SEQ ID NO: 194 - protein, 23 aa, Artificial Sequence
  Synthetic
KQRNGAEFEGFPEQLWRPADYVV

SEQ ID NO: 195 - protein, 23 aa, Artificial Sequence
  Synthetic
MSPRLWETLVELETTQRCSLTRH

SEQ ID NO: 196 - protein, 23 aa, Artificial Sequence
  Synthetic
EAGRTFTYKYSLWRGYTFSDSAN

SEQ ID NO: 197 - protein, 23 aa, Artificial Sequence
  Synthetic
LQRRLEEFEGERERLQRMADSAA

SEQ ID NO: 198 - protein, 23 aa, Artificial Sequence
  Synthetic
SNGLEEEPFRVRGHTRECFDIAA

SEQ ID NO: 199 - protein, 23 aa, Artificial Sequence
  Synthetic
DGCRLDEVEGEFRKISPWKDVLA

SEQ ID NO: 200 - protein, 23 aa, Artificial Sequence
  Synthetic
LFERLAMFEHWYHDKFKAKDSDA

SEQ ID NO: 201 - protein, 23 aa, Artificial Sequence
  Synthetic
TPRRHNQFYHQKERLQDNGDFMA

SEQ ID NO: 202 - protein, 23 aa, Artificial Sequence
  Synthetic
LQPLLPIWEGERMRELRMYDSEA

SEQ ID NO: 203 - protein, 23 aa, Artificial Sequence
  Synthetic
LQLNLEGFCGECPRPVRMAKSAY

SEQ ID NO: 204 - protein, 23 aa, Artificial Sequence
  Synthetic
HQRRLSFFEVRILNKHRYAHSCA

SEQ ID NO: 205 - protein, 23 aa, Artificial Sequence
  Synthetic
LSRRLTEQEGTFERSQKFFDMQA

SEQ ID NO: 206 - protein, 23 aa, Artificial Sequence
  Synthetic
SQMRRKHFIHEREYLYRMLDSIQ

SEQ ID NO: 207 - protein, 23 aa, Artificial Sequence
  Synthetic
LQRHMEDEEDKRDQVQRMADTAA

SEQ ID NO: 208 - protein, 23 aa, Artificial Sequence
  Synthetic
KYRNHEEFEGENIRYQVWADHKH

SEQ ID NO: 209 - protein, 23 aa, Artificial Sequence
  Synthetic
LCRGLEREEGEREEFHRMATQAI

SEQ ID NO: 210 - protein, 23 aa, Artificial Sequence
  Synthetic
YMRKLEEWHGEIRRHQPLADFAA

SEQ ID NO: 211 - protein, 23 aa, Artificial Sequence
  Synthetic
TLVGREEFEGEGQMLQRMADSAD

SEQ ID NO: 212 - protein, 23 aa, Artificial Sequence
  Synthetic
LQRRFENFEGGRERLIRMADFAA

SEQ ID NO: 213 - protein, 23 aa, Artificial Sequence
  Synthetic
FVRFREEFEGWRERLQRMAPSAA

SEQ ID NO: 214 - protein, 23 aa, Artificial Sequence
  Synthetic
LQRRVAHFEGERARLQRMADSAA

SEQ ID NO: 215 - protein, 23 aa, Artificial Sequence
  Synthetic
KQRRLEEFEGECEQLQRIAPSAK

SEQ ID NO: 216 - protein, 23 aa, Artificial Sequence
  Synthetic
LQRRLEEFEGERYALSRMAASEA

SEQ ID NO: 217 - protein, 23 aa, Artificial Sequence
  Synthetic
MQRVLAEFEGERQRLQRMADSAA

SEQ ID NO: 218 - protein, 23 aa, Artificial Sequence
  Synthetic
LQRILEEFEGERERLQREADSAA

SEQ ID NO: 219 - protein, 23 aa, Artificial Sequence
  Synthetic
LQRRLEEFEDERERLQRMAQSAA

SEQ ID NO: 220 - protein, 23 aa, Artificial Sequence
  Synthetic
LQRRLEEFFGERERLQRMADSAA

SEQ ID NO: 221 - protein, 22 aa, Artificial Sequence
  Synthetic
RKWLEEQLKQYRVKVQQERSSQ

SEQ ID NO: 222 - protein, 22 aa, Artificial Sequence
  Synthetic
RAWLEKQLKQYRVKRQQERSSQ

SEQ ID NO: 223 - protein, 22 aa, Artificial Sequence
  Synthetic
RSWLEEQLKQMRAKRQQERSSQ

SEQ ID NO: 224 - protein, 22 aa, Artificial Sequence
  Synthetic
RKWSEEQLKQYRVKRQQEWSSR

SEQ ID NO: 225 - protein, 22 aa, Artificial Sequence
  Synthetic
RKALEQQLKQYRVKRQQSRSSQ

SEQ ID NO: 226 - protein, 22 aa, Artificial Sequence
  Synthetic
RKWLEEWLKSYRKHRQQERSAQ

SEQ ID NO: 227 - protein, 22 aa, Artificial Sequence
  Synthetic
RKWSEEQLKGYYSKRQQERMSQ

SEQ ID NO: 228 - protein, 22 aa, Artificial Sequence
  Synthetic
RRFLEKELKQYRHGRQQERSSR

SEQ ID NO: 229 - protein, 22 aa, Artificial Sequence
  Synthetic
RDWLETQLKQCIVKRNQNSSMQ

SEQ ID NO: 230 - protein, 22 aa, Artificial Sequence
  Synthetic
RKWLEGVLKGYLVKSQLESSSG

SEQ ID NO: 231 - protein, 22 aa, Artificial Sequence
  Synthetic
RFWLKEQLKQYRVKGTVELSRQ

SEQ ID NO: 232 - protein, 22 aa, Artificial Sequence
  Synthetic
RDWHIEELKQFRVKIQGTRSSN

SEQ ID NO: 233 - protein, 22 aa, Artificial Sequence
  Synthetic
RKWCEEQLIQFRVFEQGSRYRQ

SEQ ID NO: 234 - protein, 22 aa, Artificial Sequence
  Synthetic
RKWLEMALKHFKMSRQSEISSQ

SEQ ID NO: 235 - protein, 22 aa, Artificial Sequence
  Synthetic
RKNLEDQMKGLRVFPGDCGSAQ

SEQ ID NO: 236 - protein, 22 aa, Artificial Sequence
  Synthetic
RKWLEEQLKQYRVKRQQERSSQ

SEQ ID NO: 237 - protein, 22 aa, Artificial Sequence
  Synthetic
RKWRDYCLKQYDFDISLERLVR

SEQ ID NO: 238 - protein, 22 aa, Artificial Sequence
  Synthetic
RKWKGMHDSLYRVKYQGEMNSG

SEQ ID NO: 239 - protein, 22 aa, Artificial Sequence
  Synthetic
RIWLYWQIKQWFVLGHQKRSSE

SEQ ID NO: 240 - protein, 22 aa, Artificial Sequence
  Synthetic
RKIIEEINMLYMKKRIDEHSSQ

SEQ ID NO: 241 - protein, 22 aa, Artificial Sequence
  Synthetic
RKWVEEMLRDGFVKNARWFGPD

SEQ ID NO: 242 - protein, 22 aa, Artificial Sequence
  Synthetic
RVWEALRRKGERYSAQQHSSSE

SEQ ID NO: 243 - protein, 22 aa, Artificial Sequence
  Synthetic
RILAIERKGRYDDKTQQQDYFT

SEQ ID NO: 244 - protein, 22 aa, Artificial Sequence
  Synthetic
RKHRSMCLPQNRCLMGSEDIQQ

SEQ ID NO: 245 - protein, 22 aa, Artificial Sequence
  Synthetic
RHYCFEIGKQCQHKMANASCPM

SEQ ID NO: 246 - protein, 22 aa, Artificial Sequence
  Synthetic
RSDIEPHFSAYDFTDKDNCSHQ

SEQ ID NO: 247 - protein, 22 aa, Artificial Sequence
  Synthetic
RECLERFMVDYDMCDHQARSGQ

SEQ ID NO: 248 - protein, 22 aa, Artificial Sequence
  Synthetic
RKYLIEILKLFRPWAQQCRHHL

SEQ ID NO: 249 - protein, 22 aa, Artificial Sequence
  Synthetic
RIYTLDCIKQQRQFTQWGMSQA

SEQ ID NO: 250 - protein, 22 aa, Artificial Sequence
  Synthetic
RGRDCCAINQKHNNFNVEPCSS

SEQ ID NO: 251 - protein, 22 aa, Artificial Sequence
  Synthetic
RELLEAQVNQHDYKYLQQQSFQ

SEQ ID NO: 252 - protein, 22 aa, Artificial Sequence
  Synthetic
RKELRSWSAWYKPEEEQSRSCI

SEQ ID NO: 253 - protein, 22 aa, Artificial Sequence
  Synthetic
RGQQMEHLRRCLPIRQAEYCDA

SEQ ID NO: 254 - protein, 22 aa, Artificial Sequence
  Synthetic
HIKCEPMPKQCMVKASMRRYSN

SEQ ID NO: 255 - protein, 22 aa, Artificial Sequence
  Synthetic
DFGVWEWGKQTPYKRQQWPLDD

SEQ ID NO: 256 - protein, 22 aa, Artificial Sequence
  Synthetic
TRWSEGQAKFWLNQIVQEWSST

SEQ ID NO: 257 - protein, 22 aa, Artificial Sequence
  Synthetic
GKWAGITGDPERVESQADFFVP

SEQ ID NO: 258 - protein, 22 aa, Artificial Sequence
  Synthetic
RKTFEGWLYPYNTKIFEERSQG

SEQ ID NO: 259 - protein, 22 aa, Artificial Sequence
  Synthetic
NKWLMMIGYNYDLCEYHECNDR

SEQ ID NO: 260 - protein, 22 aa, Artificial Sequence
  Synthetic
QKWWEEYLTQEDVGRWQKASYW

SEQ ID NO: 261 - protein, 22 aa, Artificial Sequence
  Synthetic
QKWSEYQHHVYEGPRYEMFFQR

SEQ ID NO: 262 - protein, 22 aa, Artificial Sequence
  Synthetic
FFMLEVKLETMRAGHQPEDETW

SEQ ID NO: 263 - protein, 22 aa, Artificial Sequence
  Synthetic
MKVLGEEVFQYRVINGQQCSYT

SEQ ID NO: 264 - protein, 22 aa, Artificial Sequence
  Synthetic
GTWGPEQGKMYRGKFRQNASYQ

SEQ ID NO: 265 - protein, 22 aa, Artificial Sequence
  Synthetic
MKWDIAQLKTKCVDHGTNPCSN

SEQ ID NO: 266 - protein, 22 aa, Artificial Sequence
  Synthetic
SAWLTEQLVQYRVKGQAEKSSQ

SEQ ID NO: 267 - protein, 22 aa, Artificial Sequence
  Synthetic
LCWHEEYLKQCRVKRQICNSLP

SEQ ID NO: 268 - protein, 22 aa, Artificial Sequence
  Synthetic
RCWGEEQDKQLAVSRQDEIMQQ

SEQ ID NO: 269 - protein, 22 aa, Artificial Sequence
  Synthetic
EKQLEKETHQLSVKRQQPRLNF

SEQ ID NO: 270 - protein, 22 aa, Artificial Sequence
  Synthetic
EKKEENQLKMNPAKRQPERSVR

SEQ ID NO: 271 - protein, 22 aa, Artificial Sequence
  Synthetic
HKTTEEWLKQYWVKVQQERRSQ

SEQ ID NO: 272 - protein, 22 aa, Artificial Sequence
  Synthetic
TDWFNEQLKVYREKRQQERTHQ

SEQ ID NO: 273 - protein, 22 aa, Artificial Sequence
  Synthetic
RKQAEDQLAQYRVKRWQERSAI

SEQ ID NO: 274 - protein, 22 aa, Artificial Sequence
  Synthetic
VKFPEEQQGMYRWKRQFERSSQ

SEQ ID NO: 275 - protein, 22 aa, Artificial Sequence
  Synthetic
GKWLEEDFKITRVKRTQERSSQ

SEQ ID NO: 276 - protein, 22 aa, Artificial Sequence
  Synthetic
RKWLEEQQQLYRVTRGQGRSSQ

SEQ ID NO: 277 - protein, 22 aa, Artificial Sequence
  Synthetic
RKWLEEQGQGYRVERQQEWSSQ

SEQ ID NO: 278 - protein, 22 aa, Artificial Sequence
  Synthetic
RKWLEEQLLWHEVKRQQERSSQ

SEQ ID NO: 279 - protein, 22 aa, Artificial Sequence
  Synthetic
RKWLEEQLKSYKVKRQQERSHQ

SEQ ID NO: 280 - protein, 22 aa, Artificial Sequence
  Synthetic
RKWLELQLKQYRVKRQQERYSQ

SEQ ID NO: 281 - protein, 22 aa, Artificial Sequence
  Synthetic
RKWLEEQLKQYRVHRQQERSSQ

SEQ ID NO: 282 - protein, 22 aa, Artificial Sequence
  Synthetic
RDQRRKMVLENPRMRNSEISKQ

SEQ ID NO: 283 - protein, 22 aa, Artificial Sequence
  Synthetic
RDQRRQMALENIRMRNSEISKQ

SEQ ID NO: 284 - protein, 22 aa, Artificial Sequence
  Synthetic
RDQRRRMALENPRMRWSNISKQ

SEQ ID NO: 285 - protein, 22 aa, Artificial Sequence
  Synthetic
RDQRRKMRLENPRMGNSEISWY

SEQ ID NO: 286 - protein, 22 aa, Artificial Sequence
  Synthetic
RDQRRKVLREPPRMRNSEISKQ

SEQ ID NO: 287 - protein, 22 aa, Artificial Sequence
  Synthetic
RDQRSKFCLECPRMRNSCISKR

SEQ ID NO: 288 - protein, 22 aa, Artificial Sequence
  Synthetic
RDQRRLMAFENPRMKNHELSKA

SEQ ID NO: 289 - protein, 22 aa, Artificial Sequence
  Synthetic
RMDRIKMAKEHPQSRNSEISKQ

SEQ ID NO: 290 - protein, 22 aa, Artificial Sequence
  Synthetic
RDQRGKMTEENPCMRNFEISKW

SEQ ID NO: 291 - protein, 22 aa, Artificial Sequence
  Synthetic
RDQYRKMRLENIFRRMSEIQKI

SEQ ID NO: 292 - protein, 22 aa, Artificial Sequence
  Synthetic
RNQRRGQAPENYYMRNSDISDQ

SEQ ID NO: 293 - protein, 22 aa, Artificial Sequence
  Synthetic
RPQRSEQSLENPRMRYDEGSKW

SEQ ID NO: 294 - protein, 22 aa, Artificial Sequence
  Synthetic
RDTRKTDALEHPRMRNREGSRQ

SEQ ID NO: 295 - protein, 22 aa, Artificial Sequence
  Synthetic
RNHRRKMRLLNPRMRMSEIQGA

SEQ ID NO: 296 - protein, 22 aa, Artificial Sequence
  Synthetic
RDYRTKMSLIVPRQRYYEIWKM

SEQ ID NO: 297 - protein, 22 aa, Artificial Sequence
  Synthetic
RMKRRFCDVENMRMRGYLISEI

SEQ ID NO: 298 - protein, 22 aa, Artificial Sequence
  Synthetic
RIMRYSGAHKNPHMWRSEYSNQ

SEQ ID NO: 299 - protein, 22 aa, Artificial Sequence
  Synthetic
RGQMRRTWCENWQTRSSRIRPQ

SEQ ID NO: 300 - protein, 22 aa, Artificial Sequence
  Synthetic
RDNRRMMALTYPPGRNKWKLDK

SEQ ID NO: 301 - protein, 22 aa, Artificial Sequence
  Synthetic
RPQHDEMLIINPIMRDSNLTKQ

SEQ ID NO: 302 - protein, 22 aa, Artificial Sequence
  Synthetic
RDKRRKMPNVNEQCQDAQISLL

SEQ ID NO: 303 - protein, 22 aa, Artificial Sequence
  Synthetic
RDQKAEQDNENPKMRMVECHGQ

SEQ ID NO: 304 - protein, 22 aa, Artificial Sequence
  Synthetic
RDEGDKVLDLCNRMMNSRKMIQ

SEQ ID NO: 305 - protein, 22 aa, Artificial Sequence
  Synthetic
RDVRMKMTRPHCRRRCGEASVV

SEQ ID NO: 306 - protein, 22 aa, Artificial Sequence
  Synthetic
RKQHYSPSLENKCARKTCQSKI

SEQ ID NO: 307 - protein, 22 aa, Artificial Sequence
  Synthetic
RLIRCLFCLEMPRIYIPEIHWL

SEQ ID NO: 308 - protein, 22 aa, Artificial Sequence
  Synthetic
RLIRCLFCLEMPRIYIPEIHWL

SEQ ID NO: 309 - protein, 22 aa, Artificial Sequence
  Synthetic
RCNWRKCNEEPPNYMHSMFGCQ

SEQ ID NO: 310 - protein, 22 aa, Artificial Sequence
  Synthetic
RDQRRKMALENPRMRNSEISKQ

SEQ ID NO: 311 - protein, 22 aa, Artificial Sequence
  Synthetic
RQCRKRWRDNNNPMACSYIRKQ

SEQ ID NO: 312 - protein, 22 aa, Artificial Sequence
  Synthetic
REQKRPVDVETDFTLRTPHKKK

SEQ ID NO: 313 - protein, 22 aa, Artificial Sequence
  Synthetic
RHQKVPDAIENPRMRWNGWDTA

SEQ ID NO: 314 - protein, 22 aa, Artificial Sequence
  Synthetic
RIFRRKVYLERNSYRGDWIWTR

SEQ ID NO: 315 - protein, 22 aa, Artificial Sequence
  Synthetic
RKQGRPMDCMYPNMHRGYHMHP

SEQ ID NO: 316 - protein, 22 aa, Artificial Sequence
  Synthetic
RYKSPMTLINRGQIRDTPCSDR

SEQ ID NO: 317 - protein, 22 aa, Artificial Sequence
  Synthetic
CRQDDIKLLHEGEMEKGRLWKH

SEQ ID NO: 318 - protein, 22 aa, Artificial Sequence
  Synthetic
RDARCWWTTHGYHHGNCEWYLK

SEQ ID NO: 319 - protein, 22 aa, Artificial Sequence
  Synthetic
DDQDRKMYTDAPINRLRKALKQ

SEQ ID NO: 320 - protein, 22 aa, Artificial Sequence
  Synthetic
CRADSWKAKEYPENRPSEIPIQ

SEQ ID NO: 321 - protein, 22 aa, Artificial Sequence
  Synthetic
RDAYRPVAVHNCMMRMGMIWAE

SEQ ID NO: 322 - protein, 22 aa, Artificial Sequence
  Synthetic
HTQRNFCFIENTQYWNLEDSWT

SEQ ID NO: 323 - protein, 22 aa, Artificial Sequence
  Synthetic
HDQRRDHRVGVPRMERGTPAKK

SEQ ID NO: 324 - protein, 22 aa, Artificial Sequence
  Synthetic
RMFCRHVAYDLPRMRFSYISVQ

SEQ ID NO: 325 - protein, 22 aa, Artificial Sequence
  Synthetic
RDMIREQAQEKPRLRLSHRWKQ

SEQ ID NO: 326 - protein, 22 aa, Artificial Sequence
  Synthetic
YDPRINYPLEHIRMSNPEIAKE

SEQ ID NO: 327 - protein, 22 aa, Artificial Sequence
  Synthetic
EDARVNMALELARPRKSFRMSH

SEQ ID NO: 328 - protein, 22 aa, Artificial Sequence
  Synthetic
FKQRRFMAMEEPLSRLSERHSC

SEQ ID NO: 329 - protein, 22 aa, Artificial Sequence
  Synthetic
VTERRKMALKFPPDDAVQISHQ

SEQ ID NO: 330 - protein, 22 aa, Artificial Sequence
  Synthetic
RPQRRYTGEYNVRFRCEEISHA

SEQ ID NO: 331 - protein, 22 aa, Artificial Sequence
  Synthetic
TKQTFKMAVGNGTSRNVEINKA

SEQ ID NO: 332 - protein, 22 aa, Artificial Sequence
  Synthetic
RDQRRVVALVKANHYASLIAKG

SEQ ID NO: 333 - protein, 22 aa, Artificial Sequence
  Synthetic
RKQKNKMAKENPRMRHSETNKP

SEQ ID NO: 334 - protein, 22 aa, Artificial Sequence
  Synthetic
RDQRQPMNLWPAAMRNSRIYRQ

SEQ ID NO: 335 - protein, 22 aa, Artificial Sequence
  Synthetic
RDQQRKKALENPRHRNSAKHTK

SEQ ID NO: 336 - protein, 22 aa, Artificial Sequence
  Synthetic
NDQRRKFAWGPPRMRNEPISSE

SEQ ID NO: 337 - protein, 22 aa, Artificial Sequence
  Synthetic
RRQRRKMAWEQPRMQTSVIWRD

SEQ ID NO: 338 - protein, 22 aa, Artificial Sequence
  Synthetic
SDQRMHMALENARWRNSYIWKQ

SEQ ID NO: 339 - protein, 22 aa, Artificial Sequence
  Synthetic
RDQGRKMCLENPRQRNKPIKKQ

SEQ ID NO: 340 - protein, 22 aa, Artificial Sequence
  Synthetic
RDQRMLMTLENPRMRNRAYSWQ

SEQ ID NO: 341 - protein, 22 aa, Artificial Sequence
  Synthetic
RDQTRAAALENPRMRYSEISKY

SEQ ID NO: 342 - protein, 22 aa, Artificial Sequence
  Synthetic
RDQRRKNALEPPRMRHSECSKQ

SEQ ID NO: 343 - protein, 22 aa, Artificial Sequence
  Synthetic
RDQRRKMGLEQPRMRNSEIMKQ

SEQ ID NO: 344 - protein, 22 aa, Artificial Sequence
  Synthetic
RDQRRKMALENPKMSNSEISMQ

SEQ ID NO: 345 - protein, 22 aa, Artificial Sequence
  Synthetic
RDQRRKMALENPKMSNSEISMQ

SEQ ID NO: 346 - protein, 22 aa, Artificial Sequence
  Synthetic
RDQRRKMAKENPRMRNSEISKQ

SEQ ID NO: 347 - protein, 22 aa, Artificial Sequence
  Synthetic
EMESHSVTQAGVQWPDLGSLEV

SEQ ID NO: 348 - DNA, 348 bp, Artificial Sequence
  Synthetic
gaagtgaaggttgaggagtctggaggaggcttggtgcaacctgggggatccatgaaaatctcctgtgttgcctctggattcactttcaagaactactggatgaactgggtccgccagtctccagagaaggggcttgagtgggttgctgaaattagatcgaaatctaataataatgaaaaacattatgcggagtctgtgaaagggaggttcaccatctcaagagatgattttaaaagtagtgtgtacctgcaaatgaacaacttaagaactgaagacactggcatttattactgtacgggggggacctttgactactggggccaaggcaccactctcacagtctcctca

SEQ ID NO: 349 - DNA, 339 bp, Artificial Sequence
  Synthetic
gatgttgtggtgacccagactccactctccctgcctgtcagtcttggagatcaagcctccatctcttgcagatctagtcacagccttgtacacagtgatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaactacccatgttcctccgtacacgttcgggggggggacccagctggaaataaaa

SEQ ID NO: 350 - protein, 116 aa, Artificial Sequence
  Synthetic
EVKVEESGGGLVQPGGSMKISCVASGFTFKNYWMNWVRQSPEKGLEWVAEIRSKSNNNEKHYAESVKGRFTISRDDFKSSVYLQMNNLRTEDTGIYYCTGGTFDYWGQGTTLTVSS

SEQ ID NO: 351 - protein, 113 aa, Artificial Sequence
  Synthetic
DVVVTQTPLSLPVSLGDQASISCRSSHSLVHSDGNTYLHWYLQKPGQSPKLLIYKVSNRFSGVPDRFSGSGSGTDFTLKISRVEAEDLGVYFCSQTTHVPPYTFGGGTQLEIK

SEQ ID NO: 352 - protein, 221 aa, Artificial Sequence
  Synthetic
DBYIDKVVVPAVLALVLVAEAAAAVVEVVTATAEDLVEVMLIFLLGRAFCFSFFFFFEMESHSVTQAGVQWPDLGSLEVTLLPQPPKVGLQVGGNMPSSFFSIFNRDGVSPCWPGWSLPPDLMIHTPWPPEVLGLQAATVPGLGSLFFLRVLFFKAFIGEIFLRDTKSNSRFLLLVLCSTEKKGINELNFSLNIFLDRWLWRLLQWIWRKLLPGGLVGQLN

SEQ ID NO: 353 - protein, 10 aa, Artificial Sequence
  Synthetic
SFFFFEMESH

SEQ ID NO: 354 - protein, 9 aa, Artificial Sequence
  Synthetic
FSIFNRDGV

SEQ ID NO: 355 - protein, 9 aa, Artificial Sequence
  Synthetic
SLNIFLDRW

SEQ ID NO: 356 - DNA, 22 bp, Artificial Sequence
  AO
gttgcactttcacggacgcagc

SEQ ID NO: 357 - DNA, 22 bp, Artificial Sequence
  AO
ctagcccattgctcgccatagc

SEQ ID NO: 358 - DNA, 22 bp, Artificial Sequence
  AO
aatccaccatacctcatggacc

SEQ ID NO: 359 - DNA, 20 bp, Artificial Sequence
  AO
tttctcctgtatcctcctgc

SEQ ID NO: 360 - protein, 11 aa, Artificial Sequence
  AO
HSLVHSDGNTY

SEQ ID NO: 361 - protein, 3 aa, Artificial Sequence
  AO
KVS

SEQ ID NO: 362 - protein, 10 aa, Artificial Sequence
  AO
SQTTHVPPYT

SEQ ID NO: 363 - protein, 8 aa, Artificial Sequence
  AO
GFTFKNYW

SEQ ID NO: 364 - protein, 10 aa, Artificial Sequence
  AO
IRSKSNNNEK

SEQ ID NO: 365 - protein, 7 aa, Artificial Sequence
  AO
TGGTFDY

SEQ ID NO: 366 - DNA, 26 bp, Artificial Sequence
  AO
caccctcctcaagcatgtagtaataa

SEQ ID NO: 367 - DNA, 27 bp, Artificial Sequence
  AO
gcttaccttgttacgacttgtctcttc

SEQ ID NO: 368 - DNA, 23 bp, Artificial Sequence
  AO
ctatattcaattacacaaccatg

SEQ ID NO: 369 - DNA, 21 bp, Artificial Sequence
  AO
tgccacacaaggcatattctg

SEQ ID NO: 370 - DNA, 24 bp, Artificial Sequence
  AO
caactccaaacgtgctctacttca

SEQ ID NO: 371 - DNA, 22 bp, Artificial Sequence
  AO
aatccaccatacctcatggacc

SEQ ID NO: 372 - DNA, 20 bp, Artificial Sequence
  AO
gcaggaggatacaggagaaa

SEQ ID NO: 373 - DNA, 18 bp, Artificial Sequence
  AO
actttcttgggagagcac

SEQ ID NO: 374 - protein, 227 aa, Artificial Sequence
  AO
DVVVTQTPGALPVSLGDQASISCRSSGSLVHSDGNTYLHWYLQKPQSPKLLIYKVSNRFSGVKARFSGSGSGTDFTLKISSLTTEDLGVYFCSQTTHVPPFAFGGGTQLEIKEVKVEESPATLVQPGGSMKISCVASGFVSNNYWMNWVRQSPEKGLEWVAEIRSQSNNNEKHYAESVKGRFTISRDDFKSDFTLQMNNLRTEDTGIYYCTGGRPDTWGQGTTLELS

SEQ ID NO: 375 - protein, 242 aa, Artificial Sequence
  AO
DVVMTQTPLSLPVSLGDQASISCRSSQSLVHSNGNTYLYWYLQKPGQSPKPLIYRVSNRKSGVPDRFSGSGSGTDTLKISRVEAEDLGVYFCFQGTHVPYTFGGGTRLEIKGGGGSGGGGSGGGGSQIQLQQSGPELVRPGASVKISCKASGYTFTDYYIHWVKQRPGEGLEWIGWIYPGSGNTKYNEKFKGKATLTVDTSSSTAYMQLSSLTSEDSAVYFCARGGKFAMDYWGQGTSVTVS

SEQ ID NO: 376 - protein, 217 aa, Artificial Sequence
  AO
IDKVVVPAVLALVLVAEAAAAVVEVVTATAEDLVEVMLIFLLGRAFCFSFFFFEMESHSVTQAGVQWPDLGSLEVTLLPQPPKVGLQVGGNMPSSFFSIFNRDGVSPCWPGWSLPPDLMIHTPWPPEVLGLQAATVPGLGSLFFLRVLFFKAFIGEIFLRDTKSNSRFLLLVLCSTEKKGINELNFSLNIFLDRWLWRLLQWIWRKLLPGGLVGQLN